(19) United States
(12) Patent Application Publication
(10) Pub. No.: US 2026/0224773 A1
(43) Pub. Date: Aug. 6, 2026

(54) METHOD OF TREATING LOWER BACK PAIN

(71) Applicant: Gelmetix Limited, Nether Alderley (GB)

(72) Inventors: Jane Helen BRAMHILL, Bollington (GB); Anthony John FREEMONT, Sale (GB); Phillippe JENNY, Pessac (FR)

(73) Assignee: Gelmetix Limited, Nether Alderley (GB)

(21) Appl. No.: 19/401,169

(22) Filed: Nov. 25, 2025

Related U.S. Application Data
(63) Continuation of application No. 17/617,525, filed on Jun. 12, 2020, now abandoned.

(30) Foreign Application Priority Data
Jun. 14, 2019 (GB) 1908589.3

(51) Int. Cl. A61L 27/16; A61L 27/52

(52) U.S. Cl. CPC A61L 27/16; A61L 27/52; A61L 2400/06; A61L 2430/38

(57) ABSTRACT

The present invention provides relatively non-invasive treatments generally involving injection of a treatment composition to a subject's target site which exhibits a defined degradation state (e.g. typically “partially-degraded target sites”) and/or symptoms corresponding with such a degradation state. Such target sites, which are suitably specific regions of a human or animal body (e.g. an intervertebral disc or a component part thereof, such a nucleus pulposus), typically degrade to the defined degradation state as a consequence of biological degeneration at the target site, in particular cellular and/or extracellular degradation, and administration thereto of treatment compositions of the invention can facilitate physical and/or biochemical restoration of such target sites.

### CROSS-REFERENCE TO RELATED APPLICATIONS

This present application is a Continuation of U.S. patent application Ser. No. 17/617,525, filed Dec. 8, 2021, which is a National Stage Entry of International Patent Application No. PCT/GB2020/051433, filed Jun. 12, 2020, which claims foreign priority from British Patent Application No. GB 1908589.3, filed Jun. 14, 2019, each of which is incorporated herein by reference in its entirety.

### INTRODUCTION

The present invention relates to methods of diagnosing and methods of treating cellular decline and/or tissue damage, especially cellular decline within and/or damage of stressed tissues, for example, including load-bearing tissues, joints, and tissues present within joints and load-bearing elements of the human and animal body. The present invention also relates to materials, devices, articles, medicaments, and biomarkers for use in such methods.

### BACKGROUND

Reportedly, spinal pain affects up to 80% of the population.¹ A common cause of such pain is Degenerative Disc Disease (DDD) which, though not a “disease” as such, is characterised by the natural degradation of intervertebral discs of the spine, generally as a consequence of natural wear-and-tear and/or minor injuries which over time leads to declining water content within the discs. Such a decline in water content affects the structure and function of the intervertebral discs and can result in acute (and often chronic) pain, typically discogenic pain (i.e. whose pain source is one or more intervertebral discs, typically in the lumbar region of the spine) though other types of pain may develop over time. In some cases, the pain can be very severe and physically debilitating. Chronic lower back pain (CLBP) is estimated to cost the UK and US economies approximately $85.9 billion and £1.6 billion respectively, a figure higher than any other condition including arthritis, cardiovascular disease and cancer.²

### Physical Structure and Functionality of Intervertebral Disks

The spinal column (vertebral column), common to all vertebrates (animal and human), is an elongate segmented array of vertebrae (bones) separated by intervertebral discs lying there between, which houses the spinal canal through which the all-important spinal cord extends. Intervertebral discs act as ligaments by holding together neighbouring vertebrae via fibrocartilaginous joints, which permit a degree of relative movement and spinal flexibility. They also share a load-supporting function with the vertebrae, but most importantly the intervertebral discs serve as natural shock-absorbers.

The load-supporting and shock-absorbing capacity of intervertebral discs primarily stems from the biomechanical and chemical properties of their central components, namely the nucleus pulposus (NP) and its encapsulating annulus fibrosus (AF) and vertebral endplates (VEP). In particular, the swelling pressure of the nucleus pulposus resisted by tension in the annulus fibrosus affords intervertebral discs their biomechanical properties, and degradation of either may manifest itself in a loss in disc height, altered compressive stiffness, delamination, and increased risk of disc prolapse.³

### Biochemistry of Intervertebral Disks

In healthy intervertebral discs, the nucleus pulposus is a well-hydrated gelatinous mass which typically contains between 1-3 wt % cells (including inter alia chondrocyte-like cells, notochordal cells, and NP stem/progenitor cells), with the remainder being an extra-cellular matrix (ECM) and 80-90 wt % water.⁴ This high water content distributes compressive hydraulic pressures and compressive shocks throughout the whole disc to thereby minimise any concentration of stress. The biomechanical properties of intervertebral discs are dependent on the continued biosynthetic activity of IVD cells and their control of degradation in the extracellular matrix.

The ECM contains a range of extracellular molecules, typically secreted by local cells (i.e. those present within the NP), whose functions vary from cellular support/nutrition, growth-factor storage, tissue segregation, and regulation of intercellular communication. An additional function of certain extracellular molecules, in the specific context of intervertebral discs, is to facilitate generation and maintenance of appropriate biomechanical properties to allow NPs to effectively serve their load-supporting and shock-absorbing functions. Such additional functions are well served by those ECM molecules which attract large amounts of water to ensure the NP is well-hydrated.

In contrast to the composition of the AF, which is high in collagen (mainly type I collagen, though higher amounts of type II collagen are present at the interface with the NP) and low in proteoglycans, the NP is low in collagen (mainly type II collagen is present) and high in proteoglycans. In fact, the key ECM molecules of the NP responsible for its immense water-binding capacities are glycosaminoglycan (GAG)-bearing proteoglycans (PGs), which are essentially highly glycosylated proteins with an abundance of negatively-charged sulphate groups borne by the glycosaminoglycan side chains. The sulphate groups in particular provide tremendous osmotic allurement towards water and sodium cations and are responsible for the gelling, swelling, and hydration properties of the PGs. Though there are several types of GAG side chains amongst the many PGs present within the ECM—including inter alia chondroitin sulphate/dermatan sulphates, heparan sulphate/chondroitin sulphates, chondroitin sulphates (including aggrecan), keratan sulphates, and hyaluronan aggrecan is the key component of the ECM for imbibing NPs with their load-supporting and shock-absorbing properties.

Another important class of PGs are small leucine-rich proteoglycans (SLRP), which are crucial for NP cell survival. SLRPs sequester and increase the local bioavailability of growth factors (important for NP vitality, reparation, and NP cell revitalisation) and facilitate signal transduction allowing for effective communication between local cells and the ECM. For the SLRPs to perform their role effectively, the ECM itself must be conducive to the diffusion of molecules, and thus sufficiently hydrated. As aforementioned, sufficient hydration relies on the PGs which are typically expressed by the NP cells. It is thus clear that there is a delicate interplay between the NP cells and their associated ECM—the continued viability of the NP cells is important for an optimal ECM, and the viability of the NP cells depends on the ECM.

### Process of Intervertebral Disk Degeneration

With age, injury, and daily wear-and-tear, the composition of both the NPs and AFs changes in a manner which results in water loss, which in turn can hasten structural damage. For instance, NP degeneration may begin with the depletion of key ECM molecules, including PGs such as aggrecan and SLRP and also type II collagens. Without wishing to be bound by theory, such depletion may arise at least in part from a changing NP cell landscape, for instance a relative decline in notochordal cells and relative increase in chondrocyte-like cells. Such depletion of key ECM molecules may also arise through a variety of biochemical degradation pathways, including protease scission leading to a loss of GAGs and a consequential reduction in net swelling pressure (since the ECM becomes less hydratable).

The reduction in water content within the NPs can compromise molecular diffusion within the NPs. Since cells depend on diffusion, to receive oxygen and nutrients from capillaries in adjacent vertebrae and surrounding vascularized tissues, and remaining SLRPs require sufficient diffusion to perform their revitalising function, cell viability will inevitably decline and ultimately progressive cell death will precipitate deterioration of the ECM itself because the expression of key ECM molecules will decline. This in turn leads to further cell death, and a vicious circle.

Moreover, such water-deficiency leads to declining load-support/shock-absorbent performance which places greater stresses upon the endplates (VEPs) and encircling AF and can cause the disc itself to become misshapen. The extra stresses borne by these rigid outer elements (AF and VEPs) can lead to their weakening, and ultimately fissures, tears, and clefts can develop within the AF and/or VEP (and indeed, within the NP itself), providing further opportunities for water to haemorrhage from the inner NP and new opportunities for the ingrowth of nerves and blood vessels (causing pain and inflammation). Three broad categories of tears include circumferential tears (delaminations); peripheral rim tears; and radial fissures, which can extent to the periphery of the AFs. In certain scenarios, NP material can seep out through tears in the AF, leading to a herniated disc. A vicious circle can ensue, especially as it can be difficult to identify this unfolding degradation before it is too late to remedy.

A vertebrate's body will typically react to intervertebral disc degradation and the consequences thereof by, for instance: generating bone spurs within the collapsing gap between neighbouring vertebrae (which can cause significant pain if such bone spurs grow into the spinal cord and surrounding nerve roots); replacing the original gelatinous NP with fibrocartilage material (with type I collagen replacing the previously-predominant type II collagen); developing granulation tissue; replacing the AF fibres with coarsened and hyalinized fibers; and inflammation. All such reactions can lead to greater pain and further structural problems.

In particular, degradation can be recognised via the inflammatory response thereto, notably the increased production of proinflammatory cytokines such as interleukin 1 (IL-1), especially interleukin 1□ (IL-1□), which stimulate NP cells to secrete neurotropic factors which, alongside a declining GAG content, can encourage neoinnervation and neovascularization at the vertebral endplate and peripheral annulus.⁵ Degradation also causes increased secretion of matrix metalloproteinases (MMPs) and aggrecanases, which destroy the GAG within the NP.⁶

### Traditional Pain and Clinical Evaluation Techniques

Treatment decisions in respect of back pain are usually based on history, physical examination, and imaging results. Identifying the source of the pain can be very important since outcomes of surgical and nonsurgical treatments often depend on the source of the pain. The source of lower back pain may be one of many pain generators including the intervertebral disk, innervated structures within the spinal column (facet joints, endplate), neural elements within the spinal canal (nerve radicles, cauda equina), and extraspinal disease (genitourinary, vascular, gastrointestinal, myofascial sacroiliac). In some cases, no specific cause of pain may be identified. Pain in the centre or slightly lateral to the midline in the lower lumbar region or pain referred to the buttock and posterior thigh with prolonged sitting or flexion suggests discogenic origin (i.e. discogenic pain) whereas radiculopathy or sciatica with positive straight leg raising, dermatomal pain distribution, and neurologic deficits is more suggestive of herniated disks, or bulging disc material or a narrowing of the lateral foramen. Discography (e.g. imaging intervertebral disk following administration of contrast media to the disk) was long considered the reference standard for diagnosing discogenic pain.⁵ However, such reference standards can be somewhat crude and sometimes lead to incorrect clinical evaluations, they are more effective at diagnosing mild degenerative conditions and less effective at diagnosis of earlier stages of degeneration. As such, recent advances in radiographic imaging, as discussed below, may provide invaluable tools permitting better discrimination and classification of degenerative disorders and associated pain sources.

### General Points on Existing Treatments

Degenerative disc disease, and associated symptoms, can sometimes be treated non-surgically, for instance through certain physical therapies (e.g. physiotherapy, chiropractory), the use of anti-inflammatory medicaments, epidural steroidal injection, and traction. However, such treatments may be ineffective, especially during the later stages of DDD, and thus many circumstances arise where surgery becomes necessary for this late stage disease. Such surgical therapies are often invasive and dangerous, and may include one or more of: anterior cervical discectomy and fusion; cervical corpectomy; dynamic Stabilisation; facetectomy; foraminotomy; intervertebral disc annuloplasty; intervertebral disc arthroplasty; laminoplasty; laminotomy; microdiscectomy; percutaneous disc decompression; spinal decompression; and/or spinal laminectomy.

Non-surgical “light-touch” treatments are generally preferred during early stages of disc degeneration, during which discogenic pain (as distinct from nerve-derived pain) is often the most prevalent symptom, because major treatments such as surgery can carry significant costs and risks and are thus generally postponed until deemed absolutely necessary. As such, patients must endure several years of horrendous symptoms (e.g. discogenic pain) whilst contemplating the unpleasant prospect of the risky surgery to come.

### Existing Treatments for Replacing Nucleus Pulposus

Nucleus pulposus replacement has been the focus of much research since the 1950s, which heralded the limited replacement of the NP following injection of methylacrylic into the IVD space after an open discectomy. Since then, various Nucleus Pulposus Replacement (NPR) device technologies have been developed.

In early developments of NPRs, it was recognised that NP prostheses must comfortably fill the disc space to prevent excessive movement of the implant, that could later precipitate implant extrusion. The implant should be designed so that it may be inserted using a minimally invasive or minimal access approach that limits destruction of surrounding tissue, enhancing stability of implanted components and favouring expulsion.¹¹

Currently, the most common injectable elastomers, with approximate biomechanical properties of the nucleus pulposus, used within the intervertebral disk space are silicone and polyurethane. These materials can be implanted through a minimally invasive approach by injecting the implant through a small annulotomy. In principle, this decreases the risk of implant extrusion. In situ curable implants conform to the nucleotomized cavity, which maximizes filling of available space. Complete filling also improves implant stability.

To date the most widely studied nucleus replacement device has been the Prosthetic Disc Nucleus (Raymedica, Inc., Bloomington, MN) which has been implanted in over 550 patients.¹² This PDN is essentially a hydrogel pellet, formed from a copolymer of polyacrylonitrile (non-hydrophilic) and polyacrylamide (hydrophilic), encased in a polyethylene jacket. The encased PDN pellets absorb 80% of their own weight in water, causing them to swell and thereby restore or maintain disc height. The swelling needs to be restrained to avoid endplate fracturing, so the surrounding polyethylene jacket is very strong (i.e. high molecular weight and linear polyethylene fibers). This jacket also minimizes horizontal spreading, thereby maintaining the shape of the implant.

In other developments, a DASCOR™ device deploys a methylenediphenyldiisocyanate-based polyurethane two-part reactive system which is injected under controlled pressure, whilst still in a liquid state, through a catheter to an expandable balloon that is placed in a prepared nucleotomy space. The resulting polymer cures within minutes within the balloon. Once cured in situ the material does not rely on additional hydration. The device can be surgical implanted via a minimally invasive posterolateral approach, with the potential for a minimally invasive endoscopic approach under sedation only. Fluoroscopy is used for monitoring during the surgery. Though these devices are effective, unfortunately some patients have reportedly suffered from explantation of the device and resumed back pain following migration due to the size of the implant.

Another product in this area is the Aquarelle™ pre-formed nucleus pulposus replacement (Stryker Spine, Allendale, NJ). The Aquarelle™ product is a hydrogel implant composed primarily of polyvinyl alcohol, which can be implanted through a small annulotomy by using a 4- to 5-mm tapered cannula. The insertion of the component can be achieved through a lateral or posterior technique. Though this device has shown some promise, extrusion of the material has been reported.

An in-situ curable injectable hydrogel known as NuCore® Injectable Nucleus (Spine Wave, Inc.) has been studied as a possible nucleus replacement following microdiscectomy. The material, which is a protein which mimics the nucleus pulposus, is injected through an annular defect so as to fill the nuclear void and adhere to the surrounding discal tissue as it cures. The material is designed to replace nucleus tissue lost to herniation and discectomy, and subsequently prevent or retard further degeneration of the disc.

NeuDisc™ is another hydrogel implant, again designed to mimic the natural nucleus pulposus, which exists in a layered structure with Dacron mesh ranging from 6.5 mm to 15 mm in size. In one approach the implant was introduced into the intervertebral disc using ALPA (anterolateral transpsoatic approach), and in another approach a posterolateral endoscopic approach was used. It was found that successful insertion of the implants was dependent on complete nucleotomy and appropriate implant positioning and size.

BioDisc™ Spinal Disc Repair is a technology which leverages in situ polymerising of a protein hydrogel as an adjunct to discectomy, with the intention to reduce motion segment instability, reduce recurrent nuclear herniation, and preserve disc height. These implants are introduced to a subject via annulotomy injection of precursor materials into a cavity created by standard open discectomy, and the implant polymerises within 2 minutes. Positional MRI scans on subjects treated with BioDisc™ show that the implant is securely seated within the annulus without migration or expulsion.

All of the aforementioned techniques involve an unwelcome degree of invasiveness, surgical preparation, use of secondary articles (e.g. jackets, balloons, catheters), or implants that are vulnerable to migration and/or expulsion.

### Diagnostic Techniques for New Generation of Potential Treatments

Various alternative treatments and imaging strategies have been proposed and developed for use in tackling DDD.⁵ Such alternative treatments generally focus on genetics, nutrition, cell senescence, apoptosis, and imbalances between anabolic and catabolic processes within the IVD. Developments underway include the intradiscal injection of growth factors, inflammatory inhibitors, proteinases inhibitors, intracellular regulatory compounds, genes, and cells. All such treatments aim to replenish disc cells and their surrounding ECM and thus these treatments are more applicable in the early stages of disc degradation (i.e. before degradation has progressed beyond the point of no return). As such, of key importance is the ability to diagnose early stage disc degradation and, ideally monitor treatment outcomes.

Magnetic resonance imaging (MRI) is often used to assess spinal damage, but T2-weighted MR images are particularly useful in grading (i.e. classifying) disk degeneration.⁷ The Pfirrmann system utilises signal intensities and morphology to rank disk degeneration according to fives grades. The five grades cover normal-appearing child and adult disks (grades I and II) to disks with diminished signal intensity (grade III) and disks with progressively greater loss of height and of other normal features (grades IV, V). Grade III disks demonstrate biochemical and biomechanical changes, including diminished proteoglycan content and stiffness as compared with grade I and II disks. Grade III disks also exhibit radial fissures of the annulus fibrosus, which may or may not be detected as a linear region of high intensity.⁵ Depending on the nature, location, and/or extent of such radial fissures, nucleus pulposus may well be able to leak out through the fissures. Unsurprisingly, therefore, disk degeneration grading schemes are considered to be particularly valuable for determining appropriate points for intervention at the early degeneration stages. Standard MRI tends to be performed using 1.5T MRI systems, but other systems, such as the 3T MRI systems being used for spinal research, may also be used and the Pfirrmann scale remains applicable in both cases. The “T” of “1.5T” and “3T” means Tesla, and thus refers to the strength of the magnet used within any particular MRI machine.

Newer imaging techniques, such as T2 mapping, diffusion imaging, T1p mapping, MR spectroscopy, and nuclear imaging have also shown great promise in providing a means for determining appropriate points during the early degeneration stages for revitalising clinical interventions.

T2 relaxation time is represented as a decay constant for MR T2 signal intensity in MRI. Such T2 relaxation times can be mapped to intrinsic properties of tissues which, in the case of intervertebral discs, reflects a disk's molecular environment in terms of water, proteins, collagen, and other pertinent solutes. A dedicated fast spinecho T2-weighted multiecho sequence provides a T2 measurement in about 6 minutes with a 1.5-T system. Intervertebral disk T2 relaxation times correlate with hydration and to a lesser extent with proteoglycan content and (negatively) with collagen, thus providing a useful indication of the state of a disk.⁵ T2 relaxation provides a means of continuous measurement of disk aging or degeneration, since even small changes in the disk may be measured over time. Through T2 relaxation measurements, diurnal variations in water content may be ascertained as well as the effect of normal aging and also the effect of degeneration on intervertebral disks. In general, T2 relaxation times measured in respect of the nucleus pulposus should change by approximately 10% per decade based on normal disk aging, and change 20%-50% with development of Pfirrmann grade III degenerative changes. T2 measurements may be useful for monitoring biochemical changes in intervertebral disks over a continuous period both before and even after intradiskal therapies.⁵

The T1r time constant obtained by means of spin-lock MR imaging A technique is very sensitive to the GAG content in cartilage, since it is related to slow motional interactions between macromolecules and bulk water. Though qualitatively similar to T2 mapping, T1r values express a greater dynamic range and thus greater sensitivity to small tissue hydration and proteoglycan content. T1r measurements have thus been usefully deployed to assess the condition of intervertebral disks.⁵

Diffusion Imaging is particularly useful for measuring diffusibility of solutes within an intervertebral disk, and in particular within the nucleus pulposus thereof. Diffusion is key for effective metabolism of the avascular intervertebral disk, and quantitative measurements of diffusion may be performed using T1 signal intensity before and serially after intravenous contrast medium injection into a disk. Diffusibility may be calculated from changes in signal intensity over time after intravenous administration of contrast medium. Such solute diffusion measurements can be illustrative of disk maturation, mechanical loading, and vasodilators on disk diffusion.⁵

Painful disks may be characterized by hypoxia, inflammation, neovascularization, neoinnervation, and decreased GAG levels. Since lactate, alanine, and lipids can accumulate under these pathologic conditions, they may serve as useful biomarkers detectable by MR spectroscopy. MR spectroscopy analysis has shown great promise in distinguishing painful disks from controls on the basis of differing spectra. Of particular use is that spectral analyses have demonstrated significantly lower proteoglycan, GAG/collagen, and GAG/lactate ratios, and a higher lactate/collagen ratio in specimens obtained from disks adjudged to be causing discogenic pain. Signal-to-noise ratios may be dramatically improved with judicious postprocessing such as optimal channel selection, phase rotation error correction, frame editing, frequency shift error correction, and apodization. Using such signal enhancements, in vivo single-voxel MR spectroscopy has allowed differentiation of diskography-confirmed painful disks from asymptomatic controls based on changes in the ratio between proteoglycan and combined lactate/lipid/alanine peaks.

Positron Emission Tomography (PET) may be used to assess lower back pain through the identification of disk inflammation. For example, fluorine 18-labelled fluoro-deoxyglucose (FDG) can diffuse into disks, and uptake can be detected in disks of patients exhibiting lower back pain. Without wishing to be bound by theory, it is thought that increased disk uptake may signify inflammatory processes and reflect senescent changes in disk cells. Given inflammation is a central feature of painful disks, PET may represent another useful technique for selecting patients exhibiting early-stage disk degeneration.⁵

### New Generation of Potential Treatments

Some recently developed early-stage treatments of DDD include:
the intradiscal injection of proteins to upregulate GAG synthesis and promote cell proliferation (e.g. various growth factors);⁵
the intradiscal injection of IL-1 receptor antagonists to inhibit or reverse disk degeneration, since it is known that IL-1 is a proinflammatory cytokine which depletes the ECM of a disk, reduces aggrecan synthesis, and stimulates cartilage degradation;^{5, 8, 9, 10}
gene therapies, for instance, involving the introduction of certain viral vectors encoding therapeutic proteins which correct homeostasis imbalance or retard the loss of GAGs;⁵
various stem cell therapies.⁵

### Object(S) of the Invention

An object of the present invention is to provide an alternative treatment and/or diagnostic strategy to those of the prior art.

Another object of the present invention is to solve at least one problem inherent in the prior art.

Another object of the present invention is to provide an improved treatment and/or diagnostic strategy to those of the prior art. For example, it is desirable to provide a relatively non-invasive therapy which rejuvenates degenerated or partially-degenerated intervertebral discs, or the nucleus pulposus thereof, in a patient population which can benefit most from such therapy.

Another object of the invention is to revitalise cells and/or extra cellular matrices, suitably by altering or (at least partially) restoring a favourable local environmental for the cells and/or extra cellular matrices.

### SUMMARY OF THE INVENTION

The present invention provides effective solutions to problems inherent in the prior art. Such solutions involve relatively non-invasive treatments, especially when compared to traditional surgical treatments. Such treatments generally involve introduction or injection of a treatment composition to a target site of a subject in need of such a treatment. Subjects (whether animal or human) who may benefit most from treatments of the invention (e.g. subjects in need of such treatments) generally exhibit a defined degradation state in respect of one or more target sites (i.e. “partially-degraded target sites”) and/or symptoms corresponding with such a degradation state. Such target sites, which are suitably specific regions of a human or animal body (e.g. an intervertebral disc or a component part thereof, such a nucleus pulposus), typically degrade to the defined degradation state as a consequence of biological degeneration at the target site, in particular cellular and/or extracellular degradation. Though, depending on the definition of the degradation state (i.e. window of degradation in question), such biological degeneration need not necessarily be accompanied by (substantial) structural degradation, in some cases a degree of structural degradation may also be exhibited, though most suitably such structural degradation will not have proceeded beyond a defined point.

Treatments of the invention generally seek to rejuvenate these one or more partially-degraded target sites rather than replacing them. Such rejuvenation suitably involves introduction (typically via injection) to the target site(s) of materials which restore or mimic a degree of healthiness, which suitably in turn prompts an enthusiastic biochemical response that revitalises or further revitalises the target site(s).

In view of the foregoing, it may first be useful to identify subjects who may benefit most from treatments of the invention (i.e. a specific patient population or sub-population), or subjects in need of such treatments. To this end, candidate subjects may be identified by reference to a degradation state of one or more target sites (e.g. partially-degraded target sites) thereof. Subjects identified as exhibiting a defined “degradation state” in respect of one or more target sites, i.e. patients exhibiting one or more partially-degraded target sites, may be considered “a subject in need of” a treatment according to the invention.

According to a first aspect of the present invention there is provided a method of determining a degradation state of a target site, the method comprising:
i) providing degradability data in respect of the target site;
ii) on the basis of the degradability data, determining the degradation state of the target site;
wherein suitably the degradation state is a qualitative and/or quantitative value indicative of a degree of degradation of the target site, and suitably the value facilitates an assessment of likely success of a treatment of the invention upon the target site.

According to a further aspect of the present invention there is provided a method of determining a degradation state of an intervertebral disc or one or more components thereof (suitably within a subject), the method comprising: performing the method of determining a degradation state of a target site; wherein the target site is an intervertebral disc or one or more components thereof (e.g. nucleus pulposus, annulus fibrosus, and/or vertebral endplate(s)). Suitably the degradability data comprises one or more images (e.g. MRI images) of the intervertebral disc or one or more components thereof.

In the context of the present invention, where a degradation state of a given target site is or complies with a pre-defined degradation state, that target site may be termed a “partially-degraded target site”.

According to a further aspect of the present invention there is provided a method of identifying a candidate subject, suitably one in need of (or who may benefit from) a treatment according to the invention, the method comprising:
(i) performing in respect of a subject the method of determining a degradation state of a target site upon one or more target sites of the subject (e.g. to determine whether any of the one or more target sites exhibit a pre-defined degradation state);
(ii) on the basis of degradation state(s) of the one or more target site(s), suitably in comparison to a pre-defined candidate degradation state, determining whether or not the subject is a candidate subject;
(iii) optionally identifying one or more partially-degraded target site(s) by reference to which of the one or more target site(s) comply with a pre-defined degradation state;

wherein the degradation state is a qualitative and/or quantitative value indicative of a degree of degradation of the one or more target regions of the subject, and suitably the value facilitates an assessment of likely success of a treatment of the invention upon the subject or upon the one or more target site(s) of the subject.

According to a further aspect of the present invention there is provided a method of identifying a candidate subject, the method comprising:
(i) performing in respect of a subject the method of determining a degradation state of a target site upon one or more target sites of the subject;
(ii) identifying the subject as a candidate subject if any degradation state(s) of the one or more target site(s) comply with a pre-defined degradation state criterion;
(iii) optionally identifying one or more partially-degraded target site(s) by reference to which of the one or more target site(s) comply with the pre-defined degradation state criterion.

According to a further aspect of the present invention there is provided a method of treating a candidate subject (suitably as defined herein) exhibiting one or more partially-degraded target site(s), the method comprising introducing or injecting a treatment composition into one or more partially-degraded target site(s) of the candidate subject.

According to a further aspect of the present invention there is provided a treatment composition for use in treating a candidate subject (suitably as defined herein) exhibiting one or more partially-degraded target site(s). In this context, “treating” suitably comprises a method of treating a candidate subject as defined herein.

According to a further aspect of the present invention there is provided a method of treating a candidate subject (suitably as defined herein) exhibiting one or more partially-degraded target site(s), the method comprising introducing a hydrogel composition (or post-treatment composition) into one or more partially-degraded target site(s).

According to a further aspect of the present invention there is provided a hydrogel composition (or post-treatment composition) for use in treating a candidate subject (suitably as defined herein) exhibiting one or more partially-degraded target site(s). In this context, “treating” suitably comprises a method of treating a candidate subject as defined herein.

In some embodiments, the treatment composition is (substantially) the same as the hydrogel composition (or post-treatment composition), whilst in some other embodiments the treatment composition transforms or is transformed into the hydrogel composition (or post-treatment composition). As such, a treatment composition may be introduced or injected into one or more partially degraded target site(s) (e.g. as part of a method of administering the treatment composition to a candidate subject) and the treatment composition may transform (suitably via a physical and/or chemical reaction, optionally initiated by an initiator) into the hydrogel composition (or post-treatment composition) (e.g. a part of a method of administering the hydrogel composition or post-treatment composition to a candidate subject). The hydrogel composition (or post-treatment composition) suitably physically and/or chemically mimics healthy non-degenerated target sites.

According to a further aspect of the present invention there is provided a method of revitalising one or more partially-degraded target site(s), the method comprising introducing a treatment composition or a hydrogel composition (or post-treatment composition) into one or more of the partially-degraded target site(s).

According to a further aspect of the present invention there is provided a treatment composition or a hydrogel composition (or post-treatment composition) for use in a method of revitalising one or more partially-degraded target site(s) (suitably as defined herein).

According to a further aspect of the present invention there is provided a method of revitalising cells or cellular function at one or more partially-degraded target site(s), the method comprising introducing a treatment composition or a hydrogel composition (or post-treatment composition) into one or more of the partially-degraded target site(s).

According to a further aspect of the present invention there is provided a treatment composition or a hydrogel composition (or post-treatment composition) for use in a method of revitalising cells or cellular function (suitably as defined herein).

According to a further aspect of the present invention there is provided a method of revitalising an extracellular matrix (ECM) (and optionally also cells associated therewith) at one or more partially-degraded target site(s), the method comprising introducing a treatment composition or a hydrogel composition (or post-treatment composition) into one or more of the partially-degraded target site(s).

According to a further aspect of the present invention there is provided a treatment composition or a hydrogel composition (or post-treatment composition) for use in a method of revitalising an extracellular matrix (ECM) (and optionally also cells associated therewith) (suitably as defined herein).

According to a further aspect of the present invention there is provided a method of retarding degradation (particularly the rate of degradation) of a partially-degraded target site, the method comprising introducing a treatment composition or a hydrogel composition (or post-treatment composition) into one or more of the partially-degraded target site(s). Such degradation may, for example, refer to any one or more of physical/structural degradation, biochemical degradation, cellular degradation, degradation of an extracellular matrix. The degradation may extend beyond the target site, for example, to neighbouring sites or neighbouring tissues. The degradation may be or relate to degenerative disk disease (DDD). In an embodiment, retarding degradation comprises inhibiting progress of degenerative disc disease and/or cartilage degradation.

According to a further aspect of the present invention there is provided a treatment composition or a hydrogel composition for use in a method of retarding degradation of a partially-degraded target site (suitably as defined herein).

According to a further aspect of the present invention there is provided a method of treating or alleviating pain at or derived from one or more partially-degraded target site(s), the method comprising introducing a treatment composition or a hydrogel composition into one or more of the partially-degraded target site(s). The pain may, for instance, be discogenic pain and the target site(s) may be intervertebral discs (or the nucleus pulposus thereof).

According to a further aspect of the present invention there is provided a treatment composition or a hydrogel composition for use in a method of treating or alleviating pain at one or more partially-degraded target site(s) (suitably as defined herein).

According to a further aspect of the present invention there is provided a method of hydrating or rehydrating (or increasing water content of) an extracellular matrix (ECM) at one or more partially-degraded target site(s) (suitably of a candidate subject), the method comprising introducing (suitably via injection) a treatment composition or hydrogel composition to, into, around, and/or in the proximity of the extracellular matrix.

According to a further aspect of the present invention there is provided a treatment composition or a hydrogel composition for use in a method of hydrating or rehydrating (or increasing water content of) an extracellular matrix (ECM) at one or more partially-degraded target site(s) (suitably as defined herein).

According to a further aspect of the present invention there is provided a method of inhibiting the production, secretion, or accumulation of one or more inflammatory cytokines (e.g. IL-1, particularly IL-1□) at one or more partially-degraded target site(s), the method comprising introducing a treatment composition or a hydrogel composition into one or more of the partially-degraded target site(s).

According to a further aspect of the present invention there is provided a treatment composition or a hydrogel composition for use in a method of inhibiting the production, secretion, or accumulation of one or more inflammatory cytokines (e.g. IL-1, particularly IL-1□) at one or more partially-degraded target site(s) (suitably as defined herein).

Suitably, in the above methods and compositions for use, the one or more partially-degraded target site(s) are part of or within a candidate subject, and suitably the method is performed in vivo. However, the method may be performed in vitro.

According to a further aspect of the present invention there is provided a method of treating a candidate subject (suitably as defined herein) exhibiting one or more partially-degraded target site(s), the method comprising:
(i) identifying a candidate subject (suitably via a method as defined herein)
(ii) identifying one or more partially-degraded target site(s) of the candidate subject;
(iii) introducing or injecting a treatment composition or a hydrogel composition into one or more partially-degraded target site(s) of the candidate subject;
(iv) optionally thereafter examining the one or more target site(s) into which the treatment composition or hydrogel composition was introduced (e.g. via a method of determining a degradation state of a target site as defined herein) to assess treatment outcomes.

According to a further aspect of the present invention there is provided a method of introducing a hydrogel composition into a partially-degraded target site (suitably of a candidate subject), the method comprising injecting a treatment composition (or an injectable form of the hydrogel composition) into the partially-degraded target site; and thereafter transforming the treatment composition (or an injectable form of the hydrogel composition) or allowing the same to transform into the hydrogel composition (or a non-injectable form thereof) within the target site (suitably of a candidate subject).

In further aspects of the present invention there is provided a treatment composition or hydrogel composition for use in the manufacture of an active medical device for performing any of the aforementioned methods which involve either or both the treatment composition and/or hydrogel composition.

According to a further aspect of the present invention there is provided a post-treatment composition.

According to a further aspect of the present invention there is provided a treatment composition. The treatment composition may be formed by mixing together an activatable composition and an activator composition.

According to a further aspect of the present invention there is provided a kit comprising an activatable composition and an activator composition.

According to a further aspect of the present invention there is provided an activatable composition.

According to a further aspect of the present invention there is provided an activator composition.

Any features, including optional, suitable, and preferred features, described in relation to any particular aspect of the invention may also be features, including optional, suitable and preferred features, of any other aspect of the present invention.

### BRIEF DESCRIPTION OF THE DRAWINGS

For a better understanding of the invention, and to show how embodiments of the same are put into effect, reference is now made, by way of example, to the following diagrammatic drawings, in which:

FIG. 1 shows a sheep, and labels the specific vertebrae that were investigated/treated.

FIG. 2 shows a photograph of an excised sheep lumbar spine.

FIGS. 3A-C show microscopic images of an IVD displaying evidence of degeneration by way of: A) cell clusters; B) slits; and C) end-plate lesions.

FIG. 4 shows microscopic images of histological disc tissue (left) incorporating viable cells (tiny dark nuclei) and fragmented post-treatment composition (i.e. after curing in vivo) on the right.

FIG. 5 shows microscopic images of viable cells (dark round nuclei) next to spaces left by the post-treatment composition.

FIG. 6 shows a microscopic image of IVD disc tissue with DXM gel filling two separate tears within the disc.

FIG. 7 shows an microscopic image of IVD disc tissue injected with PBS, leaving mangled tissue in the centre of the disc.

FIG. 8 shows an X-ray image, captured via C-arm, showing the relevant vertebrae, with dark regions on the right hand two discs demonstrating the treatment gel within the disc.

FIGS. 9A-B shows X-ray images: A) at the time of injection; and B) at the time of sacrifice.

### DETAILED DESCRIPTION OF THE INVENTION

### Definitions

Unless otherwise stated, the following terms used in the specification and claims have the following meanings set out below.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

For the avoidance of doubt, it is hereby stated that the information disclosed earlier in this specification under the heading “Background” is relevant to the invention and is to be read as part of the disclosure of the invention.

Unless stated otherwise, any reference herein to an “average” value is intended to relate to the mean value.

Where a composition is said to comprise a plurality of stipulated ingredients (optionally in stipulated amounts of concentrations), said composition may optionally include additional ingredients other than those stipulated. However, in certain embodiments, a composition said to comprise a plurality of stipulated ingredients may in fact consist essentially of or consist of all the stipulated ingredients.

Herein, where a composition is said to “consists essentially of” a particular component, said composition suitably comprises at least 70 wt % of said component, suitably at least 90 wt % thereof, suitably at least 95 wt % thereof, most suitably at least 99 wt % thereof. Suitably, a composition said to “consist essentially of” a particular component consists of said component save for one or more trace impurities.

Where the quantity or concentration of a particular component of a given composition is specified as a weight percentage (wt % or % w/w), said weight percentage refers to the percentage of said component by weight relative to the total weight of the composition as a whole. It will be understood by those skilled in the art that the sum of weight percentages of all components of a composition will total 100 wt %. However, where not all components are listed (e.g. where compositions are said to “comprise” one or more particular components), the weight percentage balance may optionally be made up to 100 wt % by unspecified ingredients (e.g. a diluent, such as water, or other non-essentially but suitable additives). Most suitably, the sum of wt % of stipulated ingredients does not exceed 100 wt % and any combinations of wt % that would do so would by definition be excluded.

Herein, unless stated otherwise, the term “parts” (e.g. parts by weight, pbw) when used in relation to multiple ingredients/components, refers to relative ratios between said multiple ingredients/components. Expressing molar or weight ratios of two, three or more components gives rise to the same effect (e.g. a molar ratio of x, y, and z is x1:y1:z1 respectively, or a range x1-x2:y1-y2:z1-z2). Though in many embodiments the amounts of individual components within a composition may be given as a “wt %” value, in alternative embodiments any or all such wt % values may be converted to parts by weight (or relative ratios) to define a multi-component composition. This is so because the relative ratios between components is often more important than the absolute concentrations thereof in the compositions of the invention. Where a composition comprising multiple ingredients is described in terms of parts by weight alone (i.e. to indicate only relative ratios of ingredients), it is not necessary to stipulate the absolute amounts or concentrations of said ingredients (whether in toto or individually) because the advantages of the invention can stem from the relative ratios of the respective ingredients rather than their absolute quantities or concentrations. However, in certain embodiments, such compositions consists essentially of or consist of the stipulated ingredients and a diluents (e.g. water).

The term “mole percent” (i.e. mol %) is well understood by those skilled in the art, and the mol % of a particular constituent means the amount of the particular constituent (expressed in moles) divided by the total amount of all constituents (including the particular constituent) converted into a percentage (i.e. by multiplying by 100). The concept of mol % is directly related to mole fraction.

The term “substantially free”, when used in relation to a given component of a composition (e.g. “a composition substantially free of compound X”), refers to a composition to which essentially none of said component has been added. When a composition is “substantially free” of a given component, said composition suitably comprises no more than 0.001 wt % of said component, suitably no more than 0.0001 wt % of said component, suitably no more than 0.00001 wt %, suitably no more than 0.000001 wt %, suitably no more than 0.0000001 wt % thereof, most suitably no more than 0.0001 parts per billion (by weight).

The term “entirely free”, when used in relation to a given component of a composition (e.g. “a composition entirely free of compound X”), refers to a composition containing none of said component.

Herein, in the context of the present specification, a “strong acid” is suitably one having a pKₐ of −1.0 or less, whereas a “weak acid” is suitably one having a pKₐ of 2.0 or more. Herein, in the context of the present specification, a “strong base” is suitably one whose conjugate acid has a pKₐ of 12 or higher (suitably 14 or higher), whereas a “weak base” is suitably one whose conjugate acid has a pKₐ of 10 or less.

Unless stated otherwise, references herein to a “pKa” should be construed as a pKa value in water at standard ambient temperature and pressure (SATP), suitably of the conjugate acid of the relevant species.

Suitably, unless stated otherwise, where reference is made to a parameter (e.g. pH, pKa, etc.) or state of a material (e.g. liquid, gas, etc.) which may depend on pressure and/or temperature, suitably in the absence of further clarification such a reference refers to said parameter at standard ambient temperature and pressure (SATP). SATP is a temperature of 298.15 K (25° C., 77° F.) and an absolute pressure of 100 kPa (14.504 psi, 0.987 atm).

The term “treatment”, and the therapies encompassed by this invention, include the following and combinations thereof: (1) inhibiting, e.g. delaying initiation and/or progression of, an event, state, disorder or condition, for example arresting, reducing or delaying the development of the event, state, disorder or condition, or a relapse thereof in case of maintenance treatment or secondary prophylaxis, or of at least one clinical or subclinical symptom thereof; (2) preventing or delaying the appearance of clinical symptoms of an event, state, disorder or condition developing in an animal (e.g. human) that may be afflicted with or predisposed to the state, disorder or condition but does not yet experience or display clinical or subclinical symptoms of the state, disorder or condition; and/or (3) relieving and/or curing an event, state, disorder or condition (e.g., causing regression of the event, state, disorder or condition or at least one of its clinical or subclinical symptoms, curing a patient or putting a patient into remission). The benefit to a patient to be treated may be either statistically significant or at least perceptible to the patient or to the physician. It will be understood that a medicament will not necessarily produce a clinical effect in each patient to whom it is administered; thus, in any individual patient or even in a particular patient population, a treatment may fail or be successful only in part, and the meanings of the terms “treatment”, “prophylaxis” and “inhibitor” and of cognate terms are to be understood accordingly. The compositions and methods described herein are of use for therapy and/or prophylaxis of the mentioned conditions.

The term “prophylaxis” includes reference to treatment therapies for the purpose of preserving health or inhibiting or delaying the initiation and/or progression of an event, state, disorder or condition, for example for the purpose of reducing the chance of an event, state, disorder or condition occurring. The outcome of the prophylaxis may be, for example, preservation of health or delaying the initiation and/or progression of an event, state, disorder or condition. It will be recalled that, in any individual patient or even in a particular patient population, a treatment may fail, and this paragraph is to be understood accordingly.

The term “inhibit” includes reference to delaying, stopping, reducing the incidence of, reducing the risk of and/or reducing the severity of an event, state, disorder or condition. Inhibiting an event, state, disorder or condition may therefore include delaying or stopping initiation and/or progression of such, and reducing the risk of such occurring.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

Herein, unless stated otherwise, all chemical nomenclature may be defined in accordance with IUPAC definitions.

### Ketones, Aldehydes, Sugars, Etc.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “(m-nC)” or “(m-nC) group” used alone or as a prefix, refers to any group having m to n carbon atoms.

An “alkylene,” “alkenylene,” or “alkynylene” group is an alkyl, alkenyl, or alkynyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

The term “halo” refers to fluoro, chloro, bromo and iodo.

Wherever groups with large carbon chains are disclosed (e.g. (1-12C)alkyl, (1-8C)alkenyl, etc.), such groups may optionally be shortened, for instance containing a between 1 and 5 carbons (e.g. (1-5C)alkyl or (1-5C)alkenyl), or contain between 1 and 3 carbons (e.g. (1-3C)alkyl or (1-3C)alkenyl instead of (1-12C)alkyl or (1-8C)alkenyl).

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

Herein, the term “particle size” or “pore size” refers respectively to the length of the longest dimension of a given particle or pore. Particle and pore sizes may be measured using methods well known in the art, including a laser particle size analyser and/or electron microscopes (e.g. transmission electron microscope, TEM, or scanning electron microscope, SEM).

In the description of the synthetic methods described below and in the referenced synthetic methods that are used to prepare the staring materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated which are within the ordinary skill of an organic chemist.

“Discogenic pain” is a term of art referring to pain originating from intervertebral discs, usually as a result of DDD, and typically arises from stimulation of pain sensitive afferents within the annulus fibrosus. This is distinct from sciatica or pain associated with disc herniation or radiculopathy.

### General Points Regarding the Invention

The present invention takes advantage of the very synergy between cells and their extracellular matrix (ECM) which increases their vulnerability to degradation. A quantity of a gel, potentially a non-load-bearing quantity thereof, can be introduced into a damaged portion of a gelatinous ECM and, by physically mimicking the extracellular matrix, can rejuvenate cells served by the ECM.

The essence of the invention is to successfully identify the appropriate patient population (i.e. candidate subject) and relevant target site(s) therein that can benefit most from administration of a minimal quantity of an appropriate treatment composition using minimally invasive techniques. In so doing, the present invention can save a patient from unnecessary suffering, channel a patient's own intrinsic biochemistry to rescue target site(s) before damage has proceeded beyond the point of no return, alleviate future needs for potentially-dangerous invasive techniques, and offer healthcare providers a cheaper and less risky alternative to existing treatments.

### Target Sites for Treatment

The present invention generally involves examination of target sites in order to establish whether or not any target sites exhibit a particular degree of degradation. Those targets sites identified as exhibiting a pre-defined degree of degradation are often referred to herein a “partially-degraded target sites”. Having identified partially-degraded target sites, decisions may be taken regarding whether or not such target sites would benefit from treatments according to the invention. Suitably target sites are a part of a subject (e.g. human or animal subject), and partially-degraded target sites are part of a candidate subject (e.g. a human or animal subject who may benefit from or be in need of treatments of the invention).

In the context of the invention, suitably the target site is or comprises an extracellular matrix (ECM) (a “target ECM”), and suitably is or comprises a hydrated target ECM. References herein to a “target site” may, where context-appropriate, be a reference to a target ECM. The target ECM suitably comprises a range of extracellular compounds. Suitably, some, many or all of the extracellular compounds are secreted by peripheral (i.e. surrounding) and/or internal (mixed within the ECM) cells associated with the target ECM (“ECM cells”). Together the extracellular compounds suitably provide structural and/or biochemical support to these ECM cells.

ECMs are commonplace throughout human and animal bodies (in all tissues and organs), and serve many functions (some of which are described in the background section of the present specification) including inter alia providing support, segregating tissues from one another, regulating intercellular communication, and generally regulating dynamic behaviour of associated ECM cells. The dynamic behaviour of ECM cells is not only influenced by the biochemical nature of the ECM but also its physical nature. The stiffness and elasticity of ECMs can, in particular, impact on cell migration as well as gene expression, differentiation, and apoptosis, since ECM cells are generally actively and dynamically sensitive to ECM rigidity and tend to migrate preferentially towards stiffer surfaces. ECM cells may also adjust gene expression accordingly to the prevailing elasticity.

The prevailing stiffness/elasticity properties of a given ECM are primarily influenced by the relative collagen vs elastin concentrations, but also relative concentrations of type I vs type II collagen. As a consequence, treatments of the invention can be remarkably effective primarily due to their impact on the physical properties of a target ECM.

The extracellular compounds of the target ECM suitably comprise one or more collagens, suitably selected from fibrillar collagens, and most suitably comprises type II collagen. The extracellular compounds of the target ECM suitably comprise one or more glycosaminoglycans (GAGs), suitably proteoglycans (PGs), and most suitably comprises aggrecan. The extracellular compounds of the target ECM suitably comprise a combination of collagen(s) and glycosaminoglycan(s), most suitably a combination of type II collagen and aggrecan. The extracellular compounds suitably comprise type II collagen at a higher concentration than type I collagen. The extracellular compounds of the target ECM may comprise or be substantially free of elastin. The extra cellular compounds are suitably (substantially) free from IL-1, especially IL-1□, cytokines.

Suitably the target ECM comprises the extracellular compound and water. Suitably 10-98 wt % of the target ECM is water, more suitably 50-95 wt %, more suitably 70-92 wt %, most suitably 80-90 wt % water. The target ECM is suitably a hydrogel.

In a particular embodiment, the target ECM comprises or contains ECM cells mixed therewith (e.g. which are suitably suspended in the ECM or diffusing therethrough), suitably 0.1-10 wt % ECM cells by weight of the total mixture of target ECM and ECM cells, more suitably 0.5-5 wt %, most suitably 1-3 wt % ECM cells. Though ECM cells may not per se be considered a constituent part of the target ECM, as ECM cells may be mixed into/with the target ECM (e.g. as per a nucleus pulposus), references herein to an ECM may include a mixture of ECM and ECM cells (i.e. the term ECM is not intended to exclude circumstances in which ECM cells are mixed thereinto). As such, a nucleus pulposus may be considered a target ECM in that a nucleus pulposus comprises a mixture of both an ECM hydrogel and nucleus pulposus cells.

The ECM cells may be selected from the group consisting of chondrocyte cells, chondrocyte-like cells, notochordal cells, fibroblasts, stem cells, or any combination thereof. Suitably the ECM cells comprise chondrocyte-like cells. Suitably the ECM cells comprise chondrocyte-like cells and notochordal cells.

The target site may suitably be a cartilaginous target site, suitably comprising an ECM or target ECM, suitably as defined herein. As such, the cartilaginous target site suitably is or comprises cartilage. Such a cartilaginous target site may be located within a subject (e.g. human or animal), for instance: as a protective covering at the ends of long bones (especially at joints); as a structural component of the rib cage, ear, nose, bronchial tubes, intervertebral discs (IVDs); and at many other parts of a subject's body.

The cartilaginous target site suitably comprises a target ECM (suitably as defined herein), ECM cells (suitably as defined herein), and cartilage. In some embodiments, the target ECM itself may be cartilaginous and thus be or comprise cartilage. In some embodiments, the target ECM itself may comprise some or all of the cartilage of the target site. In some embodiments, however, the ECM may not be cartilaginous as such but is suitably surrounded or encapsulated by cartilage. In some embodiments, some or all of the cartilage of the target site may interface with the target ECM (e.g. as per a nucleus pulposus of an IVD, which interfaces with a cartilaginous annular fibrosus and vertebral endplates).

Suitably the cartilage of any cartilaginous target site comprises one or more types of collagen. Most suitably, the cartilage of a cartilaginous target site is or comprises elastic cartilage, hyaline cartilage and/or fibrocartilage.

Elastic cartilage, which is typically present in the outer ear, Eustachian tube and epiglottis, to provide shape and support thereto, comprises elastic fibrous networks, type II as the predominant form of collagen, and elastin as the main protein. In a particular embodiment, the cartilaginous target site(s) comprise elastic cartilage.

Hyaline cartilage, which is most commonly located at articular joints (e.g. covering articular surfaces of bones) as well as at ventral ends of ribs and within the larynx, trachea, and bronchi, comprises significant amounts of type II collagen and chondroitin sulphate and is externally covered by a fibrous membrane of perichondrium or synovium, thereby affording a somewhat less elastic form of cartilage than elastic cartilage. Chondrocytes (cells) are present within the extracellular matrix of hyaline cartilage and contribute to the composition thereof. In a particular embodiment, the cartilaginous target site(s) comprise hyaline cartilage, and suitably in particular articular cartilage.

The target ECM may be the ECM of articular cartilage. Suitably the target ECM is or comprises a middle transitional zone (suitably comprising randomly orientated fibres) and/or a deep zone (suitably with fibres substantially perpendicular to the surface of the articular cartilage) of articular cartilage.

Fibrocartilage, which is found in the pubic symphysis, outer anulus fibrosus (AF) of intervertebral discs, menisci, triangular fibrocartilage temporomandibular joints, as well as at the tendon-bone interface, contains type I collagen as the principle collagen type, sometimes alongside small quantities of type II collagen, and is thus tougher than other forms of cartilage. In a particular embodiment, the cartilaginous target site(s) is or comprises fibrocartilage.

Suitably, the target ECM is part of a core-shell structure, wherein the core is or comprises the target ECM (“ECM core”) and the shell, which is preferably a cartilaginous shell, surrounds and encapsulates the core. The shell itself may be composed of one or more parts, suitably composed of one or more cartilaginous elements (e.g. as per a nucleus pulposus ECM core encapsulated within a shell of annulus fibrosus and vertical endplates). Suitably the shell is a complete shell, suitably without core-exposing defects (e.g. tears, radial fissures, etc. that open a fluid connection between the exterior of the shell and the ECM core). Suitably the shell is sufficiently complete such that the ECM cannot leak out from the shell. Suitably the shell is sufficiently complete such that water cannot leak out from the shell (whether water from the ECM or water injected into the ECM core). The shell structure may be any suitable shell structure, whether a membrane (e.g. basement membrane), a fibrous shell, a cartilaginous shell, or a fibrocartilaginous shell. In a particular embodiment, the shell is a cartilaginous shell comprising a combination of the annulus fibrosus and vertebral endplates of an intervertebral disc, and the ECM core is or comprises a nucleus pulposus (or is or comprises a nucleus pulposus ECM). Suitably the nucleus pulposus is or comprises a nucleus pulposus ECM.

The target ECM is suitably avascular, or substantially avascular. Suitably the target ECM receives nutrients via diffusion, suitably across a surrounding shell or part thereof (e.g. vertebral endplates).

In a particular embodiment, the target site is an intervertebral disc (IVD) or a component part thereof (e.g. nucleus pulposus). Most suitably, the IVD target site is or comprises a target ECM. Though the annulus fibrosus and vertebral end plates each have their own associated extracellular matrices, most suitably the target ECM is the nucleus pulposus ECM (or nucleus pulposus itself, which combines both ECM cells and ECM). The target ECM thus suitably comprises (or has mixed therewith) nucleus pulposus cells (NP cells), suitably selected from chondrocyte-like cells and/or notochordal cells. The nucleus pulposus ECM may be differentiated from the ECMs of the annulus fibrosus and vertebral end plates by methods well known in the art.

In a particular embodiment, the target site is an IVD (or component part thereof, especially the NP) in the lumbar region of a spinal column (i.e. a lumbar vertebrae) or the sacrum region (sacrum vertebrae), though most suitably the lumbar region. Where the target site is within a human subject, the target site is suitably an IVD (or component part thereof, especially the NP) between (and including) the L2 and S1 vertebrae of the spinal column. As such, the target ECM is suitably a nucleus pulposus of one or more vertebrae in the lumbar region.

### Degradation States of Target Sites

Target sites and/or target ECMs can exhibit a range of degradation states or meet a range of degradation state criteria. In the context of the invention, target sites for treatment are suitably first determined by reference to their degradation states, and in particular whether or not their degradation states meet a pre-defined degradation state criteria (i.e. pre-defined candidate degradation state). Most suitably, the determination of degradation states of target sites is useful for determining a candidate subject.

The pre-defined degradation state criteria may be established in a variety of ways known in the art. The pre-defined degradation state criteria may comprise a plurality of criteria, whether weighted or otherwise. In some embodiments, a single criterium may be sufficient.

The pre-defined degradation state criteria may comprise qualitative criteria, quantitative criteria, or a combination of both. A skilled practitioner may be able to determine a degradation state and/or a candidate subject on the basis of qualitative information, for instance by reference to symptoms, images (e.g. MRI images or X-rays, possibly enhanced by the use of contrast media), optionally in combination with additional quantitative data.

Candidate subjects may be identified on the basis of certain inclusion criteria and/or on the basis of certain exclusion criteria. As such, pre-defined degradation state criteria may comprise either or both inclusion criteria and/or exclusion criteria. Inclusion criteria are criteria which, when met, suitably indicate that a given target site or subject comply with the (or a particular) pre-defined degradation state criteria (and thus may be eligible/suitable for receiving treatments according to the invention), whereas exclusion criteria are criteria which, when met, suitably indicate that a given target site or subject is non-compliant with the (or a particular) pre-defined degradation state criteria (and thus may be ineligible/unsuitable for receiving treatments according to the invention). Where the pre-defined degradation state criteria comprise both inclusion criteria and exclusion criteria, suitably if any exclusion criteria are met by the target site(s) or subject, said target site(s) or subject are suitably non-complaint with the (or a particular) pre-defined degradation state criteria (and thus ineligible/unsuitable for receiving treatments according to the invention). Inclusion and exclusion criteria may relate to:
particular conditions (e.g. medical conditions);
particular symptoms (e.g. pain);
particular diagnostic results (e.g. results from diagnostic tests, for instance the results of imaging and the analysis of images obtained therefrom); and/or
particular patient data (e.g. questionnaire data or results obtained therefrom, for instance, Oswestry Disability Index (ODI), EQ-5D questionnaire, VAS questionnaire).

A candidate subject is suitably a subject identified as having one or more target sites exhibiting a degradation state fulfilling or matching one, some, or all of the pre-defined degradation state criteria.

### Inclusion Criteria by Reference to Medical Conditions

The pre-defined degradation state criteria may comprise one or more criteria which correspond with, correlate with (or with a likelihood of the existence of), or are mappable to a particular syndrome or condition.

The particular condition may be a cartilage condition, or degenerated cartilage condition. The cartilage condition may be characterised by partially-degraded cartilage or cartilage with partially-degenerated ECM. In some circumstances, cartilage degradation (e.g. articular cartilage degradation) or damage may be determined, and optionally graded, by arthroscopy. Particular cartilage conditions may include achondroplasia, costochondritis, relapsing polychondritis, spinal disc herniation, or degenerative disc disease (DDD).

The particular condition is suitably early-stage degenerative disc disease (DDD), suitably at one or more particular target sites. A skilled practitioner can readily determine, and optionally assign a grading to, early-stage DDD, for instance, by reference to symptoms (and optionally timings thereof), how the symptoms affect daily activities, other existing medical issues, list of current medications, results of physical examinations (e.g. tests on joints and their range of motion; identification of areas of tenderness, pain, swelling, and/or joint damage; and spinal alignment checks), diagnostic results (joint aspirations, X-rays, MRI scans), and/patient data (e.g. ODI). Such determination tools are discussed further herein.

In a particular embodiment, the condition is early-stage degenerative disc disease, suitably at one or more particular target sites. Early-stage degenerative disc disease is typically characterised as the biological phase of DDD—i.e. a phase during which little structural degradation takes place.

In a particular embodiment, the candidate subject exhibits early-stage degenerative disc disease (DDD) at one or more IVDs.

### Exclusion Criteria by Reference to Medical Conditions

The pre-defined degradation state criteria may comprise one or more criteria which correspond with, correlate with (or with a likelihood of the existence of), or are mappable to a particular syndrome or condition which, if exhibited, indicates non-compliance with the (or a particular) pre-defined degradation state criteria. Suitably excluded conditions include spinal-conditions which are not DDD—e.g. a subject exhibiting spinal conditions other than DDD may not meet the pre-defined degradation state criteria.

For example, a particular condition excluded by the pre-defined criteria may be late-stage degenerative disc disease (DDD), suitably at one or more relevant target sites under consideration. As per corresponding inclusion criteria, a determination (and optionally grading) of late-stage DDD may be established by a skilled practitioner. In a particular embodiment, a particular condition excluded by the pre-defined criteria may include degenerative disc disease (DDD), suitably at one or more target sites, wherein substantial structural damage or deformation (bone spurs, ligament ossification) has occurred at the one or more target sites.

Other suitable excluded conditions (i.e. conditions which a candidate subject should not have, at least not at one or more relevant target sites under consideration) may comprise osteoarthritis, Lytic Spondylolisthesis and/or Degenerative Spondylolisthesis>grade I Meyerding. Suitably the candidate should not have Lytic Spondylolisthesis and/or Degenerative Spondylolisthesis>grade I Meyerding.

Other exclusions may suitably include patients who have already undergone surgical procedures at the pertinent target site, for instance a partial or total discectomy or any annulotomy.

Other exclusions may suitably include patients exhibiting physical deficits at end plates, in particular Schnorl nodules.

### Inclusion Criteria by Reference to Symptoms

The pre-defined degradation state criteria may comprise one or more criteria which correspond with, correlate with (or with a likelihood of the existence of), or are mappable to a particular symptom or set of symptoms. Such symptoms may themselves be mappable to a particular condition, for instance, early-stage degenerative disc disease (DDD).

Such symptoms may include pain or a particular type of pain (which a skilled practitioner could suitably characterise), for instance, discogenic pain. Discogenic pain may be characterised by pain in the center or slightly lateral to the midline in the lower lumbar region or pain referred to the buttock and posterior thigh with prolonged sitting or flexion. The symptoms may include discogenic lower back pain, suitably confirmed by a history of Lower Back Pain, with a minimum of 3 months of continuous pain or 6 months of acute episodes of pain. Suitably the discogenic pain stems from one or more IVDs in the lumbar region of the spine, suitably between the L2 and S1 vertebrae where the subject is a human subject.

The symptoms may include chronic lower back pain (CLBP).

In a particular embodiment, the candidate subject exhibits symptoms of discogenic pain at one or more IVDs.

### Exclusion Criteria by Reference to Symptoms

The pre-defined degradation state criteria may comprise one or more criteria which correspond with, correlate with (or with a likelihood of the existence of), or are mappable to a particular symptom or set of symptoms which, if exhibited, indicates non-compliance with the (or a particular) pre-defined degradation state criteria. Suitably, excluded symptoms may comprise pain, pain types, or particular sources of pain. Suitably, excluded symptoms may comprise pain or pain types other than a pain or pain type defined in respect of symptom inclusion criteria (e.g. spinal-derived pain other than discogenic pain). By way of example, excluded symptoms may comprise non-discogenic pain. Excluded symptoms may suitably comprise primary root pain or facet pain.

Suitably, excluded pain symptoms comprise pain whose source is innervated structures within the spinal column (facet joints, endplate), neural elements within the spinal canal (nerve radicles, cauda equina), extraspinal disease (genitourinary, vascular, gastrointestinal, myofascial sacroiliac), radiculopathy, and/or sciatica.

### Inclusion Criteria by Reference to Diagnostic Results

The pre-defined degradation state criteria may comprise one or more criteria which correspond with, correlate with (or with a likelihood of the existence of), or are mappable to one or more particular diagnostic results (“diagnostic results criteria”). Such diagnostic result(s) may themselves be mappable to a particular condition, for instance, early-stage degenerative disc disease (DDD). Suitably the one or more diagnostic results are obtained, obtainable, derived, or derivable via one or more corresponding diagnostic tests.

The diagnostic results criteria may comprise a grading or a classification in respect of one or more target sites. Several grading and classification schemes are used by skilled practitioners to establish degradation states of target sites. For example, cartilage degeneration (e.g. articular cartilage degradation) at a particular cartilaginous target site may be graded in accordance with the Outerbridge Classification (International Cartilage Repair Society) following an arthroscopy. In such a case, diagnostic results criteria may include characterisations of target sites at grade I, II, or III according to the Outerbridge Classification (International Cartilage Repair Society), more suitably grade I or II. Relevant diagnostic results criteria in respect of the degeneration of IVDs may include gradings in accordance with the Pfirrmann classification. In such a case, diagnostic results criteria may include characterisations of IVD target sites at grade II or III according to the Pfirrmann classification system, more suitably grade II.

Suitably at least one of the one or more diagnostic result(s) is obtained or obtainable by imaging one or more target sites, suitably radiographic imaging, most suitably magnetic resonance imaging (MRI). Suitably the diagnostic result(s) derived/derivable from said imaging, especially in the case of MR imaging, comprise one or more of images, imaging data, time-course data (e.g. relaxation times), and other such data derived/derivable through processing any one or more of the aforesaid.

Magnetic resonance imaging (MRI) is particularly useful for assessing degeneration and/or damage in respect of cartilaginous elements of the body, such as articular joints and intervertebral discs. The Pfirrmann classification of disc degeneration utilises T2-weighted MR images of IVDs in the grading of disc degeneration. The Pfirrmann system utilises signal intensities and morphology to rank disk degeneration according to fives grades. The Pfirrmann grading system (and methods of grading) is defined in detail in Pfirrmann et al, “Magnetic resonance classification of lumbar intervertebral disc degeneration”, Spine. 2001; 26 (17): 1873-8, which is incorporated herein by reference. The five grades may be broadly defined as follows:
1. Grade I: homogeneous disc with bright hyperintense white signal intensity and normal disc height;
2. Grade II: inhomogeneous disc, but maintains the hyperintense white signal; nucleus and annulus still clearly differentiated, optionally exhibiting a gray horizontal band; substantially normal disc height;
3. Grade III: inhomogeneous disc with an intermittent gray signal intensity; unclear distinction between nucleus and annulus; disc height is normal or slightly decreased;
4. Grade IV: inhomogeneous disc with a hypointense dark gray signal intensity; no distinction between the nucleus and annulus; disc height is slightly or moderately decreased;
5. Grade V: inhomogeneous disc with a hypointense black signal intensity; no difference between the nucleus and annulus; the disc space is collapsed.

The diagnostic results criteria suitably comprise a Pfirrmann grading (or range of Pfirrmann gradings), suitably in respect of one or more IVD target sites. The diagnostic results criteria suitably comprise one or more IVD target sites being designated as Grade II or III on the Pfirrmann scale. Thus IVD target sites designated Grade II or III on the Pfirrmann scale may be suitably indicative of early-stage DDD. In a particular embodiment, the one or more target sites exhibiting a required degradation stage are one or more IVDs designated as Grade II or Grade III on the Pfirrmann scale, most suitably Grade II but optionally also Grade III where an IVD complies with the certain additional qualifying criteria relating to the exposure of (or leakability of) the nucleus pulposus—see below.

Grade III-graded IVDs may comprise radial fissures and/or other defects in the annulus fibrosus and/or vertebral endplates. However, where the diagnostic results criteria include a Grade III Pfirrmann grading (as well as or instead of Grade II), such criteria is/are further qualified by a requirement that the nucleus pulposus (i.e. core of the IVD) of the relevant one or more IVD target sites is not exposed and/or cannot leak out of the IVD. Suitably, where the diagnostic results criteria include this Grade III Pfirrmann grading, such criteria is/are further qualified in that the annulus fibrosus and vertebral endplates of a Grade III IVD target site are free from defects (e.g. especially radial fissures) that open a path (or fluid connection) between the nucleus pulposus core of the IVD and the exterior of the IVD. Thus, suitably Grade III IVD target sites must comprise a fully-contained nucleus pulposus core to comply with the diagnostic results criteria. By definition, suitably these additional qualifying requirements for Grade III IVDs apply also to Grade II IVDs (though Grade II IVDs generally do not exhibit the same structural defects).

Suitably, wherever fissures and/or other defects are present in the annulus fibrosus, such fissures and/or other defects do not violate the outer annulus. Suitably, wherever fissures and/or other defects are present in the annulus fibrosus, such fissures and/or other defects do not create a continuous path between the centre of the disc (where the native nucleus pulposus and/or post-treatment compositions of the invention reside) and the outside of the disc and/or the spinal canal space. Verification of the nature of any such fissures and/or other defects, whether suspected or otherwise, may suitably be obtained through contrast medial visualisation(s).

Other similar scales and techniques may be deployed to determine a degradation state of a target site, and as such the diagnostic results criteria may comprise definitions according to such other scales and techniques instead of or in addition to any of the aforesaid. For instance, the Griffith Scale (Griffith J F, Wang Y X, Antonio G E et-al. Modified Pfirrmann grading system for lumbar intervertebral disc degeneration. Spine. 2007; 32 (24): E708-12) is an adaptation of the Pfirrmann Scale involve 8 grades rather than 5, but this can still be used to establish early-stage DDD, especially early-stage DDD with the additional qualification requirements mentioned above in relation to the exposure of the nucleus pulposus core.

Suitably, the diagnostic results criteria may additionally comprise a requirement for a substantially homogenous distribution of the trabeculae in the vertebral body below a particular IVD target site in question (suitably with substantially no disturbance in the load distribution). Suitably, additional diagnostic results criteria may accommodate a mild disturbance in the load distribution including, for instance, a less dense centre and slight lateral reinforcement as may be observed in the case of dehydration of the nucleus pulposus but substantially no inflammation surrounding the endplate.

Newer imaging techniques, such as T2 mapping, diffusion imaging, T1p mapping, MR spectroscopy, and nuclear imaging may also be deployed to determine early degeneration stages (e.g. early-stage DDD) for revitalising clinical interventions. Such techniques are described further in the Background section of the present specification.

### Exclusion Criteria by Reference to Diagnostic Results

The pre-defined degradation state criteria may comprise one or more criteria which correspond with, correlate with (or with a likelihood of the existence of), or are mappable to one or more particular diagnostic results which, if exhibited, indicates non-compliance with the (or a particular) pre-defined degradation state criteria (“diagnostic results exclusion criteria”). Such excluded diagnostic result(s) may themselves be mappable to a particular condition, for instance, non-early-stage degenerative disc disease (DDD). Suitably the one or more diagnostic results are obtained, obtainable, derived, or derivable via one or more corresponding diagnostic tests.

The diagnostic results exclusion criteria may comprise a grading or a classification in respect of one or more target sites, suitably one which is different from any defined in accordance with inclusion criteria. For example, for cartilage degeneration (e.g. articular cartilage degradation) at a particular cartilaginous target site, diagnostic results exclusion criteria may include characterisations of target sites at grades other than grade I, II, or III according to the Outerbridge Classification (International Cartilage Repair Society), more suitably grades other than grade I or II. Relevant diagnostic results exclusion criteria in respect of the degeneration of IVDs may include characterisations of IVD target sites at grades other than grade II or III according to the Pfirrmann classification system, more suitably grades other than grade II. In a particular embodiment, especially where an inclusion criteria includes IVD target sites designated grade II or III on the Pfirrmann scale, the diagnostic results exclusion criteria includes grade III IVDs whose annulus fibrosus exhibit any radial fissures, more suitably grade III IVDs which exhibit radial fissures that expose the nucleus pulposus core to the exterior of the particular grade III IVD.

The diagnostic results exclusion criteria may comprise the presence of defects, tears, or fissures (particularly radial fissures) at the exterior surface of an annulus fibrosus or vertebral endplate(s).

The diagnostic results exclusion criteria may comprise the presence of posterior bone spurs (osteophytes), especially in the context of IVDs.

The diagnostic results exclusion criteria may comprise a Modic signal grade 1 at the relevant IVD target site/disc level.

The diagnostic results exclusion criteria may comprise a vertebral endplate defects or weakness, for example the presence of a Schmorl nodule.

The diagnostic results exclusion criteria may comprise disc collapse greater than or equal to 15% of the original disc height (determinable by comparison with the height of an upper adjacent disc).

The diagnostic results exclusion criteria may comprise a bulging disc, whether confirmed by protrusion, herniation or prolapse of the vertebral disc from its normal position in the vertebral column.²¹

The diagnostic results exclusion criteria may comprise congenital or idiopathic and degenerative deformities of the spine (e.g. Scoliosis>20° cobb angle).

The diagnostic results exclusion criteria may comprise old or acute vertebral fractures.

### Inclusion Criteria by Reference to Patient Data

The pre-defined degradation state criteria may comprise one or more criteria which correspond with, correlate with (or with a likelihood of the existence of), or are mappable to one or more particular patient data (“patient data criteria”). Such patient data criteria may be mappable to a particular condition, for instance, early-stage degenerative disc disease (DDD). Suitably patient data criteria is derived from or is in relation to a potential candidate subject.

The patient data criteria may, for example, comprise a score in accordance with the Oswestry Disability Index (ODI) (Fairbank J C, Pynsent P B. The Oswestry Disability Index. Spine 2000 Nov. 15; 25 (22): 2940-52). ODI is an index which can be determined from the Oswestry Low Back Pain Questionnaire, which is commonly used by clinicians and researchers to quantify disability for low back pain, the current version of which is described in the aforementioned Fairbank et al paper. The questionnaire in question is completed by a potential candidate subject, and covers various topics such as intensity of pain, lifting, ability to care for oneself, ability to walk, ability to sit, sexual function, ability to stand, social life, sleep quality, and ability to travel. The ultimate scores obtained from the questionnaire provide an index between 0 and 100, where 0 means no disability and 100 means maximum disability. The ODI scores broadly map as follows:
0 to 20: Minimal disability
21-40: Moderate Disability
41-60: Severe Disability
61-80: Crippling back pain
81-100: These patients are either bed-bound or have an exaggeration of their symptoms.

The patient data criteria may suitably comprise an ODI greater than or equal to 10, suitably greater than or equal to 15, suitably greater than or equal to 20, suitably greater than or equal to 30, suitably greater than or equal to 40, suitably greater than or equal to 50. The patient data criteria may suitably comprise an ODI less than or equal to 95, suitably less than or equal to 90, suitably less than or equal to 80, suitably less than or equal to 70, suitably less than or equal to 60, suitably less than or equal to 50, suitably less than or equal to 40, suitably less than or equal to 30. In a particular embodiment, the patient data criteria comprises an ODI between 10 and 70, suitably between 15 and 65, most suitably between 20 and 60.

The patient data criteria may suitably comprise (especially where the patient is a human) being aged greater than or equal to 10, suitably greater than or equal to 15, suitably greater than or equal to 18, suitably greater than or equal to 25, suitably greater than or equal to 30. The patient data criteria may suitably comprise being aged less than or equal to 80, suitably less than or equal to 70, suitably less than or equal to 60, suitably less than or equal to 55, suitably less than or equal to 50, suitably less than or equal to 40. In a particular embodiment, the patient data criteria comprises being aged between 10 and 90, suitably between 18 and 55, suitably between 25 and 50.

### Exclusion Criteria by Reference to Patient Data

The pre-defined degradation state criteria may comprise one or more criteria which correspond with, correlate with (or with a likelihood of the existence of), or are mappable to one or more particular patient data which, if exhibited, indicates non-compliance with the (or a particular) pre-defined degradation state criteria (“patient data exclusion criteria”). The patient data exclusion criteria may comprise one or more criteria defined by being differ or other than any of the patient data criteria defined in relation to inclusion criteria.

### Particular Embodiments

In a particular embodiment, the target site is an IVD whose degradation state corresponds with early-stage degenerative disc disease where the nucleus pulposus of the IVD is fully contained by its corresponding annulus fibrosus and vertebral endplate.

In a particular embodiment, the target site is an IVD whose nucleus pulposus comprises internal defects, suitably characterizable as tears, clefts, or fissures.

In a particular embodiment, the target site is a cartilaginous target site (e.g. an IVD) comprising a target ECM with ECM cells dispersed therein, wherein the target ECM comprises at least 60 wt % water, suitably at least 70 wt % water.

In a particular embodiment, the target site is an IVD whose degradation state corresponds with early-stage DDD, wherein the disc-height of the IVD has collapsed by no more than 15%, suitably by no more than 10%, more suitably by no more than 5%. In such embodiments, suitably substantially no osteoarthritis exists at the IVD and the IVD does not exhibit Lytic Spondylolisthesis and/or Degenerative Spondylolisthesis>grade I Meyerding.

In a particular embodiment, the target site is an IVD, suitably in a lumbar region of a subject's spine, whose degradation state is characterised by symptoms of discogenic pain, which discogenic pain has been suitably continuous for at least 3 months. In such embodiments, most suitably the target site does not exhibit non-discogenic pain, in particular pain whose source is either innervated structures within the spine, neural elements within the spine, extraspinal disease, radiculopathy, or sciatica.

In a particular embodiment, the target site is an IVD, suitably in a lumbar region of a subject's spine, whose degradation state is characterised by symptoms of discogenic pain (and suitably not other types of pain) and early-stage DDD wherein the disc-height of the IVD has collapsed by no more than 15%, suitably by no more than 10%, more suitably by no more than 5%.

In a particular embodiment, the target site is an IVD whose degradation state is determinable from degradability data comprising a Pfirrmann grading for the IVD. In such an embodiment, the pre-defined degradation state (used, for instance, for identifying a partially-degraded target site and/or a candidate subject) suitably comprises a Pfirrmann grading of grade II or grade III, though preferably to comply with the pre-defined degradation state any grade III IVDs comprise a fully-contained nucleus pulposus.

In a particular embodiment, the target site is an IVD, suitably in the lumbar region of a subject's spine, whose degradation state is characterised by early-stage DDD wherein the disc-height of the IVD has collapsed by no more than 15%, suitably by no more than 10%, more suitably by no more than 5%, wherein early-stage DDD is confirmed by a Pfirrmann grading of grade II or grade III, though preferably to comply with the pre-defined degradation state any grade III IVDs comprise a fully-contained nucleus pulposus.

In a particular embodiment, the target site is an IVD, suitably in the lumbar region of a subject's spine, whose degradation state is characterised by early-stage DDD determined by radiographic imaging, suitably via MRI, in particular by reference to T2-weighted MR images.

In a particular embodiment, the target site is an IVD, suitably in the lumbar region of a subject's spine, whose degradation state is characterised by an ODI between 20 and 60, and a Pfirrmann grading of grade II or grade III, though preferably to comply with the pre-defined degradation state any grade III IVDs comprise a fully-contained nucleus pulposus, wherein the disc-height of the IVD has collapsed by no more than 15%, suitably by no more than 10%, more suitably by no more than 5%.

In all of the aforementioned embodiments involving an IVD as a target site, suitably the IVD has a nucleus pulposus that is clearly distinguishable from the annulus fibrosus, and wherein the annulus fibrosus and vertebral endplates are substantially healthy and without defects, tears, or fissures.

### Identifying Candidate Subjects

Suitably a candidate subject (i.e. a human or animal subject identified as in need of a treatment of the present invention) is identified on the basis of whether the subject comprises one or more target sites exhibiting (or having a degradation state complying with) characteristics of a partially-degraded target site as defined herein (or a pre-defined degradation state/criteria as defined herein).

Suitably, a candidate subject comprises one or more partially-degraded target sites exhibiting a degradation state complying with one or more pre-defined degradation state criteria as defined herein, wherein the degradation state criteria may suitably be selected from any one or more inclusion and/or exclusion criteria as defined herein.

Suitably, a candidate subject may be determined by grading one or more target sites in accordance with an appropriate grading scale (e.g. Pfirrmann scale) and deducing whether any of the target site(s) exhibit a grade complying with one or more inclusion and/or exclusion criteria as defined herein.

In a particular embodiment, a candidate subject has one or more target sites complying with any one or more of the aforementioned embodiments relating to a target site. For instance, a candidate subject may suitably have one or more IVDs, suitably in the lumbar region of the subject's spine, whose degradation state is characterised by an ODI between 20 and 60, a disc-height which has collapsed by no more than 15%, suitably by no more than 10%, more suitably by no more than 5%, and a Pfirrmann grading of grade II or grade III, though preferably any Pfirrmann-graded III IVDs comprise a fully-contained nucleus pulposus.

Since a candidate subject (or candidate patient) can be determined by reference to degradation state(s) of target site(s) therein, any method of determining the candidate subject may be a method of diagnosis or else a method involving determining whether the target site(s) or their respective degradation state(s) conform to a pre-defined candidate degradation state.

Any method of diagnosis may exclude method steps performed upon a human or animal body, and may, for instance, be confined to steps of examining and/or processing degradation data (e.g. MRI images) relating a one or more target sites of the subject, and deriving a diagnosis therefrom (e.g. by comparison with a pre-determined criteria). However, methods of diagnosis may involve an act of obtaining degradation data from a subject (e.g. obtaining MRI images, obtaining an ODI).

Suitably a candidate subject has (or is determined to have, suitably via any one or more techniques described herein) degenerative disc disease in respect of one or more IVDs. Most suitably a candidate subject has (or is determined to have, suitably via any one or more techniques described herein) early-stage degenerative disc disease in respect of one or more IVDs, suitably in a lumbar region of the subject's spine. Most suitably a candidate subject experiences symptoms of discogenic pain in respect of the one or more target IVDs.

The candidate subject is suitably any animal or human subject. Animal subjects may, for instance, include farm animals such as goats, cows, sheep, pigs, horses, and may include pet animals, for instance, dogs and cats. Most suitably, however, the candidate subject is a human subject.

### Treatments of Candidate Subjects

Having established a candidate subject, the present invention may comprise treating the candidate subject, or treating one or more partially-degraded target sites identified therein.

Treatments of the invention suitably involve administering a treatment composition (or hydrogel composition) to one or more partially-degraded target sites of a subject. In a particular embodiment, for instance, a treatment of the invention may involve injection of a liquid-phase treatment composition into a nucleus pulposus (or ECM thereof) of one or more partially-degraded IVD target sites of a candidate subject, whereafter the treatment composition cures in situ to form a hydrogel and thereby revitalise the partially-degraded IVD target site(s).

Most suitably, compositions of the invention may be categorised as treatment compositions and post-treatment (hydrogel) compositions, where a treatment composition is the composition to be administered and the post-treatment composition is the composition following its administration to a target site. The treatment composition and post-treatment composition may differ in one or more respects, suitably either or both physically and/or chemically.

### Treatment Compositions and Post-Treatment (Hydrogel) Compositions

According to the invention, a candidate subject may suitably be treated with a treatment composition, which is suitably administered to or introduced into a target site (or ECM thereof) of the candidate subject, most suitably by injection. The treatment composition suitably comprises one or more gellable components (or precursors thereto). Herein, a “gellable” component, material, or composition is suitably one that may be caused to gel if it is not already in a gelled state. A precursor to a gellable component, material, or composition, is suitably one which may be transformed in some manner, suitably chemically (e.g. through formation of new covalent bonds), into the gellable component, material, or composition.

The treatment composition may be formed prior to, during, and/or after administration, but most suitably the treatment composition is an administrable composition (i.e. a pre-administration composition) or an administrable form of a hydrogel composition (i.e. post-treatment composition).

The treatment composition most suitably refers to the composition as it exists before actual introduction into the target site, though the treatment composition may itself be formed during the administration process (e.g. by pre-mixing compositions to form the treatment composition, for instance using a double-barreled syringe and a mixing chamber). Moreover, the treatment composition may exist (especially during administration/introduction steps) in a state of physical and/or chemical flux—for instance the composition may be undergoing a physical transformation (e.g. swelling) and/or undergoing a chemical transformation (e.g. as a result of one or more chemical reactions). In some embodiments, the treatment composition is substantially physically and/or chemically stable (i.e. not in a state of physical and/or chemical flux, save for any degradation). The treatment composition may be one of a plurality of compositions which are mixed before or during administration, though suitably in this context the treatment composition comprises one, some, or all of the active ingredients (e.g. gellable components or precursors thereto).

The treatment composition suitably is, becomes, or is otherwise transformed into a hydrogel composition at the target site (i.e. following introduction thereto), though any transformation may be non-instantaneous and may occur over time (though suitably within 7 days, suitably within 48 hours, suitably within 24 hours, suitably within 12 hours, suitably within 2 hours, suitably within 30 mins, suitably within 10 mins, suitably within 2 mins). As such, the hydrogel composition may be termed a “post-treatment composition”.

The post-treatment composition is suitably physically different to the treatment composition, suitably physically stronger (as, for instance, measurable by a Young's modulus) than the treatment composition, suitably less fluid (or of a higher viscosity) than the treatment composition, suitably more gelled than the treatment composition. Suitably the post-treatment composition is a gel, suitably a hydrogel, whereas the treatment composition is suitably a non-gelatinous fluid. Most suitably the treatment composition is physically transformed (instantaneously or gradually) at the target site and/or en route thereto.

In some embodiments, the post-treatment composition may be substantially chemically identical to the treatment composition (e.g. relevant components undergo substantially no molecular changes, at least in terms of inter- and intra-molecular non-ionisable covalent bonds). In some embodiments, components of the treatment composition remain substantially chemically identical (suitably at least with respect to inter- and intra-molecular non-ionisable covalent bonds within or between said components) within the post-treatment composition. Changes in ionisation, protonation, and/or ion associations of components may be expected between the treatment composition and post-treatment composition (e.g. in accordance with local pH-changes).

The post-treatment composition may comprise additional component(s) to the original treatment composition as a result of mixing with such additional component(s) at or around the target site (this may include changes in dilution level, for instance, as a result of further swelling in situ).

In some embodiments, the post-treatment composition may be chemically different to the treatment composition (e.g. relevant components undergo molecular changes, at least in terms of inter- and intra-molecular non-ionisable covalent bonds). In some embodiments, one or more components of the treatment composition are chemically changed/different (suitably at least with respect to inter- and intra-molecular non-ionisable covalent bonds within or between said components) within the post-treatment composition. Such molecular changes are suitably a result of inter-molecular chemical reactions, suitably resulting in the formation of new inter-molecular covalent bonds. As such, suitably the treatment composition comprises one or more reactive components. Said components may react as a consequence of prevailing conditions at a target site or may react as a consequence of a reactant or initiator being introduced to the treatment composition, suitably prior to introduction of the treatment composition to the target site.

Suitably the treatment composition is a fluid composition, suitably a mobile fluid. Suitably the treatment composition is fluid, suitably a liquid composition or a liquid dispersion, emulsion, and/or suspension composition. Suitably, in contrast, the post-treatment composition is non-fluid, suitably non-mobile, suitably non-free-flowing. Suitably the treatment composition is a non-gelled fluid whereas the post-treatment composition is a gelled composition (e.g. a hydrogel).

Suitably the treatment composition is deliverable to a subject via injection, suitably via an outlet (e.g. syringe needle or cannula) whose largest inner dimension (or inner diameter) is less than or equal to 2 mm, suitably less than or equal to 1.6 mm, suitably less than or equal to 1.4 mm, suitably less than or equal to 1.2 mm; and suitably whose largest inner dimension (or inner diameter) is greater than or equal to 0.2 mm, suitably greater than or equal to 0.4 mm, suitably greater than or equal to 0.5 mm. Suitably, the outlet, syringe needle, or cannula corresponds to a Birmingham Gauge between G12 and G 25, suitably between G14 and G23, most suitably between G16 and G21. Suitably the post-treatment composition is not deliverable under the same conditions and using them same equipment—for instance, the post-treatment composition is suitably too viscous or too immobile to exit an outlet of the syringe.

Suitably, the treatment composition and, where it is different, the post-treatment composition is biocompatible, and suitably non-toxic (especially non-toxic to any ECM cells).

Suitably the post-treatment composition is (substantially) non-biodegradable. Suitably the post-treatment composition is incapable of enzymatic degradation, especially via enzymes present at the target site. Suitably the post-treatment composition is substantially physically stable at/in the target site, suitably for at least 6 months, suitably for at least 9 months, suitably for at least 1 year, suitably for at least 2 years, suitably for at least 5 years, suitably for at least 10 years. Suitably the post-treatment composition is substantially chemically and/or biochemically stable at/in the target site, suitably for at least 6 months, suitably for at least 9 months, suitably for at least 1 year, suitably for at least 2 years, suitably for at least 5 years, suitably for at least 10 years. Suitably the post-treatment composition can remain immobilised within the target site for at least 6 months, suitably for at least 9 months, suitably for at least 1 year, suitably for at least 2 years, suitably for at least 5 years, suitably for at least 10 years.

The post-treatment composition suitably mimics the target site (especially a relevant ECM). Suitably the post-treatment composition mimics the target site (or ECM thereof) in terms of physical properties or physical form. Suitably the post-treatment composition mimics the target site (or ECM thereof) in terms of biomechanical properties. Suitably the post-treatment composition is chemically distinct from the target site (or ECM thereof). Suitably the physical similarity of the post-treatment composition to the target site (or ECM thereof) contributes to one or more treatment effects (e.g. healing, cell-revitalisation).

Suitably the volume of treatment composition and/or post-treatment composition delivered to the target site is at or below 3 mL, more suitably at or below 2 mL, suitably at or below 1.5 mL, suitably at or below 1 mL. Where the target site is an IVD, suitably the disc height is increased by no more than 30%, suitably by no more than 20%, suitably by no more than 15%, suitably by no more than 10%, following deliver of the treatment composition and/or post-treatment composition to the IVD or nucleus pulposus thereof.

The treatment composition suitably comprises one or more active components (or one or more active precursor components, i.e. precursor(s) which may transform into active component(s) before, during or after administration), where the active component is suitably “active” in the sense that it is responsible for eliciting an effect (e.g. healing effect and/or cellular response) at the target site. As such, the post-treatment composition suitably comprises one or more active components, most suitably one active component. The active component(s) are suitably gellable or gelled components, suitably hydrogels.

Most suitably, the treatment composition comprises an active precursor component and the post-treatment composition comprises a corresponding active component, wherein the active component is suitably derived from the active precursor component, suitably by virtue of either or both a physical and/or a chemical change. Suitably the active precursor component of the treatment composition is transformed into the active component of the post-treatment composition, suitably by virtue of polymerisation (or crosslinking) of the active precursor component to form an active component that is a polymer derived from monomeric active precursor components. Suitably such polymerisation (or crosslinking) is free-radical polymerisation (or crosslinking). The treatment composition suitably comprises 1-30 wt % active precursor component (and suitably the corresponding post-treatment composition comprises 1-30 wt % active component), suitably 5-25 wt % active precursor component (and suitably the corresponding post-treatment composition comprises 5-25 wt % active component), suitably 10-20 wt % active precursor component (and suitably the corresponding post-treatment composition comprises 10-20 wt % active component), more suitably 12-18 wt % active precursor component (and suitably the corresponding post-treatment composition comprises 12-18 wt % active component), most suitably about 14 wt % active precursor component (and suitably the corresponding post-treatment composition comprises about 14 wt % active component). Suitably a treatment composition comprises 1 to 60 wt % gellable particles (suitably microgel particles, suitably microgel particles with pre-grafted vinyl groups), suitably 2 to 30 wt %, suitably 5 to 20 wt %, suitably 10 to 20 wt %, suitably 13-17 wt %, suitably about 14 wt %.

The treatment composition may additionally comprise one or more activator agents, wherein said activator agent(s) promote transformation of the active precursor component(s) into the active component(s), be it physical transformation, chemical transformation, or a combination thereof.

The activator agent(s) suitably comprise one or more physical activator agents and/or one or more chemical activator agents. A physical activator agent suitably promotes a physical transformation of the active precursor component(s). A chemical activator agent suitably promotes a chemical transformation of the active precursor component(s).

The treatment composition suitably comprises a chemical activator agent, which is suitably a transformation reagent or initiator which suitably promotes (triggers and/or participates in) chemical transformation of the active precursor component(s) into the active component(s). Where the transformation is characterised by polymerisation (or crosslinking) of the active precursor component to form an active component, the transformation reagent may be a polymerisation initiator, most suitably a free-radical initiator. The transformation reagent (or reaction products derived therefrom) may be present within the post-treatment composition. Suitably, therefore, the transformation reagent and/or reaction products derived therefrom a substantially non-toxic, especially to cells located within or around the target site. In a particular embodiment, the treatment composition comprises at least two chemical activator agents, one being an initiator and the other being an accelerator (suitably which partners the initiator).

The treatment composition suitably comprises 0.001-6 wt % chemical activator agent(s) (and suitably the corresponding post-treatment composition comprises 0.001-6 wt % chemical activator agent(s) or products derived therefrom), suitably 0.01-3 wt % chemical activator agent(s) (and suitably the corresponding post-treatment composition comprises 0.01-3 wt % chemical activator agent(s) or products derived therefrom), suitably 0.1-1 wt % chemical activator agent(s) (and suitably the corresponding post-treatment composition comprises 0.1-1 wt % chemical activator agent(s) or products derived therefrom), suitably 0.2-0.5 wt % chemical activator agent(s) (and suitably the corresponding post-treatment composition comprises 0.2-0.5 wt % chemical activator agent(s) or products derived therefrom), suitably 0.25-0.38 wt % chemical activator agent(s) (and suitably the corresponding post-treatment composition comprises 0.25-0.38 wt % chemical activator agent(s) or products derived therefrom).

The treatment composition suitably comprises 0.001-5 wt % initiator(s) (and suitably the corresponding post-treatment composition comprises 0.001-5 wt % initiator(s) or products derived therefrom), suitably 0.01-3 wt % initiator(s) (and suitably the corresponding post-treatment composition comprises 0.01-3 wt % initiator(s) or products derived therefrom), suitably 0.1-1 wt % initiator(s) (and suitably the corresponding post-treatment composition comprises 0.1-1 wt % initiator(s) or products derived therefrom), suitably 0.2-0.4 wt % initiator(s) (and suitably the corresponding post-treatment composition comprises 0.2-0.4 wt % initiator(s) or products derived therefrom), suitably 0.25-0.3 wt % initiator(s) (and suitably the corresponding post-treatment composition comprises 0.25-0.3 wt % initiator(s) or products derived therefrom) . . .

The treatment composition suitably comprises 0.0001-2 wt % accelerator(s) (and suitably the corresponding post-treatment composition comprises 0.0001-2 wt % accelerator(s) or products derived therefrom), suitably 0.001-1 wt % accelerator(s) (and suitably the corresponding post-treatment composition comprises 0.001-1 wt % accelerator(s) or products derived therefrom), suitably 0.01-0.5 wt % accelerator(s) (and suitably the corresponding post-treatment composition comprises 0.01-0.5 wt % accelerator(s) or products derived therefrom), suitably 0.05-0.15 wt % accelerator(s) (and suitably the corresponding post-treatment composition comprises 0.05-0.15 wt % accelerator(s) or products derived therefrom), suitably 0.07-0.1 wt % accelerator(s) (and suitably the corresponding post-treatment composition comprises 0.7-0.1 wt % accelerator(s) or products derived therefrom).

The treatment composition suitably comprises a physical activator agent, which is suitably a pH-modifier and/or buffer. The pH-modifier may be present at a concentration that causes the treatment composition (or active component or active precursor component thereof) to swell or gel (or to begin swelling or gelling).

The treatment composition suitably comprises sufficient of physical activator agent(s) (suitably pH-modifier(s) and/or buffer(s)) to furnish the treatment composition (and suitably also the post-treatment composition) with a pH between 4 and 12, suitably a pH at or above 5, suitably at or above 6, most suitably at or above 7, suitably at or below 12, suitably at or below 10, suitably at or below 9, suitably at or below 8. Depending on the physical activator agent(s) in question, only small quantities may be required. Suitably the pH of the treatment composition is between pH 7 and 8, most suitably about pH 7.4.

In a particular embodiment, the treatment composition comprises both a physical activator and a chemical activator, and suitably chemical activation (e.g. polymerisation) cannot occur without physical activation (e.g. swelling).

The treatment composition may additionally comprise a contrast or visualisation agent, for instance to assist administration of the treatment composition to a target site (e.g. where image-guided administration is involved) and/or to assist in monitoring the fate of the post-treatment composition following administration. In an embodiment, the contrast agent is radio-opaque. In an embodiment, the contrast agent is barium sulphate (BaSO₄). Suitably the contrast agent (especially there it is BaSO₄) is present within the treatment composition at a concentration of 0.1-20 wt %, more suitably 1-10 wt %, more suitably 3-8 wt %, more suitably 5-7 wt %.

Suitably, the treatment composition (and suitably also the post-treatment composition) comprise at least 50 wt % water, suitably at least 60 wt % water, suitably at least 70 wt % water, suitably at least 75 wt % water, suitably at least 80 wt % water, suitably at least 85 wt % water, suitably at least 90 wt % water. Suitably, the treatment composition (and suitably also the post-treatment composition) comprise at most 99 wt % water, suitably at most 95 wt % water, suitably at most 90 wt % water, suitably at most 85 wt % water. Suitably, the treatment composition (and suitably also the post-treatment composition) comprise sufficient water to physically-mimic a healthy target site or healthy target ECM. Suitably, the treatment composition (and suitably also the post-treatment composition) comprises 50-95 wt % water, more suitably 70-85 wt % water, suitably 80-85 wt % water.

In a particular embodiment, the treatment composition comprises, or is formed by mixing together, an activatable composition (comprising an active precursor component) and an activator composition (comprising one or more activator agents, suitably which activate the activatable composition, for instance, by promoting conditions required for the transformation of the active precursor component into the active component).

Suitably, the treatment composition (and suitably therefore any compositions used to prepare the treatment composition) are substantially free from proteins, suitably substantially free from any GAG-bearing proteins (e.g. proteoglycans).

Suitably, the treatment composition (and suitably therefore any compositions used to prepare the treatment composition) are substantially free from any growth factors.

Suitably, the treatment composition (and suitably therefore any compositions used to prepare the treatment composition) are substantially free from any cells, cellular materials, or extracellular materials.

Suitably, the treatment composition (and suitably therefore any compositions used to prepare the treatment composition) are substantially free from any silicon-based polymers, suitably substantially free of silicone(s).

Suitably, the treatment composition (and suitably therefore any compositions used to prepare the treatment composition) are substantially free from polyurethane. Suitably, the treatment composition (and suitably therefore any compositions used to prepare the treatment composition) are substantially free from polyvinyl alcohol.

Suitably, the treatment composition (and suitably therefore any compositions used to prepare the treatment composition) is substantially free, or contains less than or equal to 1 wt % (suitably less than or equal to 0.1 wt %, suitably less than or equal to 0.01 wt %, suitably less than or equal to 0.001 wt %, suitably less than 0.0001 wt %), of monomers (suitably substantially free of polymerizable monomers) having a molecular weight less than or equal to 10000, suitably having a molecular weight less than or equal to 5000, suitably having a molecular weight less than or equal to 1000, suitably having a molecular weight less than or equal to 500, suitably having a molecular weight less than or equal to 300, suitably having a molecular weight less than or equal to 250.

Suitably, the post-treatment composition is not encapsulated by anything other than the target site or part(s) thereof, suitably not encapsulated within an artificial jacket. Suitably, the post-treatment composition is not contained within an artificial container.

Suitably, the post-treatment composition is substantially not a bolus within the target site, and is suitably distributed within two or more cracks, tears, slits, or fissures at the target site.

Suitably, the post-treatment composition does not fill the target site, but only a part or parts thereof, suitably less than 50 vol. % thereof, suitably less than 20 vol. % thereof.

### Kit of Parts for Forming a Treatment Composition

According to an aspect of the present invention there is provided a kit of parts comprising an activatable composition (suitably as defined herein) and an activator composition (suitably as defined herein). Suitably a treatment composition may be formed by mixing together the activatable composition and the activator composition. The kit of parts may be for use in any of the methods described herein, especially those regarding use of a treatment composition. Suitably both the activatable composition and the activator composition are liquid, suitably liquid solutions or dispersions.

The activatable composition suitably comprises an active precursor component. The activator composition suitably comprises an activator agent. Suitably mixing the activatable composition with the activator composition causes activation of the activatable composition, for instance, by promoting conditions required for the transformation of the active precursor component into the active component.

Suitably the active precursor component of the activatable composition comprises one or more, preferably two or more, activatable moieties. Suitably such activatable moieties may be activated by an activator agent of the activator composition. Suitably the activatable moieties may be activated to react with each other (suitably by a reaction between activatable moieties of two previously-separate molecules of active precursor component).

The activatable composition is suitably substantially free from any activator agents that, by themselves, promote a physical and/or a chemical transformation of the active precursor component. As such, the active precursor component of the activatable composition is suitably physically stable (e.g. remains non-gelled and fluid). The active precursor component is suitably chemically stable (e.g. remains unpolymerized or uncrosslinked).

The activatable composition may comprise one or more deactivator agents (or stabilisation agents), which suitably promote physical and/or chemical stability of the active precursor component within the activatable composition. The deactivator agent(s) may comprise a pH-modifier (e.g. acid, acidifying agent, such as ascorbic acid), suitably to maintain fluidity of the activatable composition. This is particularly useful where the active precursor component is itself pH-sensitive and susceptible to pH-dependent swelling and/or gelling. The deactivator agent(s) may comprise an anti-polymerisation agent, such as an antioxidant, suitably to maintain chemical stability. In a particular embodiment, the deactivator agent(s) comprise both a pH-modifier and an antioxidant, wherein the pH-modifier and the antioxidant may be the same compound (e.g. ascorbic acid). Suitably the activatable composition has a pH between pH 1 and 7, more suitably between 4 and 6.5, more suitably between 5 and 6. Suitably the activatable composition has a pH at or below 7, suitably at or below 6, suitably at or below 5, suitably at or below 4, suitably at or below 3. Suitably the activatable composition has a pH at or above 1, suitably at or above 2, suitably at or above 3. Suitably the activatable composition undergoes swelling and/or gelation at a pH at or above 3, suitably at or above 4, suitably at or above 5, suitably at or above 6. The activatable composition may be buffered. In a particular embodiment the activatable composition has a pH between 5.3-5.7, most suitably a pH of 5.4 or 5.6.

The activatable composition may, however, comprise an activator agent which is substantially inert within the activatable composition, and suitably only participates in activation when mixed with one or more other activator agents, suitably one or more other activator agents present within a complementary activator composition. As such, a complementary pair or complementary group of activator agents (suitably chemical activator agents, for instance, an initiator and an accelerator), which together (i.e. when mixed together in a treatment composition) promote activation of the active precursor component, may initially be separated between activatable and activator compositions, and are suitably only brought together when the treatment composition is formed (which may be during administration). By way of example, the activatable composition may comprise a secondary activator agent (e.g. chemical activator agent, such as an accelerator, e.g. ascorbic acid), and the activator composition may comprise a complementary primary activator agent (e.g. chemical activator agent, such as an initiator, e.g. ammonium persulphate). In this example, when the primary and secondary activator agents are brought together (e.g. upon mixing of the two compositions to form the treatment composition) they form an activating pair which together promote activation of the active precursor component.

The activatable composition may comprise an accelerator, which suitably partners an initiator in the corresponding activator composition. The accelerator may be any suitably accelerator. In a particular embodiment the secondary activator agent is ascorbic acid. As such, the ascorbic acid may perform a dual role of pH-modifier (e.g. acidifying agent) and accelerator.

In an embodiment, the activatable composition comprises a contrast and/or visualisation agent as defined herein.

The activator composition, meanwhile, may suitably comprise one or more activator agents. The activator composition suitably comprises a physical activator agent, most suitably a pH-modifier (e.g. base, alkali, basifying agent, such as sodium hydroxide). Such a physical activator agent is especially useful where the active precursor component of the activatable composition requires a degree of swelling and/or gelation in order for it to be transformed into the active component. The activator composition suitably comprises a chemical activator agent, most suitably a polymerisation initiator, suitably a free-radical initiator (e.g. ammonium persulphate). Such a chemical activator agent is especially useful where the active precursor component of the activatable composition requires a chemical transformation (e.g. polymerisation) in order to transform into the active component. Most suitably the activator composition comprises both a physical activator agent and a chemical activator agent. Suitably the activator composition has a pH between pH 5 and 14, suitably between 7 and 14, suitably between 9 and 14, suitably between 11 and 14. Suitably the activator composition has a pH at or above 5, suitably at or above 6, most suitably at or above 7. Suitably the activator composition has a pH at or below 14, suitably at or below 13, suitably at or below 12. In a particular embodiment the activator composition has a pH between 12 and 14, most suitably about 13. A pH modifier, such as sodium hydroxide, may form a buffered system with ammonium persulphate (and/or with biproducts of ammonium persulphate, such as ammonium sulphate).

The activator composition may comprise a contrast and/or visualisation agent as defined herein. Suitably, if the activatable composition comprises the contrast and/or visualisation agent the activator composition is free of the contrast and/or visualisation agent, and vice versa.

Suitably mixing the activator composition with the activatable composition causes a physical transformation in the active precursor component (e.g. the active precursor component swells and/or gels) and a chemical transformation of the active precursor component, suitably involving polymerisation (or crosslinking) of the active precursor component (suitably free-radical polymerisation) to form the active component. Suitably the chemical transformation does not occur without (and is therefore dependent upon) the physical transformation, though suitably the initial physical transformation is not dependent upon the chemical transformation. In the context of administration of a treatment composition to a candidate subject, the activator composition and the activatable composition may be mixed during the process of administration, and the aforementioned transformation may begin during the administration process, though transformation of the active precursor component into the active component is suitably completed at the target site. As such, the treatment composition suitably retains a degree of fluidity at the target site prior to full transformation. This is especially useful where it is desirable for the treatment composition to flow into and thereby penetrate crevices, tears, cracks, and/or fissures at the target site. It is also preferable to maintain a degree of fluidity to allow the treatment composition to be injected through as small an outlet (e.g. needle) as possible, to thereby minimise damage to the target site during administration.

Suitably the activatable composition comprises the active precursor component, and is suitably free or substantially free of any compounds that cause or promote transformation or degradation of the active precursor component.

In an embodiment:
the activatable composition comprises the active precursor component; and
the activator composition comprises a chemical activator agent (suitably a primary chemical activator agent as defined herein, suitably an initiator, suitably ammonium persulphate) and a physical activator agent (suitably a pH-modifier, suitably a basifier, suitably sodium hydroxide).

In an embodiment:
the activatable composition comprises the active precursor component in a (substantially) non-swollen, non-gelled, or collapsed state; and
the activator composition comprises a physical activator agent (suitably a pH-modifier, suitably a basifier, suitably sodium hydroxide);
wherein mixing the activatable composition and activator composition causes gelling (and suitably swelling of the active precursor component).

In an embodiment:
the activatable composition comprises the active precursor component and one of a pair of chemical activator agents (suitably a secondary chemical activator agent as defined herein, suitably an accelerator, suitably ascorbic acid); and
the activator composition comprises the other of the pair of chemical activator agents (suitably a primary chemical activator agent as defined herein, suitably an initiator, suitably ammonium persulphate).

In an embodiment:
the activatable composition comprises the active precursor component and one of a pair of chemical activator agents (suitably a secondary chemical activator agent as defined herein, suitably an accelerator, suitably ascorbic acid); and
the activator composition comprises the other of the pair of chemical activator agents (suitably a primary chemical activator agent as defined herein, suitably an initiator, suitably ammonium persulphate); and a physical activator agent (suitably a pH-modifier, suitably a basifier, suitably sodium hydroxide).

In an embodiment:
the activatable composition comprises the active precursor component and one of a pair of chemical activator agents (suitably a secondary chemical activator agent as defined herein, suitably an accelerator, suitably ascorbic acid); and a contrast/visualisation agent (e.g. barium sulphate and optionally one or more solubilising or emulsifying components therefor); and
the activator composition comprises the other of the pair of chemical activator agents (suitably a primary chemical activator agent as defined herein, suitably an initiator, suitably ammonium persulphate); a physical activator agent (suitably a pH-modifier, suitably a basifier, suitably sodium hydroxide).

### Active Component and Active Precursor Component

The post-treatment (hydrogel) composition suitably comprises an active component. Suitably the active component is a gel component, suitably a hydrogel component. The treatment composition suitably comprises an active precursor component. Suitably the active precursor component is a precursor to the active component (i.e. of the post-treatment composition). Suitably the active precursor component becomes or is otherwise transformed into the active component following (or during) introduction into a target site. Such a transformation of the active precursor component into the active component may involve a physical change to the component (e.g. swelling and/or gelling). Such a transformation of the active precursor component into the active component may involve a chemical change to the component (e.g. intra- and/or inter-molecular crosslinking). Such a transformation may involve both a physical change and a chemical change. In certain embodiments, the chemical change only occurs following a physical change. In an embodiment, the active component (and/or precursor thereof) is pH-responsive, suitably in that the active component (and/or precursor thereof) undergoes a physical change in response to a change in pH, suitably in that the active component (and/or precursor thereof) undergoes swelling or deswelling in response to a change in pH, suitably in that the active component (and/or precursor thereof) undergoes swelling in response to an increase in pH (suitably when increased from pH 5 to pH 7) or deswelling in response to a decrease in pH (suitably when decreased from pH 7 to pH 5). In an embodiment, the active component (and/or precursor thereof) is temperature-responsive, suitably in that the active component (and/or precursor thereof) undergoes a physical change in response to a change in temperature, suitably in that the active component (and/or precursor thereof) undergoes swelling or deswelling in response to a change in temperature, suitably in that the active component (and/or precursor thereof) undergoes swelling in response to an increase in temperature (suitably when increased from 20° C. to 37° C.) or deswelling in response to a decrease in temperature (suitably when decreased from 37° C. to 20° C.). In an embodiment, the active component (and/or precursor thereof) is both pH- and temperature-responsive.

The physical form (suitably the gel or non-gel form) of the active component is suitably pH-dependent. Suitably the active component exhibits pH-dependent gelling. Suitably the active component exhibits pH-dependent swelling. In the context of the invention, “swelling” and/or “swellability” typically refers to aqueous swelling and/or aqueous swellability (i.e. where a component sequesters water and swells thereby, as per a hydrogel). Most suitably the active ingredient is gelatinous at the pH of the target site. Most suitably the active ingredient is gelatinous within the target site.

The physical form (suitably the gel or non-gel form) of the active precursor component is suitably pH-dependent. Suitably the active precursor component exhibits pH-dependent gelling. Suitably the active precursor component exhibits pH-dependent swelling. Most suitably the active precursor ingredient is gelatinous at the pH of the target site. Most suitably the active precursor ingredient is gelatinous within the target site.

Suitably both the active precursor component and active component are gellable and/or swellable. Suitably both the active precursor component and active component exhibit pH-sensitive swelling and gelling properties.

In some embodiments, the active precursor component and active component are chemically identical (suitably at least in terms of inter- and/or intra-molecular non-ionisable covalent bonds). In some embodiments, the active precursor component and active component are physically identical, though this is usually non-preferred since it is generally preferable for the treatment composition to be substantially mobile in contrast to the post-treatment composition which is comparatively immobile.

Suitably the physical properties (e.g. swellability/gellability) and/or physical form (e.g. gelation state) of the treatment composition and/or post-treatment composition is respectively governed by the active precursor component and/or active component. As such, where the active precursor component and/or active component is pH-sensitive in some respect, suitably the corresponding treatment composition and/or post-treatment composition is also pH-sensitive in the same respect.

Suitably the active component is a synthetic component, suitably a synthetic non-biodegradable component. Suitably the active precursor component is a synthetic component, suitably a synthetic non-biodegradable component. The active component is suitably a hydrogel, more suitably a synthetic hydrogel, most suitably a pH-responsive synthetic hydrogel. The active component (and/or precursor thereof) is suitably a biomimetic hydrogel. The active component (and/or precursor thereof) may be a composite gel, for instance, a composite of two or more gellable materials, potentially a composite of at least one natural and one synthetic gellable material.

Suitably the active precursor component is a polymeric (which in the context of the invention includes copolymeric) compound-such as compound may be termed “active precursor polymer”. Suitably the monomers (which in the context of the invention includes co-monomers as the case may be) of the active precursor polymer are linked together (i.e. polymerised together) via non-hydrolytic bonds (i.e. bonds cleavable by hydrolysis). Suitably the monomers (or co-monomers) of the active precursor polymer are linked together (i.e. polymerised together) via non-enzymatically-cleavable bonds (i.e. suitably not ester-, amide-, glycosidic-, or ether-linkages). Suitably the active precursor polymer comprises (or is derived from) monomers (and/or co-monomers) linked via carbon-carbon bonds (e.g. such as a vinyl polymer)—i.e. a polymer characterised by carbon-carbon polymeric bonds. Suitably the polymer (or copolymer) is derived from vinyl-containing monomers (or co-monomers) and formed by vinyl-polymerisations.

Suitably the active precursor component (which is suitably an active precursor polymer, suitably a microgel) represents a monomer from which the active component is derivable/derived. As such, suitably the active component is formed by polymerising and/or crosslinking a monomeric active precursor component (“active monomer”). Suitably the active component is formed by direct polymerisation (or direct crosslinking) of the active monomer, suitably via polymerizable (or crosslinkable) moieties present within the active monomer (suitably at a surface thereof, especially where the active monomer is itself an active precursor polymer, which in the context of the invention is preferred). Suitably the polymerizable (or crosslinkable) moieties in question may be pre-grafted to the active monomer, for instance, deliberately introduced by a chemical reaction to furnish an active monomer that would otherwise be incapable of polymerisation (or direct polymerisation). In a particular embodiment, the pre-grafted polymerizable (or crosslinkable) moieties are moieties comprising a vinyl group. Suitably the active monomers are polymerizable to form the active component via a free-radical polymerisation reaction, suitably between vinyl groups born by (and suitably pre-grafted to) the active monomers. Suitably the pre-grafted polymerizable (or crosslinkable) moieties are not installed within the active monomer by a free-radical chemical reaction—suitably the pre-grafted moieties are installed using heterolytic chemical reactions, such as condensation reactions. Suitably the active monomers are not polymerized (or crosslinked) via non-pre-grafted moieties (e.g. “spare” vinyl moieties) that may be present within the active monomers (which is suitably a polymer in itself) prior to pre-grafting of the polymerizable (or crosslinkable) moieties. Suitably the active monomer is a polymer, suitably an internally-crosslinked polymer, suitably a polymer particle, most suitably a microgel, and suitably the pre-grafted polymerizable (or crosslinkable) moieties are installed at the surface of the polymer, particle, or microgel. Unless installed at the surface of the active monomer, suitably the relevant moieties could not undergo intermolecular reactions with other active monomers (suitably due to steric and/or electrostatic hindrance). Suitably the active monomers are incapable of undergoing polymerisation unless in a swollen state. As such, suitably polymerisation of the active monomers to produce the active component requires pre-swelling of the active monomers followed by polymerisation (or crosslinking), suitably via free-radical polymerisation (or crosslinking).

Suitably the active component comprises a polymerised array (or network) of active monomers linked together directly, without any intervening cross-linkers (e.g. without additional and distinct crosslinking molecules providing a link between the active monomers).

The active component (and/or precursor) may be any suitable gel or gellable material. Most suitably, the active component (and/or precursor thereof) is or comprises microgel particles, or else any other nanoscopic or microscopic colloidal particles of a crosslinked polymer. The active component precursor (and possibly the active component itself) is suitably injectable, suitably via a syringe needle, suitably wherein the syringe needle corresponds to a Birmingham Gauge between G12 and G 25, suitably between G14 and G23, most suitably between G16 and G21. The active component (and/or precursor thereof) is suitably a biomimetic hydrogel.

The active component (and/or precursor thereof) may suitably be selected from the group consisting of a microgel or cross-linked microgel as defined herein or as set forth in paragraphs [0063] to [00136] and elsewhere in WO 2011/101684 (MANCHESTER UNIVERSITY), proteoglycans, a gellable polysaccharide or polysaccharide-based hydrogel (e.g. cellulose, e.g. a nanofibrillar cellulose NFC hydrogel, hyaluronan/methyl cellulose), a gellable polypeptide or polypeptide-based hydrogel (e.g. gelatin, methacrylated gelatin), a gellable hyaluronate or hyaluronate-based hydrogel, a gellable alginate or aliginate-based hydrogel, a gellable fibrin or fibrin-based hydrogel, a gellable chondroitin or chondroitin-based hydrogel, a gellable polyvinylalcohol (PVA) or PVA-based hydrogel, a gellable polyether or polyether-based hydrogel (e.g. gellable polyalkylene glycol derivatives, e.g. polyethylene glycol, polypropylene glycol derivatives, e.g. PEG tetraacrylate, PEG diacrylate), a gellable polyacrylate or polyacrylate-based hydrogel, a gellable polyalkylacrylate or polyalkylacrylate-based hydrogel, a gellable poly(alkyl)(alk)acrylate or poly(alkyl)(alk)acrylate-based hydrogel (e.g. polymethylmethacrylate, PMMA), a gellable polyacrylamide or polyacrylamide-based hydrogel, a gellable polyalkylacrylamide or polyalkylacrylamide-based hydrogel (e.g. N-isopropylacrylamide NIPAAm), a gellable polyvinylpyrrolidone (PVP) or PVP-based hydrogel, a gellable poly(lactic-co-glycolic acid) or poly(lactic-co-glycolic acid)-based hydrogel (PLGA), chitosan, chitosan hyaluronate, a fibrin sealant-type material, and any combination thereof. The aforementioned gellable materials and hydrogels may be derivatives thereof.

In an embodiment, the active component (and/or precursor thereof) is selected from the group consisting of a microgel or cross-linked microgel as defined herein or as set forth in paragraphs [0063] to [00136] and elsewhere in WO 2011/101684 (MANCHESTER UNIVERSITY), a gellable polyvinylalcohol (PVA) or PVA-based hydrogel, a gellable polyether or polyether-based hydrogel (e.g. gellable polyalkylene glycol derivatives, e.g. polyethylene glycol, polypropylene glycol derivatives, e.g. PEG tetraacrylate, PEG diacrylate), a gellable polyacrylate or polyacrylate-based hydrogel, a gellable polyalkylacrylate or polyalkylacrylate-based hydrogel, a gellable poly(alkyl)(alk)acrylate or poly(alkyl)(alk)acrylate-based hydrogel (e.g. polymethylmethacrylate, PMMA), a gellable polyacrylamide or polyacrylamide-based hydrogel, a gellable polyalkylacrylamide or polyalkylacrylamide-based hydrogel (e.g. N-isopropylacrylamide NIPAAm), a gellable polyvinylpyrrolidone (PVP) or PVP-based hydrogel, a gellable poly(lactic-co-glycolic acid) or poly(lactic-co-glycolic acid)-based hydrogel (PLGA), chitosan, chitosan hyaluronate, a fibrin sealant-type material, and any combination thereof. The aforementioned gellable materials and hydrogels may be derivatives thereof.

Methods of the invention may also be deployed using other compositions known in the art, including implants or implantable compositions (though most suitably the compositions of the invention are not implants) such as swellable (hydrating) implants (e.g. Gelstix™ implants), Nucleofill™ coil implants, PVA and PEG-MMA composite gels, DiscSeal™ implants (from SpineOvations™), PMMA and hyaluronic acid composite gels, Réjuve™, STA-363™, Discogel™, and cellulose-derivatives dissolved in ethanol.

In some embodiments, the active component is simply the gelled counterpart to the active precursor component, yet substantially chemically identical thereto (at least in terms of intra- and/or inter-molecular non-ionisable covalent bonds). As such, any suitable active precursor component which is deliverable to a target site by injection and which forms a stable gel thereafter may be used. However, in preferred embodiments, the active component is formed through a chemical transformation as well as a physical transformation of the active precursor component. This is particularly useful since it facilitates treatment methods which maximise the benefits of initial fluidity (e.g. delivery by injection through a smallest possible outlet to thereby minimise damage to a target site, and/or penetration of small crevices at the target site which may not be possible without sufficient fluidity) and also the final form the active component and post-treatment composition.

Suitably both the active precursor component and the active component are swellable (and suitably both gellable), and thus capable of undergoing swelling and deswelling (suitably gelling and degelling) dependent upon prevailing conditions (e.g. pH or temperature), though most suitably both exhibit a swelling response to a changing pH. Most suitably, the rate of swelling and/or deswelling in response to an identical change in a given parameter or parameters (e.g. pH and/or temperature) is greater for the active precursor component than the active component—i.e. most suitably the active precursor component swells and/or deswells faster than the corresponding active component in response to the same change in conditions. Most suitably, the rate of bulk-gel swelling and/or deswelling in response to an identical change in a given parameter or parameters (e.g. pH and/or temperature) is greater for the treatment composition than the post-treatment composition—i.e. most suitably the bulk gel of the treatment composition swells and/or deswells faster than the corresponding bulk gel of the post-treatment composition in response to the same change in conditions. Suitably the relevant change in conditions is a temperature change. As such, it is preferable that the post-treatment composition is less responsive (at least in terms of bulk swelling rates) to local temperature fluctuations than the parent treatment composition, since then the post-treatment composition is more stable under varying local conditions and less susceptible to problems.

Suitably the active precursor component is particulate, suitably microparticulate. Suitably particles of active precursor component are gellable and/or swellable particles. Suitably a composition comprising said particles of active precursor component is bulk-gellable (i.e. capable of forming a bulk gel or hydrogel, suitably comprising a plurality of gelled particles). Suitably particles of active precursor component are dissolved and/or dispersed in the treatment composition (or activatable composition). Suitably the particles are or comprise polymer(s), suitably a plurality of internally crosslinked polymers (i.e. with crosslinks within a particle rather than between particles). Suitably gellable particles of active precursor component change size (e.g. particle diameter of longest dimension of the particle) in response to a change in pH. Suitably particles of active precursor component increase in size/diameter in response to an increase in pH, and decrease in size in response to a decrease in pH. As such, basification of a composition comprising particles of active precursor component suitably increases particle size and suitably induces gelling of the composition, suitably leading to a bulk gel.

Suitably the active precursor component is or comprises a microgel (or microgel particle).

Suitably the microgel particle constituting the active precursor component is a nanoscopic or microscopic colloidal particle of cross-linked polymer (which includes co-polymer).

In a particular embodiment, the active precursor component is an active precursor polymer defined by the Formula I:

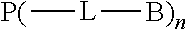

wherein:
P is a polymer;
-L-B is an activatable moiety (suitably a pre-grafted activatable moiety rather than an activatable moiety inherently present within the polymer P), wherein B is an activatable functional group, and L is either a direct bond or a linking group linking an activatable functional group B to the polymer P; and
n is a non-zero integer, suitably an integer greater than or equal to 2.

A plurality of L groups may be independently the same or different to each other. A plurality of B groups may be independently the same or different to each other.

The active precursor polymer of Formula I may be formed by chemically grafting the activatable moiety -L-B to the polymer P, for instance by chemically reacting:
a non-activatable polymer, P′(−Zₚ)ₙ
   with
a reactive activatable compound Zₐ—B
   to produce
active precursor polymer P(-L-B)ₙ
   wherein:
P, -L-B, L, B, and n are as defined herein;
P′ is a part of P connected to Zₚ;
Zₚ is a functionalisable or reactive moiety of the polymer P;
Zₐ is a functionalisable or reactive moiety of the reactive activatable compound;
Zₐ and Zₚ react together so as to link activatable functional group B to the polymer P via L (thus L may be a product of the reaction between Zₐ and Zₚ and/or L or a precursor thereof may form a part of either or both Zₐ and Zₚ).

Zₐ may suitably be defined as Zₐ′-Lₐ-B, wherein Zₐ′ is a reactive group attached to Lₐ, and Lₐ is a part of what becomes L. Zₚ may suitably be defined as Zₚ′-Lₚ, wherein Zₚ′ is a reactive group attached to Lₚ, and Lₚ is a part of what becomes L. Suitably, the product of a reaction between Zₐ and Zₚ is L.

Suitably the non-activatable polymer, P(−Zₚ)ₙ, may comprise more than n Zₚ groups (though suitably no less than n Zₚ groups), but only n Zₚ groups react with reactive activatable compound Zₐ—B to form the active precursor polymer P(-L-B)ₙ.

Respective Z groups (i.e. Zₐ and/or Zₚ) may be the same or different from each other.

The Z groups (i.e. Zₐ and/or Zₚ) may be any suitable reactive group. Suitably the Z groups (i.e. Zₐ and/or Zₚ) are complementary in that they can react together, suitably via a heterolytic coupling reaction (optionally facilitated by an coupling agent), to form a new covalent bond. A skilled chemist would be readily able to select suitable groups. For example, where a non-activatable polymer comprises pendent carboxylic acid groups (suitably as their Zₚ groups), then Zₐ could be or comprise any group that will undergo coupling with the carboxylic acid group(s), for instance, a halogen, hydroxyl, amino or an epoxide group. If a non-activatable polymer comprises pendent amino groups (suitably as their Zₚ groups), then Zₐ could be or comprise any group that will undergo coupling with reacts with the amino group(s), for example, Zₐ could be or comprise —C(O)M, where M is a leaving group, e.g. a halogen such as chloro, or a group that reacts to form a sulfonamide linkage (e.g. Zₐ is or comprises a group such as —S(O)₂Cl).

Zₐ and Zₚ may be coupled following preactivation of either or both Zₐ and Zₚ. For example preactivation may involve preactivation of the carboxylic acid group(s) (e.g. via the formation of an acyl-chloride) or using a coupling agent (e.g. 1-ethyl-3-(3-dimethylaminopropyl) carbodiimide (EDC), carbonyldiimidazole (CDI)). Z may suitably be alkylamino (or a salt thereof), for example ethylamine hydrochloride.

In a particular embodiment, Zₐ is or comprises an epoxide group. In a particular embodiment, Zₚ is or comprises a carboxylic acid group.

L (e.g. as per-L-B) suitably comprises a continuous chain (albeit an optionally substituted or optionally branched chain) of at most 10 atoms (suitably carbon atoms optionally interspersed with one or more heteroatoms) linking B to whichever group or atom L is connected to (e.g. P or Zₐ′), suitably a continuous chain of at most 8 atoms, suitably a continuous chain of at most 6 atoms, suitably a continuous chain of at most 5 atoms. L (e.g. as per-L-B) suitably comprises a continuous chain (albeit an optionally substituted or optionally branched chain) of at least 1 atom linking B to whichever group or atom L is connected to (e.g. P or Zₐ′), suitably a continuous chain of at least 2 atoms, suitably a continuous chain of at least 3 atoms, suitably a continuous chain of at least 4 atoms. In a particular embodiment, L comprises a continuous chain (albeit an optionally substituted or optionally branched chain) of 5 atoms (suitably carbon atoms optionally interspersed with one or more heteroatoms).

In a particular embodiment, L is a direct bond.

In another embodiment, L is a linking group comprising an optionally-substituted and/or optionally-functionalised alkylene chain (e.g. functionalised at either or both termini thereof, though most suitably at the terminus connected to the B group such that the B group is linked to the alkylene chain via the optional functional group), suitably (1-10C)alkylene, suitably (1-7C)alkylene, suitably (1-5C)alkylene, suitably (1-4C)alkylene, suitably (1-3C)alkylene, most suitably (2-3C)alkylene; wherein the alkylene chain is optionally substituted and/or optionally functionalised by one or more functional groups selected from —O—, —C(O)—, —C(O)O—, —OC(O)—, —NRₐ—, —NRₐ—C(O)—, or —C(O)—NRₐ—, wherein Rₐ is H or (1-2C)alkyl or L may be —(OCH₂CH₂)ₙ—, where n is 1 to 50 (inclusive). The alkylene chain may be a short (e.g. 1-4 carbon atom, suitably 2-3 carbon atom) group comprising one or more of these functional groups defined above.

In a particular embodiment, L is a linking group having the formula:

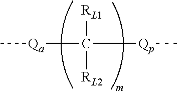

wherein:
R_{L1} and R_{L2} are each independently selected from the group consisting of H and (1-3C)alkyl (most suitably both the same group, most suitably both H);
Qₐ is a group connected to the activatable functional group B, and Qₚ is a group connected to or connectable to the polymer P or part thereof P′; wherein Qₐ and Q_{b} are each independently a direct bond or a divalent moiety selected from the group consisting of —O—, —C(O)—, —C(O)O—, —OC(O)—, —NRₐ—, —NRₐ—C(O)—, or —C(O)—NRₐ—, wherein Rₐ is H or (1-2C)alkyl (most suitably Qₚ is a direct bond, most suitably Qₐ is —C(O)O— or —OC(O)—);
m is a non-zero integer, suitable selected from 1, 2, 3, 4, or 5, more suitably 1, 2, or 3, more suitably 2 or 3, most suitably 3.

In a particular embodiment -L- is:

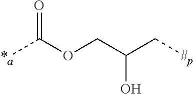

wherein *ₐ is where L connects to B, and #ₚ is where L connects to P. In this example, L L comprises a continuous chain of 5 atoms, and L may be considered a 3 carbon alkylene chain comprising a single —OC(O)— functional group at one terminus thereof.

In a particular embodiment, the activatable (or reactive part) of B is directly connected to L, whether L is a direct bond or linking group. Suitably B groups are capable of undergoing (or activatable to undergo) coupling, suitably heterolytic or homolytic coupling, most suitably free-radical coupling, with one another, suitably in an intermolecular reaction (and B groups are suitably arranged so as preclude their intramolecular reaction). Most suitably B groups provide a means by which the active precursor polymer can mutually crosslink or polymerise together, suitably to form a chain and/or network of interconnected active precursor polymers. As such, the active precursor polymers are suitably active monomers which polymerise to form the active component. Suitably such polymerisation is triggered by a free-radical initiator. Suitably polymerisation is triggered by swelling of the active precursor polymer. Suitably polymerisation only occurs whilst the active precursor polymer is in a swollen state, and then suitably only when triggered by an initiator.

In a particular embodiment, B is or comprises a vinyl group. B may be or comprise any suitable vinyl-containing group. In a particular embodiment, B is or comprises a group —CR₁═CR₂R₃, i.e. the vinyl-containing moiety B has a structure:

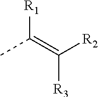

wherein the dotted bond is where the group B forms a bond with L, Zₐ, Zₐ′ or P,
wherein R₁, R₂ and R₃ are suitably selected from H or (1-3C)alkyl.

R₁, R₂ and R₃ are suitably selected from H, methyl or ethyl, especially H or methyl.

In an embodiment of the invention, the reactive activatable compound (Zₐ—B, Zₐ-L-B, Zₐ′-L-B, or Zₐ′-Lₐ-B, as the case may be) is selected from glycidyl methacrylate, glycidylacrylate or other functionalised glycyidylacrylates. Such compounds can be coupled to carboxylic acid, amine or hydroxyl groups within or, more preferably, upon the surface of the polymer P. In a particular embodiment, the reactive activatable compound is glycidyl methacrylate.

The following example reaction scheme illustrates an embodiment of respective Z, L, and B groups:

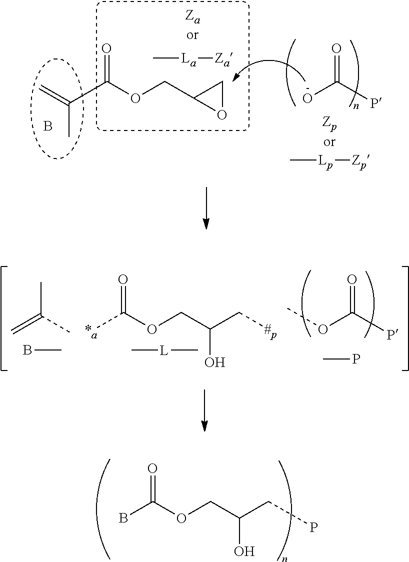

In a particular embodiment, P is a homopolymer or a copolymer, suitably an internally crosslinked homopolymer or copolymer.

Suitably P comprises a hydrophobic co-monomer. Suitably P comprises a physically activatable co-monomer (e.g. a co-monomer which can be “activated” to cause a physical change in P, especially in the bulk properties of P, e.g. swelling/gelling, in response to an environmental change, e.g. temperature and/or pH), which is most suitably a pH-responsive co-monomer. Suitably P comprises both a hydrophobic co-monomer and a physically-activatable co-monomer.

Hence in a particular embodiment, the active precursor polymer comprises copolymeric units defined by the Formula A:

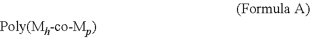

wherein:
   Mₚ is a physically-activatable co-monomer; and
   Mₕ is a hydrophobic co-monomer.

Suitably P comprises a chemically reactive co-monomer (e.g. a co-monomer which can participate in a chemical change, for instance in the formation of new covalent bonds, for instance via polymerisation or crosslinking). Suitably P comprises both a physically activatable co-monomer and a chemically reactive co-monomer, which may include embodiments where such co-monomers are the same and thereby perform a dual function. Most suitably the physically activatable co-monomer and a chemically reactive co-monomer are different co-monomers. Suitably P comprises a hydrophobic co-monomer (Mₕ), a physically activatable co-monomer (Mₚ), and a chemically reactive co-monomer (Mₓ), wherein most suitably each type of co-monomer is different.

Hence in a particular embodiment, the active precursor polymer comprises copolymeric units defined by the Formula A:

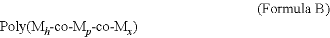

wherein:
   Mₚ is a physically-activatable co-monomer;
   Mₓ is a chemically-reactive co-monomer; and
   Mₕ is a hydrophobic co-monomer.

In a particular embodiment, the active precursor polymer comprises copolymeric units defined by the Formula A1:

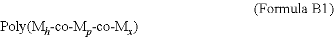

wherein:
   Mₚ is a pH-responsive co-monomer;
   Mₓ is a functional cross-linking co-monomer (which suitably forms internal crosslinks within P); and
   Mₕ is a hydrophobic co-monomer.

Examples of such polymers includes microgel polymers which are described at page 19/line 21 through to page 22/line 8 of WO2007/060424, the relevant contents of which are incorporated herein by reference.

Suitably P is a microgel, suitably a microgel particle.

Suitably, P comprises by weight less than or equal to 95 wt % Mₕ, suitably less than or equal to 85 wt % Mₕ, suitably less than or equal to 75 wt % Mₕ, suitably less than or equal to 70 wt % Mₕ. Suitably P comprises by weight greater than or equal to 30 wt % Mₕ, suitably greater than or equal to 40 wt % Mₕ, suitably greater than or equal to 50 wt % Mₕ, suitably greater than or equal to 60 wt % Mₕ. Most suitably, P comprises by weight between 60 and 70 wt % Mₕ. In a particular embodiment, P comprises 66.8 wt % Mₕ.

Corresponding mol. % (or mol. % ratios) of respective monomers of P, including Mₚ, Mₓ, and/or Mₕ may be calculated from their wt % (or wt % ratios) by reference to the molecular weight of the individual monomers.

Suitably, P comprises, by moles of said monomer compared to the total of all monomers/co-monomers within the polymer P, less than or equal to 95 mol. % Mₕ, suitably less than or equal to 85 mol. % Mₕ, suitably less than or equal to 75 mol. % Mₕ, suitably less than or equal to 70 mol. % Mₕ. Suitably P comprises, by moles of said monomer compared to the total of all monomers/co-monomers within the polymer P, greater than or equal to 30 mol. % Mₕ, suitably greater than or equal to 40 mol. % Mₕ, suitably greater than or equal to 50 mol. % Mₕ, suitably greater than or equal to mol. wt % Mₕ. Most suitably, P comprises, by moles of said monomer compared to the total of all monomers/co-monomers within the polymer P, between 60 and 70 mol. % M. In a particular embodiment, P comprises between 60 and 66 mol % Mₕ, suitably about 63 mol. % Mₕ (e.g. methylmethacrylate-MMA).

Suitably, P comprises by weight less than or equal to 75 wt % Mₚ, suitably less than or equal to 60 wt % Mₚ, suitably less than or equal to 50 wt % Mₚ, suitably less than or equal to 40 wt % Mₚ. Suitably P comprises by weight greater than or equal to 5 wt % Mₚ, suitably greater than or equal to 10 wt % Mₚ, suitably greater than or equal to 20 wt % Mₚ, suitably greater than or equal to 30 wt % Mₚ. Most suitably, P comprises by weight between 30 and 40 wt % Mₚ. In a particular embodiment, P comprises 32.8 wt % Mₚ.

Suitably, P comprises, by moles of said monomer compared to the total of all monomers/co-monomers within the polymer P, less than or equal to 75 mol. % Mₚ, suitably less than or equal to 60 mol. % Mₚ, suitably less than or equal to 50 mol. % Mₚ, suitably less than or equal to 40 mol. % Mₚ. Suitably P comprises, by moles of said monomer compared to the total of all monomers/co-monomers within the polymer P, greater than or equal to 5 mol. % Mₚ, suitably greater than or equal to 10 mol. % Mₚ, suitably greater than or equal to 20 mol. % Mₚ, suitably greater than or equal to 30 mol. % Mₚ. Most suitably, P comprises, by moles of said monomer compared to the total of all monomers/co-monomers within the polymer P, between 30 and 40 mol. % Mₚ. In a particular embodiment, P comprises 33-39 mol. % Mₚ, suitably about 36 mol. % Mₚ.

Suitably, P comprises by weight less than or equal to 5 wt % Mₓ, suitably less than or equal to 4 wt % Mₓ, suitably less than or equal to 3 wt % Mₓ, suitably less than or equal to 2 wt % Mₓ, suitably less than or equal to 1.5 wt % Mₓ. Suitably, P comprises by weight greater than or equal to 0.01 wt % Mₓ, suitably greater than or equal to 0.1 wt % Mₓ, suitably greater than or equal to 0.3 wt % Mₓ, suitably about 0.4 wt %, suitably about 1 wt % Mₓ. Most suitably, P comprises by weight 0.2-0.6 wt % Mₓ, suitably about 0.4 wt %.

Suitably, P comprises, by moles of said monomer compared to the total of all monomers/co-monomers within the polymer P, less than or equal to 5 mol. % Mₓ, suitably less than or equal to 4 mol. % Mₓ, suitably less than or equal to 3 mol. % Mₓ, suitably less than or equal to 2 mol. % Mₓ, suitably less than or equal to 1.5 mol. % Mₓ. Suitably, P comprises, by moles of said monomer compared to the total of all monomers/co-monomers within the polymer P, greater than or equal to 0.01 mol. % Mₓ, suitably greater than or equal to 0.1 mol. % Mₓ, suitably greater than or equal to 0.15 mol. % Mₓ. Suitably, P comprises, by moles of said monomer compared to the total of all monomers/co-monomers within the polymer P, between 0.1 and 0.3 mol. % Mₓ, most suitably about 0.2 mol. % Mₓ.

In a particular embodiment, P comprises by parts by weight (or by wt %):
30 to 95 pbw (wt %) Mₕ; and
5 to 75 pbw (wt %) Mₚ;
wherein, where wt % values are used, the sum of wt % for Mₕ, and Mₚ totals no more than 100 wt %.

In a particular embodiment, P comprises by parts by weight (or by wt %):
60 to 70 pbw (wt %) Mₕ; and
30 to 40 pbw (wt %) Mₚ;
wherein, where wt % values are used, the sum of wt % for Mₕ, and Mₚ totals no more than 100 wt %.

In a particular embodiment, P comprises by parts by weight (or by wt %):
30 to 95 pbw (wt %) Mₕ; and
0.01 and 5 pbw (wt %) Mₓ;
wherein, where wt % values are used, the sum of wt % for Mₕ and Mₓ totals no more than 100 wt %.

In a particular embodiment, P comprises by parts by weight (or by wt %):
60 to 70 pbw (wt %) Mₕ; and
0.2 and 1.5 pbw (wt %) Mₓ;
wherein, where wt % values are used, the sum of wt % for Mₕ and Mₓ totals no more than 100 wt %.

In a particular embodiment, P comprises by parts by weight (or by wt %):
5 to 75 pbw (wt %) Mₚ; and
0.01 and 5 pbw (wt %) Mₓ;
wherein, where wt % values are used, the sum of wt % for Mₚ, and Mₓ totals no more than 100 wt %.

In a particular embodiment, P comprises by parts by weight (or by wt %):
30 to 40 pbw (wt %) Mₚ; and
0.2 and 1.5 pbw (wt %) Mₓ;
wherein, where wt % values are used, the sum of wt % for Mₚ, and Mₓ totals no more than 100 wt %.

In a particular embodiment, P comprises by parts by weight (or by wt %):
30 to 95 pbw (wt %) Mₕ;
5 to 75 pbw (wt %) Mₚ; and
0.01 and 5 pbw (wt %) Mₓ;
wherein, where wt % values are used, the sum of wt % for Mₕ, Mₚ, and Mₓ totals no more than 100 wt %, and suitably totals 100 wt %.

In a particular embodiment, P comprises by parts by weight (or by wt %):
60 to 70 pbw (wt %) Mₕ;
30 to 40 pbw (wt %) Mₚ; and
0.2 and 1.5 pbw (wt %) Mₓ;
wherein, where wt % values are used, the sum of wt % for Mₕ, Mₚ, and Mₓ totals no more than 100 wt %, and suitably totals 100 wt %.

In a particular embodiment, Mₕ is a vinyl-containing monomer, most suitably comprising an optionally substituted acryloyl group or optionally substituted acrylonitrile group, suitably an acryloyl group or (alkyl)acryloyl group (e.g. methacryloyl, ethacryloyl). Suitably Mₕ is an acrylate ester or (alkyl)acrylate ester (e.g. methacrylate ester, ethacrylate ester). Suitably Mₕ is an acrylate alkyl ester or an (alkyl)acrylate alkyl ester (e.g. methyl methacrylate, ethyl acrylate). In a particular embodiment, Mₕ is ethylacrylate (EA). In a particular embodiment, Mₕ is methyl methacrylate.

In a particular embodiment, Mₚ is a vinyl-containing monomer, most suitably comprising an optionally substituted acryloyl group, suitably an acryloyl group or (alkyl)acryloyl group (e.g. methacryloyl, ethacryloyl). Suitably Mₚ is a vinyl-containing monomer comprising a pH-sensitive or ionisable moiety (e.g. an acidic or a basic moiety, e.g. a carboxylic acid group or an amine group), most suitably comprising an optionally substituted acrylic group, suitably an acrylic group or (alkyl)acrylic group (e.g. methacrylic, ethacrylic). Suitably M_{c} is an acrylic acid or an (alkyl)acrylic acid (e.g. methacrylic acid). In a particular embodiment, Mₚ is methacrylic acid (MAA).

In a particular embodiment, Mₓ is a vinyl-containing monomer, most suitably a monomer comprising two or more vinyl groups, suitably a monomer comprising two vinyl groups. Suitably Mₓ is a monomer comprising two or more (most suitably two) substituted acrylonitrile groups and/or optionally substituted acryloyl groups, suitably acryloyl groups and/or (alkyl)acryloyl groups (e.g. methacryloyl, ethacryloyl). Suitably Mₓ is a polyacrylate or poly(alkyl)acrylate, suitably a diacrylate or di(alkyl)acrylate (e.g. dimethacrylate). Suitably Mₓ is a monomer comprising two or more (most suitably two) optionally substituted acryloyl groups, suitably acryloyl groups and/or (alkyl)acryloyl groups, interconnected via a diol, suitably a (2-10C)diol, suitably a (2-4C)diol. Suitably Mₓ is a monomer comprising two or more (most suitably two) acrylate ester or (alkyl)acrylate ester groups (e.g. methacrylate esters, ethacrylate esters), wherein most suitably the acrylate and/or (alkyl)acrylate groups are esters of a common polyol (or diol). In a particular embodiment, Mₓ is ethylene glycol dimethacrylate (EGDMA). In a particular embodiment Mₓ is 1,4-butanediol diacrylate (BDDA).

It will be understood by the skilled person that a polymer may comprise a variety of different Mₕ monomers, a variety of different Mₚ monomers, and/or a variety of different Mₓ monomers, in each case suitably selected from respective monomers as defined herein. Moreover, any polymer may comprise additional monomers not defined herein, though suitably the inclusion of any such monomers should not detract from the functionality and advantages of the invention.

An optionally substituted acryloyl group, in the context of monomers Mₕ, Mₚ, and/or Mₓ, is suitably defined by the formula:

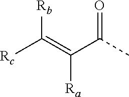

wherein Rₐ, R_{b}, and R_{c} may be the same or different and are each independently an Rₒₚₜ group.

An Rₒₚₜ group is suitably selected from the group consisting of hydrogen, halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (1-6C)alkylamino, (1-6C)dialkylamino, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

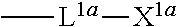

wherein:
   L^{1a} is absent or is selected from O, S, SO, SO₂, N(R₁ₐ), CO, C(O)O, CH(OR₁ₐ), CON(R₁ₐ), N(R₁ₐ)CO, N(R₁ₐ)CON(R₁ₐ), SO₂N(R₁ₐ), N(R₁ₐ)SO₂, OC(R₁ₐ)₂, SC(R₁ₐ)₂ and N(R₁ₐ)C(R₁ₐ)₂, wherein Ria is hydrogen or (1-8C)alkyl; and
   X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein any Rₒₚₜ group is optionally further substituted with one or more Rₒₚₜ as defined above or one or more selected from the group consisting of halogeno or (1-8C)alkyl substituents and/or a substituent selected from hydroxy, mercapto, amino, cyano, carboxy, carbamoyl, ureido, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylureido, N′-(1-6C)alkylureido, N′,N′-di-[(1-6C)alkyl]ureido, N,N′-di-[(1-6C)alkyl]ureido, N,N′,N′-tri-[(1-6C)alkyl]ureido, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

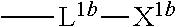

wherein:
   L^{1b} is absent or is selected from O, S, SO, SO₂, N(R_{1b}), CO, C(O)O, CH(OR_{1b}), CON(R_{1b}), N(R_{1b})CO, N(R_{1b})CON(R_{1b}), SO₂N(R_{1b}), N(R_{1b})SO₂, OC(R_{1b})₂, SC(R_{1b})₂ and N(R_{1b})C(R_{1b})₂, wherein R_{1b} is hydrogen or (1-8C)alkyl; and
   X^{1b} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl.

In a particular embodiment, Rₒₚₜ is selected from the group consisting of hydrogen, (1-8C)alkyl, (1-6C)alkoxy, (1-6C)alkylamino, (1-6C)dialkylamino, (2-6C)alkanoyl, (2-6C)alkanoyloxy, (2-6C)alkanoylamino, N-(1-6C)alkyl-(2-6C)alkanoylamino.

Suitably, both R_{b} and R_{c} are hydrogen. Rₐ is suitably hydrogen or (1-4C)alkyl. Suitably both R_{b} and R_{c} are hydrogen whilst Rₐ is suitably hydrogen or (1-2)alkyl.

In a preferred embodiment, P comprises ethylacrylate (i.e. EA, which is the hydrophobic co-monomer, Mh), methacrylic acid (i.e. MAA, which is the pH responsive co-monomer, Mp), and 1,4-butanediol diacrylate (i.e. BDDA, which is the functional cross-linking co-monomer, Mc). Accordingly, a preferred microgel particle P comprises poly(EA/MAA/BDDA).

The poly(EA/MAA/BDDA) used to form the microgel particle P may comprise a maximum mass % EA (hydrophobic monomer) of about 95%, a minimum mass % MAA (pH-responsive monomer) of about 5%, and a minimum mass % BDDA (cross-linking monomer) of about 0.1%. Suitably the mass % of BDDA is within the range of 0.1 to 2%.

In a particular embodiment, the poly(EA/MAA/BDDA) microgel particles comprise about 65.9% EA, about 33.1% MAA and about 1.0% BDDA based on the total monomer mass. This may be defined as a mass ratio of EA/MAA/BDDA as 65.9/33.1/1.0, or as a mole ratio of EA/MAA/BDDA is 130.4/76.0/1.0.

In another preferred embodiment, the microgel particle comprises methylmethacrylate (i.e., MMA, which is the hydrophobic co-monomer, Mh), methacrylic acid (i.e., MAA, which is the pH-responsive co-monomer, Mp) and ethyleneglycol dimethacrylate (i.e., EGDMA, which is the functional cross-linking co-monomer, Mc). Accordingly another preferred microgel particle comprises poly(MMA/MAA/EGDMA).

The poly(MMA/MAA/EGDMA) used to form the microgel particle may comprise a maximum mass % MMA (hydrophobic monomer) of about 95%, a minimum mass % MAA (pH-responsive monomer) of about 5%, and a minimum mass % EGDMA (cross-linking monomer) of about 0.1%. Suitably the mass % of EGDMA is within the range of 0.1 to 2%.

In a particular embodiment, the poly(MMA/MAA/EGDMA) of the microgel particles comprises about 60-70% MMA, about 30-40% MAA and 0.1 to 1% EGDMA based on the total monomer mass.

In a particular embodiment, the poly(MMA/MAA/EGDMA) of the microgel particles comprises about 66.8% MMA, about 32.8% MAA and about 0.4% EGDMA based on the total monomer mass. This may be defined as a mass ratio of MMA/MAA/EGDMA of 167/82/1.0, or as a mole ratio of MMA/MAA/EGDMA is 320/185/1.0.

Suitably, the active precursor polymer, P(-L-B)ₙ, is formed by grafting n-L-B groups to physically activatable monomers, Mₚ, present within the polymer P, suitably to pH-responsive monomers, suitably to carboxylic acid groups thereof. Suitably, however, at least some (suitably at least 20%, suitably at least 50%, suitably at least 70%) of the physically activatable monomers remain unreacted (or unfunctionalized) following grafting, and suitably the active precursor polymer retains pH-dependent swelling properties.

Suitably the concentration of monomers of the active precursor polymer functionalised with an -L-B group (or having an -L-B group grafted thereto) is between 0.1 and 60 mol. % with respect to all of the co-monomers present in the active precursor polymer, suitably between 0.5 and 30 mol. %, suitably between 1 and 20 mol. %, suitably between 1.5 and 15 mol. %, suitably between 1.7 and 10 mol. %, suitably between 2 and 8 mol. %, suitably between 3 and 7 mol. %, suitably between 4 and 6 mol. %. Such concentrations may be determined by establishing how many of a known concentration of monomers with reactive groups have undergone a grafting reaction, for instance by titrating to establish before and after concentrations of free carboxylic acid groups, and extrapolating the result to a mol. % of all monomers (i.e. diluted by known concentrations of monomers without reactive groups—unreactive monomers). Such titration methodologies are outlined in WO2011/101684 (University of Manchester), for instance as per detailed in Methods 4 and 4a respectively at paragraphs [00192] and [00193] of WO2011/101684.

### Calculation

The concentration (mol. %) of monomers of the active precursor polymer which are functionalised with an -L-B group (or having an -L-B group grafted thereto) may be calculated via the following protocol.

The pre-functionalised active precursor polymer may be considered to consist of reactive monomers (i.e. monomers which can react with a compound to graft-L-B groups thereto) and unreactive monomers, and the sum of the mol. % concentrations of reactive monomers and unreactive monomers in the pre-functionalised active precursor polymer is 100 mol. %, as per the equation:

$${M}_{reactive} + {M}_{unreactive} = 100$$

where M_{reactive} is the mol. % of reactive monomers in the pre-functionalised active precursor polymer, and M_{unreactive} is the mol. % of unreactive monomers (which may include crosslinking monomers) in the same polymer. As such, mol. % functionalisation or a mol. % concentration of functionalised monomers of the active precursor polymer (i.e. monomers, whether reactive or unreactive, functionalised with an -L-B group or having an -L-B group grafted thereto) may be calculated by the equation:

$${M}_{functionalised} = {M}_{reactive} - {M}_{residual}$$

$${M}_{functionalised} + {M}_{residual} + {M}_{unreactive} = 100$$

where M_{functionalised} is mol. % of functionalised monomers (i.e. the reactive monomers that have reacted and become functionalised), M_{reactive} is the initial mol. % of reactive monomers in the pre-functionalised active precursor polymer, and M_{residual} is the residual mol. % of reactive monomers in the post-functionalised active precursor polymer (i.e. those reactive monomers which are left unreacted).

The molar ratio of reactive monomers to unreactive monomers in any given pre-functionalised active precursor polymer is suitably derivable from (and suitably equates to) the relative molar quantities of input monomers used in the polymerisation reaction to form the pre-functionalised active precursor polymer. As such, this ratio (r) is known or predetermined and gives rise to the equations:

$$r = \frac{{M}_{reactive}}{{M}_{unreactive}} = \frac{{M}_{reactive}}{100 - {M}_{reactive}} = \frac{100 - {M}_{unreactive}}{{M}_{unreactive}} = \frac{{M}_{functionalised} + {M}_{residual}}{{M}_{unreactive}}$$

$${M}_{unreactive} = \frac{{M}_{reactive}}{r}$$

where r is the ratio of reactive monomers to unreactive monomers in any given pre-functionalised active precursor polymer or r is the ratio of functionalised+residual reactive monomers to unreactive monomers in any given post-functionalised active precursor polymer.

The same ratio holds for absolute molar concentrations, for instance, where the absolute molar concentration of reactive monomers in a solution of a pre-functionalised active precursor polymer is experimentally determined, for instance, by titration (especially where the reactive groups are pH-active and have ionisable groups):

$$r = \frac{[ {M}_{reactive} ]}{[ {M}_{unreactive} ]} = \frac{[ {M}_{functionalised} ] + [ {M}_{residual} ]}{[ {M}_{unreactive} ]}$$

where r is the ratio of reactive monomers to unreactive monomers in any given pre-functionalised active precursor polymer or r is the ratio of functionalised+residual reactive monomers to unreactive monomers in any given post-functionalised active precursor polymer; [M_{reactive}] is an absolute molar concentration of reactive monomers (present within the active precursor polymer optionally alongside unreactive monomers) in a solution or dispersion of a particular amount or concentration (e.g. expressed as wt %) of pre-functionalised active precursor polymer; [M_{runctionalised}] is an absolute molar concentration of functionalised monomers (present within the post-functionalised active precursor polymer alongside residual reactive monomers and optionally alongside unreactive monomers) in a solution or dispersion of a particular amount or concentration (e.g. expressed as wt %) of post-functionalised active precursor polymer; [M_{residual}] is an absolute molar concentration of residual reactive monomers (present within the post-functionalised active precursor polymer alongside functionalised monomers and optionally alongside unreactive monomers) in a solution or dispersion of a particular amount or concentration (e.g. expressed as wt %) of post-functionalised active precursor polymer; and [M_{uneactive}] is an absolute molar concentration of unreactive monomers (present within the active precursor polymer optionally alongside unreactive monomers) in a solution or dispersion of a particular amount or concentration (e.g. expressed as wt %) of pre-functionalised active precursor polymer.

Invariably, a titration or other such analysis is performed on a solution or dispersion of a known or predetermined concentration of the relevant polymer (potentially determined as a wt % of said polymer). As such, the same titration (or other such analysis) may be performed on solutions or dispersions of both pre-functionalised and post-functionalised polymers to respectively deduce, after adjusting for any differences in overall polymer concentration between respective pre-functionalised and post-functionalised samples (which can be normalised by using a relevant multiplier), initial and residual reactive monomer concentrations (before and after functionalisation) to thereby provide a mol. % concentration of functionalised monomers of the active precursor polymer. As such, mol. % concentration of functionalised monomers of the active precursor polymer (i.e. monomers, whether reactive or unreactive, functionalised with an -L-B group or having an -L-B group grafted thereto) may be calculated by the equations:

$$mol . \% functionalisation , {M}_{functionalised} = \frac{[ {M}_{reactive} ] - [ {M}_{residual} ]}{[ {M}_{reactive} ] + [ {M}_{unreactive} ]} \times 100$$

$${M}_{functionalised} = \frac{[ {M}_{reactive} ] - [ {M}_{residual} ]}{[ {M}_{reactive} ]} \times \frac{r}{r + 1} \times 100$$

where concentrations [M_{reactive}], [M_{residual}], and [M_{unreactive}] are normalised concentrations of the respective monomers based on the same polymer concentration for both pre- and post-functionalised polymer samples. For instance, if titrations are performed on solutions/dispersions of each of the pre-functionalised polymer and post-functionalised polymer, but the post-functionalised polymer is present at half the concentration of the pre-functionalised polymer in its corresponding titration, the unnormalized [M_{residual}] value obtained from the titration of the post-functionalised polymer would be transformed into a normalised [M_{residual}] value by multiplying by 2. The skilled person will also appreciate that other methods may be utilised to determine the degree of functionalisation.

As aforementioned, the active precursor polymer (and thus active monomers) may be a gellable particle, such as a microgel particle. Examples of suitable microgel particles serving as a pre-grafted polymer P in the context of the present invention are described in WO2007/060424 (University of Manchester), the entire contents of which are incorporated herein by reference. In particular, WO2007/060424 describes various pH-responsive microgel particles. Suitably, the active precursor component may be or comprise microgels of WO2007/060424 which have been modified (or functionalised) by pre-grafting onto the surface thereof polymerizable (or crosslinkable) moieties, such as vinyl groups. Such modifications are described in WO2011/101684 (University of Manchester), the entire contents of which are also incorporated herein by reference. Thus any of the vinyl-grafted microgel particles described in WO2011/101684 may suitably serve as active precursor polymers of the invention, and the skilled person will readily appreciate how such vinyl-grafted microgel particles map to the formula P-L-B as defined herein.

The active precursor component suitably is or comprises one or more microgel particles which undergo a conformational change in response to an environmental change (e.g. pH and/or temperature), suitably between a “non-swollen” and a “swollen” state. The active precursor component suitably is or comprises one or more pH-responsive microgel particles, suitably microgel particles which undergo a conformational change in response to a variation in pH, suitably between a “non-swollen” and a “swollen” state.

The diameter (or largest dimension) of a microgel particle suitable depends on their water content, which in turn suitably depends on the prevailing environment (e.g. pH and/or temperature, most suitably pH). Suitably the microgel particles respond to a pH change through a corresponding change in protonation (or ionisation) state of certain moieties therein (e.g. protonation or deprotonation of pendant carboxylic acids), and water content generally increases as ionisation levels increase. Suitably the microgel particles exhibit a non-swollen state prior to administration (or within an activatable composition), and a swollen state (or partially-swollen state) after administration. Some swelling may occur during administration.

In a non-swollen state, the average diameter (or average largest dimension) of the microgel particle is suitably less than or equal to 100 □m, suitably less than or equal to 50 □m, suitably less than or equal to 20 □m, suitably less than or equal to 10 □m, suitably less than or equal to 5 □m, suitably less than or equal to 1 □m, suitably less than or equal to 10 □m. More suitably, in a non-swollen state, the average diameter (or average largest dimension) of the microgel particle is between 1 and 1000 nm, suitably between 10 and 750 nm, suitably between 20 and 500 nm, most suitably between 50 and 100 nm. Suitably microgel particles of a treatment composition or activatable composition suitably exhibit any one of the aforementioned sizes (i.e. pre-administration).

As the swelling of said microgel particles is suitably caused by a flow of water into the particle, when the particle is in a swollen state the microgel particle suitably comprises at least about 50% (w/w) water, suitably at least about 70% (w/w) water, more suitably at least about 85% (w/w) water, suitably at least about 90% (w/w) water, or suitably at least about 95% (w/w) water. Suitably the microgel particle comprises less water at low pH than at a relatively higher pH. Suitably, microgel particles of a post-treatment composition exhibit any one of the aforementioned water-contents.

Suitably, in a fully swollen state, the average diameter (or average largest dimension) of the microgel particle (especially where the microgel particle is a precursor microgel particle) is at least 50% greater than in the corresponding non-swollen state, more suitably at least 75% greater, more suitably at least 100% greater, and potentially at least 200% greater. Suitably, in a fully swollen state, the average diameter (or average largest dimension) of the microgel particle (especially where the microgel particle is a precursor microgel particle) is at most 1000% greater than in the corresponding non-swollen state, suitably at most 800% greater, suitably at most 500% greater, suitably at most 400% greater, suitably at most 300% greater. Suitably, the particle size of microgel particles of a treatment composition or activatable composition exhibit an increase by any one of the aforementioned factors within the post-treatment composition.

Suitably, in a collapsed state (e.g. in an aqueous medium at pH 5 at SATP), the microgel particles have a particle size between 10-200 nm, more suitably between 20-150 nm, more suitably between 50-100 nm, most suitably 60-90 nm. Suitably, in a collapsed state (e.g. in an aqueous medium at pH 5 at SATP), the microgel particles have an average (mean) particle size between 10-200 nm, more suitably between 20-150 nm, more suitably between 50-100 nm, most suitably 60-90 nm.

The microgel particles of an active precursor component are suitably derived from monomers (or co-monomers) at least some of which bear ionisable groups, suitably which are capable of changing protonation state, suitably groups which are substantially unionised at a pH at or below 2, suitably below 1, but are substantially ionised at a pH above 10. Suitably said ionisable groups are acid groups and thus at least some of the monomers are acidic monomers. Suitably such acid groups may be carboxylic acid groups. Suitably the pKₐ of the acid group(s) is at or below 6, suitably at or below 5, suitably at or below 4.5, suitably at or below 4. Suitably the pKₐ of the acid group(s) is at or above 1, suitably at or above 2, suitably at or above 3. Suitably protonation and/or deprotonation of said ionisable groups affects the swelling state of the microgels, and hence the bulk gelation state of the composition containing said microgels.

Most suitably the microgel particles (and suitable a composition comprising said microgels) are in a swollen state (or is in a gel state) at the pH prevailing at the target site. Suitably the microgel particles are in a swollen state at physiological pHs, suitably at a pH between 5.5 and 8, suitably between pH 6 and pH 8, suitably between pH 6 and pH 7.5.

It will be understood that a composition comprising said microgel particles will exhibit bulk-gelling properties corresponding to the aforementioned changes in the microgel particles themselves.

### Additional Components

The treatment composition may suitably comprise one or more activator agents. Said activator agent(s) suitably promote transformation of the active precursor component(s) into the active component(s), be it physical transformation, chemical transformation, or a combination thereof. As such, the post-treatment composition may suitably comprise one or more activator agents, or one or more products derived therefrom (e.g. by-products of the relevant transformation process).

An activator agent may be a physical activator agent, which suitably promotes a physical transformation of the active precursor component(s). The physical activator agent is suitably a swelling- or gelling-inducing agent, which suitably causes the active precursor component(s) to swell and/or gel. Suitably the physical activator agent is or comprises a pH modifier, which suitably causes a pH change that promotes the aforementioned physical transformation(s), most suitably swelling and/or gelling. Suitably the pH modifier is a basifying agent, which suitably causes a pH increase that promotes the aforementioned physical transformation(s). Most suitably the basifying agent is an inorganic base, most suitably an inorganic base that produces hydroxide ions when mixed with (or dissolved in) water, most suitably an inorganic oxide or inorganic hydroxide, suitably a metal oxide or metal hydroxide, suitably a metal (I) or metal (II) oxide or hydroxide, suitably an alkali or alkaline earth metal oxide or hydroxide, suitably an alkali metal oxide or hydroxide, suitably sodium hydroxide.

An activator agent may be a chemical activator agent, which suitably promotes a chemical transformation of the active precursor component(s). The chemical activator agent suitably promotes the formation of new covalent bonds, suitably new intermolecular covalent bonds, suitably between active precursor component molecules. The chemical activator agent suitably activates one or more activatable moieties and/or activatable functional groups within the active precursor component, suitably to promote reactivity with other moieties or groups within the activator precursor component, suitably to promote free-radical reactivity with other moieties or groups within the activator precursor components (which may themselves also be activatable moieties and/or activatable functional groups, which may be activated as part of a free-radical chain reaction). Suitably the chemical activator agent promotes polymerisation and/or crosslinking between active precursor components, suitably free-radical polymerisation and/or crosslinking between active precursor components, suitably directed polymerisation and/or crosslinking between active precursor components (i.e. suitably without any intervening cross-linking moieties or compounds). Suitably the chemical activator agent is an oxidising agent, a reducing agent, and/or a free radical initiator. Suitably the chemical activator agent is an initiator, suitably a free-radical initiator, suitably an initiator which promotes crosslinking or polymerisation between vinyl groups. Suitably the activator agents comprise at least two chemical activator agents, one of which is a primary chemical activator agent, and another which is a secondary chemical activator agent, wherein suitably the secondary chemical activator agent promotes or accelerates the chemical activating capabilities of the primary chemical activator agent. For instance, the primary chemical activator agent may be an initiator, whilst the second chemical activator agent may be an accelerator, and suitably the accelerator accelerates the initiation provided by the initiator. The chemical activator agent(s) are suitably water-soluble.

Suitable water-soluble initiators include:

### Anionic Initiators:

initiators of the general formula [M]S₂O₈²⁻, wherein M is a cation such as K⁺, Na⁺ or NH₄⁺, or a divalent cation. Ammonium persulfate, (NH₄⁺)₂S₂O₈²⁻, is a specific example.
an organic anionic azo initiator of formula:

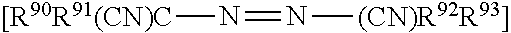

wherein:
R⁹⁰ and R⁹² may be independently selected from a group consisting of H; CH₃; a linear or branched (1-10C)alkyl group; or a —NH-(1-10C)alkyl or —N[(1-10C)alkyl]₂ group; and
R⁹¹ and R⁹³ may be CR⁹⁴COOH (wherein R⁹⁴ may be —CH₂—, —CH₂CH₂— or a linear, or branched (1-20C)alkylene chain) or phenyl which is optionally substituted (for example, by one to three substituent groups selected from halo, (1-6C)alkyl, amido, amino, hydroxy, nitro, and (1-6C)alkoxy). A particularly suitable initiator belonging to this group is azobiscyanopentanoic acid (also known as 4,4′-azobis(4-cyanovaleric acid)).

### Cationic Initiators:

a cationic amine initiator of structural formula:

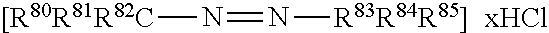

wherein R⁸⁰, R⁸¹, R⁸³ and R⁸⁴ may be independently selected from a group consisting of H; CH₃; a linear or branched (1-10C)alkyl group; a —NH-(1-10C)alkyl or —N[(1-10C)alkyl]₂ group; and wherein R⁸² and R⁸⁵ may be C(═NR⁸⁶)NH₂ wherein R⁸⁶ may be independently selected from a group consisting of H; CH₃; a linear or branched (1-10C)alkyl group. For example, a specific example is propanimidamide, 2,2′-azobis [2-methyl-, dihydrochloride]. This initiator is also known as V50.

### Peroxide Initiators:

a peroxide initiator defined by the structural formula:

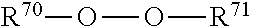

wherein R⁷⁰ or R⁷¹ may be independently selected from a group consisting of H; CH₃; a linear or branched (1-10C)alkyl group; a —NH-(1-10C)alkyl or —N[(1-10C)alkyl]₂ group; or phenyl which is optionally substituted (for example, by one to three substituent groups selected from halo, (1-6C)alkyl, amido, amino, hydroxy, nitro, and (1-6C)alkoxy). Suitable water soluble ultraviolet photoinitiators are of the formula:

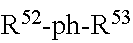

where R⁵² is HO—(CH₂)₂— and R⁵³ is —C(O)C(OH)(CH₃)₂ and ph represents a phenyl ring. A particular initiator according to this formula is known as Irgacure 2959.

Most suitably the initiator is ammonium persulphate.

Where an accelerator is used, suitably as a secondary chemical activator agent, said accelerator is suitably water-soluble. Suitable examples of such accelerators include TEMED (1,2-bis(dimethylamino)ethane, N,N,N′,N′-Tetramethylethylenediamine) and ascorbic acid (also known as DL-ascorbic acid).

Most suitably, the primary chemical activator agent is ammonium persulphate and the secondary chemical activator agent is ascorbic acid.

The treatment composition may suitably comprise a contrast or visualisation agent. The contrast or visualisation agent may suitably facilitate administration of the treatment composition to a target site (e.g. where image-guided administration is involved). For instance, a visualisation agent such as barium sulphate can facilitate visualisation by C-arm fluoroscopy and/or X-rays during a discography procedure. As such, the visualization agent is suitably visualizable by fluoroscopy and/or by X-ray imaging. The contrast or visualisation agent may assist monitoring of the fate of the post-treatment composition following administration. As such, the post-treatment composition may suitably comprise the contrast or visualisation agent, or a product derived therefrom. In a particular embodiment, the contrast/visualisation agent is barium sulphate.

Where a contrast or visualisation agent is used, it may be preferably to incorporate additional solubilising or emulsifying components to facilitate solubility or emulsification of the contrast/visualisation agent, especially for instance where the contrast/visualisation agent has limited solubility in water. Such additional solubilising or emulsifying compounds may be selected from the group consisting of a salt of a multidentate anion (e.g. citrate salt, e.g. sodium citrate), a polyol (e.g. a sugar, e.g. sorbitol, e.g. D-sorbitol), an antifoaming agent (e.g. simethicone), a hydrophilic polymer (e.g. a polyalkoxylene glycol polymer, e.g. a PEG, e.g. PEG 400), or any combination thereof. In a particular embodiment, the contrast/visualisation agent is barium sulphate, and the treatment composition comprises additional solubilising or emulsifying agents including a citrate salt, a sugar, a silicon-based antifoaming agent, and a hydrophilic polymer.

The treatment composition and/or post-treatment composition may suitably comprise one or more further bioactive material(s). In an embodiment, a bioactive materials composition comprises one or more of the one or more bioactive material(s). Said bioactive materials composition may be mixed with one or more other compositions to form the treatment composition (e.g. in a syringe, such as a multi-barreled syringe). As such, any kit of parts described herein may additionally comprise a bioactive materials composition, or one or more thereof depending on the number of bioactive materials and the need to keep them separate. In an embodiment, the bioactive material(s) is or comprises a pharmaceutical or biopharmaceutical, more suitably a biopharmaceutical. The biopharmaceutical may be selected from the group consisting of: a biological product extracted from a living system (e.g. whole blood or blood components, stem cells, living cells, antibodies, hormones), a biologically-active produced derived by recombinant DNA (e.g. hormones, monoclonal antibodies, fusion proteins, blood factors, growth factors, interferons, interleukins, and other proteins), vaccines, gene-therapy products (e.g. viruses containing genetic material), and any combination thereof. In an embodiment, the bioactive materials is or comprises one or more cells, suitably cells of the target location for treatment compositions of the invention. For example, the bioactive materials may be or comprise one or more nucleus pulposus cells. The cells are suitably mammalian cells. Examples of suitable cells, which may be included in compositions of the invention, include chondrocytes (e.g. autologous or autogenous). Examples of suitable stem cells, which may be added to the composition include mesenchymal, haematopoietic etc., including embryonic and cloned stem cells. In addition, the composition administered may further comprise collagen and/or proteoglycans. It will be expected that adding nucleus pulposus cells to the composition will increase the rate of recovery of the subject. In an embodiment, the bioactive materials(s) comprise a mixture of living cells, suitably NP cells and/or stem cells (e.g. MSCs, e.g. hypoxid-cultured MSCs designed to survive in the IVD environment).

Suitably, the treatment composition and/or post-treatment composition is free of any, some, or all of the aforesaid further bioactive material(s).

It will be understood that aforementioned components of the treatment composition may initially be split between two or more separate compositions, suitably between an activatable composition and an activator composition, which are eventually mixed to form the treatment composition. Such embodiments are discussed further in relation to a kit of parts.

Transforming Active Precursor Component into Active Component and Treatment Composition into Post-treatment Composition

Treatments according to the invention suitably involve a transformation of the treatment composition into a post-treatment composition. Suitably such a transformation comprises a physical transformation, most suitably swelling and/or gelling, suitably to form a hydrogel. Suitably such a transformation comprises a chemical transformation, suitably involving the formation of new (suitably non-ionisable) covalent bonds, most suitably involving polymerization or cross-linking, suitably involving polymerization or cross-linking of an active precursor component suitably to form an active component. Most suitably, such a transformation involves both the aforesaid physical transformation and chemical transformation. Most suitably a chemical transformation occurs after an initial physical transformation, though physical transformation(s) may continue following a chemical transformation. Suitably a treatment composition (or a component thereof) undergoes swelling, suitably involving swelling of an active precursor component (and/or particles thereof, e.g. microgel particles thereof), prior to chemical transformation. Suitably the chemical transformation is impossible without pre-swelling of the treatment composition and/or active precursor component. Suitably physical transformation increases the reactivity or coupling potential of activatable moieties of the active precursor component, and thereby facilitates or kinetically-favours a chemical transformation.

In a particular embodiment, the treatment composition (or activatable composition) comprises gellable particles, suitably gellable polymeric particles, suitably microgel particles. Such gellable particles are suitably capable of undergoing a physical transformation, suitably swelling and/or gelling. The gellable particles may swell to afford a treatment composition or a post-treatment composition as a bulk gel, such as a hydrogel.

In some embodiments, the gellable particles comprise reactive moieties (e.g. activatable moieties). Gellable particles with reactive moieties may suitably swell (suitably to afford a treatment composition as a bulk gel) before said reactive moieties partake in a chemical transformation as described herein. Most suitably, a post-treatment composition formed from a chemical transformation (e.g. polymerization of an active precursor component to form an active component) and suitably also a physical transformation (e.g. swelling/gelling) is less fluid, more viscous, and/or harder (e.g. with a higher young's modulus) than a corresponding treatment composition. This suitably enables a treatment composition (albeit potentially partially-transformed) to fill slits, tears, and/or crevices at a target site before complete hardening or curing, suitably to thereby precipitate advantageous effects of the invention.

The swelling ratio (q=V/V_{coll}) defines the degree of swelling of the gellable particles (e.g. microgel particles). V is the gellable particle volume measured in a partially swollen or fully swollen configuration. V_{coll} is the volume of the non-swollen, collapsed configuration of the gellable particles. The value for q during chemical transformation (e.g. vinyl-coupling reaction) is suitably 1.1-500. Preferably, the value for q is between 3-100.

Where the active precursor polymer is a microgel particle bearing vinyl-containing moieties grafted onto the surface thereof, suitably these microgel particles may undergo a free-radical coupling reaction directly with the vinyl-containing moieties grafted onto the surfaces of adjacent microgel particles to form a direct covalent bond therebetween. The first step of the chemical transformation suitably involves providing microgel particles that have vinyl-containing moieties grafted on to their surfaces. The next step suitably involves bringing the surfaces of the adjacent particles into contact with one another, suitably by causing adjacent microgel particles to swell by appropriately varying temperature or pH (as described herein), suitably by increasing the pH. The swelling of the microgel particles as they hydrate suitably causes the surfaces of adjacent particles to contact one another and even overlap to form interpenetrating regions of gelled polymer. This suitably disposes the surface grafted vinyl-containing moieties of adjacent microparticles in close proximity to one another to facilitate the free-radical coupling of the vinyl moieties, as discussed further below.

The reaction between the vinyl-containing moieties grafted onto the surface of adjacent microgel particles is achievable by free-radical chemistry using techniques well known in the art. Such a reaction must suitably take place in an aqueous medium, so suitably water-soluble reactants (e.g. activator agents) should be used. Suitably any reactants possess little or no toxicity to the candidate subject.

Suitably, the chemical transformation reaction is conducted in the presence of a free radical initiator (hereinafter referred to as an initiator), which is suitably a water-soluble initiator. Suitably, the initiator is responsive to pH, temperature and/or ultraviolet radiation.

In a particular embodiment, the activator agents comprise a chemical activator agent such as an initiator, such as ammonium persulphate. In a particular embodiment, the activator agents comprise a physical activator agent such as a pH-modifier, suitably a basifying agent, such as sodium hydroxide.

The free-radical coupling reaction may also be conducted in the presence of a suitable water soluble accelerator (which is suitably also a chemical activator agent, suitably for use in conjunction with an initiator). Suitable examples of such accelerators include TEMED (1,2-bis(dimethylamino)ethane, N,N,N′,N′-Tetramethylethylenediamine) and ascorbic acid (also known as DL-ascorbic acid).

A skilled chemist will be able to select appropriate experimental conditions in order to carry out the vinyl coupling reactions.

Suitably the microgel particles swell at body temperature (e.g. 37° C.) and/or the pH of the target site.

Suitably chemical transformation (e.g. cross-linking of the microgel particles) proceeds at normal body temperature.

### Properties of the Bound Microgel Particle Compositions of the Invention

Post-treatment compositions of the invention may suitably belong to the class of materials known as hydrogels. Suitably, however, they differ from conventional hydrogels because they are composed of bound or linked microgel particles.

The elastic modulus (G′) of post-treatment compositions of the invention will be dependent on the method used for their preparation. Suitably the values for G′, as measured by dynamic rheology, will typically be greater than 10 Pa.

The swelling characteristics of post-treatment compositions of the invention can again be defined by the swelling ratio (as defined hereinbefore). The value for q will typically be between 1.2 and 500. For the specific application of inter-vertebral disc repair, the swelling ratio is preferred to be between 3 and 200.

The post-treatment compositions of the present invention suitably have significant critical strain values (□*). The critical strain value is the value for the strain, measured by a rheometer, at which the elastic modulus (G′) first reaches a value of 95% of that measured when □=1.0%. The preferred range for □* for the compositions of the invention is 2 to 500%, more preferred is 5 to 300%, and even more preferred is 5 to 200%.

The post-treatment compositions of the invention are suitably non-porous. Suitably, post-treatment compositions of the invention provide insufficient space or pores to load agents.

Suitably the post-treatment compositions of the invention are pH-responsive. Suitably post-treatment compositions of the invention are substantially non-fluid and substantially immobile a pHs prevailing within the target site.

### Particular Embodiments of Treatment Compositions

In a particular embodiment, the treatment composition comprises:
an active precursor component (e.g. active precursor polymer); and
a physical activator agent (e.g. pH-modifier, e.g. a base, e.g. NaOH).

In a particular embodiment, the treatment composition comprises:
an active precursor component (e.g. active precursor polymer); and
a contrast agent and/or visualisation agent (e.g. BaSO₄).

In a particular embodiment, the treatment composition comprises:
an active precursor component (e.g. active precursor polymer);
a physical activator agent (e.g. pH-modifier, e.g. a base, e.g. NaOH); and
a contrast agent and/or visualisation agent (e.g. BaSO₄).

In a particular embodiment, the treatment composition comprises:
an active precursor component (e.g. active precursor polymer); and
a chemical activator agent (e.g. an initiator).

In a particular embodiment, the treatment composition comprises:
an active precursor component (e.g. active precursor polymer);
a chemical activator agent (e.g. an initiator); and
an accelerator (e.g. ascorbic acid).

In a particular embodiment, the treatment composition comprises:
an active precursor component (e.g. active precursor polymer);
a physical activator agent (e.g. pH-modifier, e.g. a base, e.g. NaOH); and
a chemical activator agent (e.g. an initiator).

In a particular embodiment, the treatment composition comprises:
an active precursor component (e.g. active precursor polymer);
a physical activator agent (e.g. pH-modifier, e.g. a base, e.g. NaOH);
a chemical activator agent (e.g. an initiator); and
a contrast agent and/or visualisation agent (e.g. BaSO₄).

In a particular embodiment, the treatment composition comprises:
an active precursor component (e.g. active precursor polymer);
a physical activator agent (e.g. pH-modifier, e.g. a base, e.g. NaOH);
a chemical activator agent (e.g. an initiator); and
an accelerator (e.g. ascorbic acid).

In a particular embodiment, the treatment composition comprises:
an active precursor component (e.g. active precursor polymer);
a physical activator agent (e.g. pH-modifier, e.g. a base, e.g. NaOH);
a chemical activator agent (e.g. an initiator); and
an acid (e.g. ascorbic acid).

Suitably the accelerator may also be the acid.

In a particular embodiment, the treatment composition comprises:
an active precursor component (e.g. active precursor polymer);
a physical activator agent (e.g. pH-modifier, e.g. a base, e.g. NaOH);
a chemical activator agent (e.g. an initiator); and
an accelerator and/or acid (e.g. ascorbic acid).

In a particular embodiment, the treatment composition comprises:
an active precursor component (e.g. active precursor polymer);
a physical activator agent (e.g. pH-modifier, e.g. a base, e.g. NaOH);
a chemical activator agent (e.g. an initiator);
an accelerator and/or acid (e.g. ascorbic acid); and
a contrast agent and/or visualisation agent (e.g. BaSO₄).

Where the treatment composition comprises a contrast agent and/or visualisation agent, the treatment composition may further comprise one or more emulsification or solubilisation vehicles therefor, including, for example, one or more ingredients selected from a salt of a multidentate anion (e.g. citrate salt, e.g. sodium citrate), a polyol (e.g. a sugar, e.g. sorbitol, e.g. D-sorbitol), an antifoaming agent (e.g. simethicone), a hydrophilic polymer (e.g. a polyalkoxylene glycol polymer, e.g. a PEG, e.g. PEG 400), or any combination thereof.

In a particular embodiment, the treatment composition comprises:
1-30 wt % active precursor component (e.g. active precursor polymer); and
sufficient quantities of a physical activator agent (e.g. pH-modifier, e.g. a base, e.g. NaOH) to furnish a pH above pH 5.

In a particular embodiment, the treatment composition comprises:
1-30 wt % active precursor component (e.g. active precursor polymer); and
a contrast agent and/or visualisation agent (e.g. BaSO₄).

In a particular embodiment, the treatment composition comprises:
1-30 wt % an active precursor component (e.g. active precursor polymer);
sufficient quantities of a physical activator agent (e.g. pH-modifier, e.g. a base, e.g. NaOH) to furnish a pH above pH 5; and
a contrast agent and/or visualisation agent (e.g. BaSO₄).

In a particular embodiment, the treatment composition comprises:
1-30 wt % active precursor component (e.g. active precursor polymer); and
0.001-6 wt % chemical activator agent(s) (e.g. an initiator and/or accelerator).

In a particular embodiment, the treatment composition comprises:
1-30 wt % an active precursor component (e.g. active precursor polymer);
0.001-6 wt % chemical activator agent(s) (e.g. an initiator and/or accelerator); and
an accelerator (e.g. ascorbic acid).

In a particular embodiment, the treatment composition comprises:
1-30 wt % an active precursor component (e.g. active precursor polymer);
sufficient quantities of a physical activator agent (e.g. pH-modifier, e.g. a base, e.g. NaOH) to furnish a pH above pH 5; and
0.001-6 wt % chemical activator agent(s) (e.g. an initiator and/or accelerator).

In a particular embodiment, the treatment composition comprises:
1-30 wt % an active precursor component (e.g. active precursor polymer);
sufficient quantities of a physical activator agent (e.g. pH-modifier, e.g. a base, e.g. NaOH) to furnish a pH above pH 5;
0.001-6 wt % chemical activator agent(s) (e.g. an initiator and/or accelerator); and
a contrast agent and/or visualisation agent (e.g. BaSO₄).

In a particular embodiment, the treatment composition comprises:
1-30 wt % active precursor component (e.g. active precursor polymer);
sufficient quantities of a physical activator agent (e.g. pH-modifier, e.g. a base, e.g. NaOH) to furnish a pH above pH 5;
0.001-5 wt % initiator(s) (e.g. ammonium persulphate); and
0.0001-2 wt % accelerator(s) (e.g. ascorbic acid).

In a particular embodiment, the treatment composition comprises:
1-30 wt % active precursor component (e.g. active precursor polymer);
sufficient quantities of a physical activator agent (e.g. pH-modifier, e.g. a base, e.g. NaOH) to furnish a pH above pH 5;
0.001-6 wt % chemical activator agent(s) (e.g. an initiator and/or accelerator); and
0.0001-2 wt % acid (e.g. ascorbic acid).

Suitably the accelerator may also be the acid.

In a particular embodiment, the treatment composition comprises:
1-30 wt % active precursor component (e.g. active precursor polymer);
sufficient quantities of a physical activator agent (e.g. pH-modifier, e.g. a base, e.g. NaOH) to furnish a pH above pH 5;
0.001-5 wt % initiator(s) (e.g. ammonium persulphate); and
0.0001-2 wt % accelerator(s) (e.g. ascorbic acid).

In a particular embodiment, the treatment composition comprises:
1-30 wt % active precursor component (e.g. active precursor polymer);
sufficient quantities of a physical activator agent (e.g. pH-modifier, e.g. a base, e.g. NaOH) to furnish a pH above pH 5;
0.001-5 wt % initiator(s) (e.g. ammonium persulphate);
0.0001-2 wt % accelerator(s) (e.g. ascorbic acid); and
a contrast agent and/or visualisation agent (e.g. BaSO₄).

In a particular embodiment, the treatment composition comprises:
5-25 wt % active precursor component (e.g. active precursor polymer); and
sufficient quantities of a physical activator agent (e.g. pH-modifier, e.g. a base, e.g. NaOH) to furnish a pH above pH 6.

In a particular embodiment, the treatment composition comprises:
5-25 wt % active precursor component (e.g. active precursor polymer); and
a contrast agent and/or visualisation agent (e.g. BaSO₄).

In a particular embodiment, the treatment composition comprises:
5-25 wt % active precursor component (e.g. active precursor polymer);
sufficient quantities of a physical activator agent (e.g. pH-modifier, e.g. a base, e.g. NaOH) to furnish a pH above pH 6; and
a contrast agent and/or visualisation agent (e.g. BaSO₄).

In a particular embodiment, the treatment composition comprises:
5-25 wt % active precursor component (e.g. active precursor polymer); and
0.01-3 wt % chemical activator agent(s) (e.g. an initiator and/or accelerator).

In a particular embodiment, the treatment composition comprises:
5-25 wt % active precursor component (e.g. active precursor polymer);
0.01-3 wt % chemical activator agent(s) (e.g. an initiator and/or accelerator); and
an accelerator (e.g. ascorbic acid).

In a particular embodiment, the treatment composition comprises:
5-25 wt % active precursor component (e.g. active precursor polymer);
sufficient quantities of a physical activator agent (e.g. pH-modifier, e.g. a base, e.g. NaOH) to furnish a pH above pH 6; and
0.01-3 wt % chemical activator agent(s) (e.g. an initiator and/or accelerator).

In a particular embodiment, the treatment composition comprises: 5-25 wt % active precursor component (e.g. active precursor polymer);
sufficient quantities of a physical activator agent (e.g. pH-modifier, e.g. a base, e.g. NaOH) to furnish a pH above pH 6;
0.01-3 wt % chemical activator agent(s) (e.g. an initiator and/or accelerator); and
a contrast agent and/or visualisation agent (e.g. BaSO₄).

In a particular embodiment, the treatment composition comprises:
5-25 wt % active precursor component (e.g. active precursor polymer);
sufficient quantities of a physical activator agent (e.g. pH-modifier, e.g. a base, e.g. NaOH) to furnish a pH above pH 6;
0.01-3 wt % initiator(s) (e.g. ammonium persulphate); and
0.001-1 wt % accelerator(s) (e.g. ascorbic acid).

In a particular embodiment, the treatment composition comprises:
5-25 wt % active precursor component (e.g. active precursor polymer);
sufficient quantities of a physical activator agent (e.g. pH-modifier, e.g. a base, e.g. NaOH) to furnish a pH above pH 6;
0.01-3 wt % chemical activator agent(s) (e.g. an initiator and/or accelerator); and
0.001-1 wt % acid (e.g. ascorbic acid).

Suitably the accelerator may also be the acid.

In a particular embodiment, the treatment composition comprises:
5-25 wt % active precursor component (e.g. active precursor polymer);
sufficient quantities of a physical activator agent (e.g. pH-modifier, e.g. a base, e.g. NaOH) to furnish a pH above pH 6;
0.01-3 wt % initiator(s) (e.g. ammonium persulphate); and
0.001-1 wt % accelerator(s) (e.g. ascorbic acid).

In a particular embodiment, the treatment composition comprises:
5-25 wt % active precursor component (e.g. active precursor polymer);
sufficient quantities of a physical activator agent (e.g. pH-modifier, e.g. a base, e.g. NaOH) to furnish a pH above pH 6;
0.01-3 wt % initiator(s) (e.g. ammonium persulphate);
0.001-1 wt % accelerator(s) (e.g. ascorbic acid); and
a contrast agent and/or visualisation agent (e.g. BaSO₄).

In a particular embodiment, the treatment composition comprises:
10-20 wt % active precursor component (e.g. active precursor polymer); and
sufficient quantities of a physical activator agent (e.g. pH-modifier, e.g. a base, e.g. NaOH) to furnish a pH above pH 6.

In a particular embodiment, the treatment composition comprises:
10-20 wt % active precursor component (e.g. active precursor polymer); and
a contrast agent and/or visualisation agent (e.g. BaSO₄).

In a particular embodiment, the treatment composition comprises:
10-20 wt % active precursor component (e.g. active precursor polymer);
sufficient quantities of a physical activator agent (e.g. pH-modifier, e.g. a base, e.g. NaOH) to furnish a pH above pH 6; and
a contrast agent and/or visualisation agent (e.g. BaSO₄).

In a particular embodiment, the treatment composition comprises:
10-20 wt % active precursor component (e.g. active precursor polymer); and
0.1-1 wt % chemical activator agent(s) (e.g. an initiator and/or accelerator).

In a particular embodiment, the treatment composition comprises:
5-25 wt % active precursor component (e.g. active precursor polymer);
0.1-1 wt % chemical activator agent(s) (e.g. an initiator and/or accelerator); and
an accelerator (e.g. ascorbic acid).

In a particular embodiment, the treatment composition comprises:
10-20 wt % active precursor component (e.g. active precursor polymer);
sufficient quantities of a physical activator agent (e.g. pH-modifier, e.g. a base, e.g. NaOH) to furnish a pH above pH 6; and
0.1-1 wt % chemical activator agent(s) (e.g. an initiator and/or accelerator).

In a particular embodiment, the treatment composition comprises:
10-20 wt % active precursor component (e.g. active precursor polymer);
sufficient quantities of a physical activator agent (e.g. pH-modifier, e.g. a base, e.g. NaOH) to furnish a pH above pH 6;
0.1-1 wt % chemical activator agent(s) (e.g. an initiator and/or accelerator); and
a contrast agent and/or visualisation agent (e.g. BaSO₄).

In a particular embodiment, the treatment composition comprises:
10-20 wt % active precursor component (e.g. active precursor polymer);
sufficient quantities of a physical activator agent (e.g. pH-modifier, e.g. a base, e.g. NaOH) to furnish a pH above pH 6;
0.1-1 wt % initiator(s) (e.g. ammonium persulphate); and
0.01-0.5 wt % accelerator(s) (e.g. ascorbic acid).

In a particular embodiment, the treatment composition comprises:
10-20 wt % active precursor component (e.g. active precursor polymer);
sufficient quantities of a physical activator agent (e.g. pH-modifier, e.g. a base, e.g. NaOH) to furnish a pH above pH 6;
0.01-3 wt % chemical activator agent(s) (e.g. an initiator and/or accelerator); and
0.01-0.5 wt % acid (e.g. ascorbic acid).

Suitably the accelerator may also be the acid.

In a particular embodiment, the treatment composition comprises:
10-20 wt % active precursor component (e.g. active precursor polymer);
sufficient quantities of a physical activator agent (e.g. pH-modifier, e.g. a base, e.g. NaOH) to furnish a pH above pH 6;
0.1-1 wt % initiator(s) (e.g. ammonium persulphate); and
0.01-0.5 wt % accelerator(s) (e.g. ascorbic acid).

In a particular embodiment, the treatment composition comprises:
10-20 wt % active precursor component (e.g. active precursor polymer);
sufficient quantities of a physical activator agent (e.g. pH-modifier, e.g. a base, e.g. NaOH) to furnish a pH above pH 6;
0.1-1 wt % initiator(s) (e.g. ammonium persulphate);
0.01-0.5 wt % accelerator(s) (e.g. ascorbic acid); and
a contrast agent and/or visualisation agent (e.g. BaSO₄).

Suitably all treatment compositions comprise an aqueous medium, suitably water.

Suitably at least 50 wt % of the remaining balance (i.e. the amount required so that all ingredients together constitute 100 wt %) of ingredients of the treatment composition consists of water, suitably at least 60 wt % of the remaining balance, more suitably at least 70 wt % of the remaining balance, suitably at least 80 wt % of the remaining balance, suitably at least 90 wt % of the remaining balance, suitably at least 95 wt % of the remaining balance, more suitably all of the remaining balance consists of water.

Suitably the pH of the treatment composition is between pH 6 and pH 8, suitably between pH 7 and pH 8, more suitably between 7.2 and 7.6, most suitably about pH 7.4.

Suitably a corresponding post-treatment composition may be defined by and/or characterised by exactly the same ingredients and/or amounts of ingredients as for a treatment composition, except that the “active precursor component” is instead the “active component” (which is suitably a physically and/or chemically transformed active precursor component), and other ingredients (e.g. chemical activator agent(s), initiator(s), accelerator(s), physical activator agent(s), contrast agent(s)) may either be the same as the other ingredients or be a product derived therefrom. Suitably the active component of a post-treatment composition will be a transformed derivative of the active precursor component of the corresponding treatment composition (though suitably present in substantially a same or similar quantity), and any activator agents will within the post-treatment composition become products derived from the corresponding activator agents of the corresponding treatment composition.

As such, by way of example: In a particular embodiment, the post-treatment composition comprises:
an active component;
a physical activator agent (e.g. pH-modifier, e.g. a base, e.g. NaOH) or a product derived therefrom;
a chemical activator agent (e.g. an initiator) or a product derived therefrom;
an accelerator and/or acid (e.g. ascorbic acid) or a product derived therefrom; and
optionally a contrast agent and/or visualisation agent (e.g. BaSO₄) or a product derived therefrom.

By way of another example: In a particular embodiment, the post-treatment composition comprises:
10-20 wt % active component;
sufficient quantities of a physical activator agent (e.g. pH-modifier, e.g. a base, e.g. NaOH) to furnish a pH above pH 6 or a product derived therefrom;
0.1-1 wt % initiator(s) (e.g. ammonium persulphate) or a product derived therefrom;
0.01-0.5 wt % accelerator(s) (e.g. ascorbic acid) or a product derived therefrom; and
Optionally a contrast agent and/or visualisation agent (e.g. BaSO₄) or a product derived therefrom.

In a particular embodiment, the active precursor component of any, some, or all of the aforesaid embodiments of treatment composition, is a gellable component, suitably a gellable active precursor polymer, suitably a gellable polymeric particle, more suitably a microgel particle, more suitably a microgel particle comprising pre-grafted activatable moieties, most suitably a microgel particle comprising pre-grafted vinyl-containing moieties. Most suitably the active precursor component of any of the aforesaid embodiments is as defined herein. Suitably, the corresponding active component of any, some, or all of the aforesaid embodiments of corresponding post-treatment composition, is a gellable component, suitably a gellable polymer, suitably a gellable polymeric particle, more suitably a gellable microgel particle, more suitably a cross-linked and/or polymerised network of microgel particles, more suitably a cross-linked and/or polymerised network of microgel particles linked together via coupling (suitably free-radical coupling) of pre-grafted activatable moieties, most suitably a cross-linked and/or polymerised network of microgel particles linked together via coupling (suitably free-radical coupling) of pre-grafted vinyl-containing moieties. Most suitably the active component of any of the aforesaid embodiments is as defined herein.

In a particular embodiment, the chemical activator agent(s) of any, some, or all of the aforesaid embodiments of treatment composition, comprise an initiator, suitably a free-radical initiator, suitably ammonium presulphate. In a particular embodiment, the chemical activator agent(s) of any, some, or all of the aforesaid embodiments of treatment composition, comprise an accelerator, especially where the chemical activator agent(s) also comprise an initiator, wherein the accelerator is most suitably ascorbic acid. In corresponding post-treatment compositions, suitably the chemical activator agent(s) comprise the same aforesaid chemical activator agent(s) and/or a product derived therefrom (e.g. following free-radical reactions, oxidations, reductions, etc).

In a particular embodiment, the physical activator agent(s) of any, some, or all of the aforesaid embodiments of treatment composition, comprise a base, suitably an inorganic base, suitably an oxide or hydroxide salt, suitably a metal salt of an oxide or hydroxide, suitably an alkali or alkaline earth metal salt of an oxide or hydroxide, suitably sodium hydroxide. In corresponding post-treatment compositions, suitably the physical activator agent(s) comprise the same aforesaid physical activator agent(s) and/or a product derived therefrom (e.g. following acid-base reactions).

In a particular embodiment, the contrast agent and/or visualisation agent of any, some, or all of the aforesaid embodiments of treatment composition, comprise barium sulphate, suitably in conjunction with emulsification components to maintain barium sulphate in an injectable form.

### Particular Embodiments of Kits of Parts

In an embodiment:
the activatable composition comprises the active precursor component and an accelerator; and
the activator composition comprises an initiator; and pH-modifier; and optionally a contrast/visualisation agent.

Where the activator composition comprises a contrast agent and/or visualisation agent, the activator composition may further comprise one or more emulsification or solubilisation vehicles therefor, including, for example, one or more ingredients selected from a salt of a multidentate anion (e.g. citrate salt, e.g. sodium citrate), a polyol (e.g. a sugar, e.g. sorbitol, e.g. D-sorbitol), an antifoaming agent (e.g. simethicone), a hydrophilic polymer (e.g. a polyalkoxylene glycol polymer, e.g. a PEG, e.g. PEG 400), or any combination thereof.

In the embodiments that follow, the stated wt % of components relate to the wt % of component by total weight of the individual composition(s) in question, not to the total weight of a treatment composition formed by mixing the individual composition(s).

In a particular embodiment:
the activatable composition comprises 1-30 wt % active precursor component (e.g. active precursor polymer); and
the activator composition comprises 0.001-6 wt % chemical activator agent(s) (e.g. an initiator and/or accelerator).

In a particular embodiment:
the activatable composition comprises 1-30 wt % active precursor component (e.g. active precursor polymer), and 0.0001-2 wt % accelerator(s) (e.g. ascorbic acid); and
the activator composition comprises 0.001-5 wt % initiator(s) (e.g. ammonium persulphate).

In a particular embodiment:
the activatable composition comprises 1-30 wt % active precursor component (e.g. active precursor polymer); and
the activator composition comprises a pH-modifier, suitably sufficient pH-modifier to furnish a pH above pH 5 when the activatable and activator compositions are mixed.

In a particular embodiment:
the activatable composition comprises 1-30 wt % active precursor component (e.g. active precursor polymer); and
the activator composition comprises 0.001-6 wt % chemical activator agent(s) (e.g. an initiator and/or accelerator), and a pH-modifier, suitably sufficient pH-modifier to furnish a pH above pH 5 when the activatable and activator compositions are mixed.

In a particular embodiment:
the activatable composition comprises 1-30 wt % active precursor component (e.g. active precursor polymer), and 0.0001-2 wt % accelerator(s) (e.g. ascorbic acid); and
the activator composition comprises 0.001-5 wt % initiator(s) (e.g. ammonium persulphate), and a pH-modifier, suitably sufficient pH-modifier to furnish a pH above pH 5 when the activatable and activator compositions are mixed.

In a particular embodiment:
the activatable composition comprises 1-30 wt % active precursor component (e.g. active precursor polymer); and
the activator composition comprises a contrast agent and/or visualisation agent (e.g. BaSO₄).

In a particular embodiment:
the activatable composition comprises 1-30 wt % active precursor component (e.g. active precursor polymer); and
the activator composition comprises 0.001-6 wt % chemical activator agent(s) (e.g. an initiator and/or accelerator), and optionally a contrast agent and/or visualisation agent (e.g. BaSO₄).

In a particular embodiment:
the activatable composition comprises 1-30 wt % active precursor component (e.g. active precursor polymer); and
the activator composition comprises a pH-modifier, suitably sufficient pH-modifier to furnish a pH above pH 5 when the activatable and activator compositions are mixed, and optionally a contrast agent and/or visualisation agent (e.g. BaSO₄).

In a particular embodiment:
the activatable composition comprises 1-30 wt % active precursor component (e.g. active precursor polymer); and
the activator composition comprises 0.001-6 wt % chemical activator agent(s) (e.g. an initiator and/or accelerator), and a pH-modifier, suitably sufficient pH-modifier to furnish a pH above pH 5 when the activatable and activator compositions are mixed, and optionally a contrast agent and/or visualisation agent (e.g. BaSO₄).

In a particular embodiment:
the activatable composition comprises 1-30 wt % active precursor component (e.g. active precursor polymer), and 0.0001-2 wt % accelerator(s) (e.g. ascorbic acid); and
the activator composition comprises 0.001-5 wt % initiator(s) (e.g. ammonium persulphate), and optionally a contrast agent and/or visualisation agent (e.g. BaSO₄).

In a particular embodiment:
the activatable composition comprises 1-30 wt % active precursor component (e.g. active precursor polymer), and 0.0001-2 wt % accelerator(s) (e.g. ascorbic acid); and
the activator composition comprises 0.001-5 wt % initiator(s) (e.g. ammonium persulphate), a contrast agent and/or visualisation agent (e.g. BaSO₄), and a pH-modifier, suitably sufficient pH-modifier to furnish a pH above pH 5 when the activatable and activator compositions are mixed.

In a particular embodiment:
the activatable composition comprises 5-25 wt % active precursor component (e.g. active precursor polymer); and
the activator composition comprises 0.01-3 wt % chemical activator agent(s) (e.g. an initiator and/or accelerator).

In a particular embodiment:
the activatable composition comprises 5-25 wt % active precursor component (e.g. active precursor polymer), and 0.001-2 wt % accelerator(s) (e.g. ascorbic acid); and
the activator composition comprises 0.01-3 wt % initiator(s) (e.g. ammonium persulphate).

In a particular embodiment:
the activatable composition comprises 5-25 wt % active precursor component (e.g. active precursor polymer); and
the activator composition comprises a pH-modifier, suitably sufficient pH-modifier to furnish a pH above pH 6 when the activatable and activator compositions are mixed.

In a particular embodiment:
the activatable composition comprises 5-25 wt % active precursor component (e.g. active precursor polymer); and
the activator composition comprises 0.01-3 wt % chemical activator agent(s) (e.g. an initiator and/or accelerator), and a pH-modifier, suitably sufficient pH-modifier to furnish a pH above pH 6 when the activatable and activator compositions are mixed.

In a particular embodiment:
the activatable composition comprises 5-25 wt % active precursor component (e.g. active precursor polymer), and 0.001-2 wt % accelerator(s) (e.g. ascorbic acid); and
the activator composition comprises 0.01-3 wt % initiator(s) (e.g. ammonium persulphate), and a pH-modifier, suitably sufficient pH-modifier to furnish a pH above pH 6 when the activatable and activator compositions are mixed.

In a particular embodiment:
the activatable composition comprises 5-25 wt % active precursor component (e.g. active precursor polymer); and
the activator composition comprises a contrast agent and/or visualisation agent (e.g. BaSO₄).

In a particular embodiment:
the activatable composition comprises 5-25 wt % active precursor component (e.g. active precursor polymer); and
the activator composition comprises 0.01-3 wt % chemical activator agent(s) (e.g. an initiator and/or accelerator), and a contrast agent and/or visualisation agent (e.g. BaSO₄).

In a particular embodiment:
the activatable composition comprises 5-25 wt % active precursor component (e.g. active precursor polymer); and
the activator composition comprises a contrast agent and/or visualisation agent (e.g. BaSO₄), and a pH-modifier, suitably sufficient pH-modifier to furnish a pH above pH 6 when the activatable and activator compositions are mixed.

In a particular embodiment:
the activatable composition comprises 5-25 wt % active precursor component (e.g. active precursor polymer); and
the activator composition comprises a contrast agent and/or visualisation agent (e.g. BaSO₄), 0.01-3 wt % chemical activator agent(s) (e.g. an initiator and/or accelerator), and
a pH-modifier, suitably sufficient pH-modifier to furnish a pH above pH 6 when the activatable and activator compositions are mixed.

In a particular embodiment:
the activatable composition comprises 5-25 wt % active precursor component (e.g. active precursor polymer), and 0.001-2 wt % accelerator(s) (e.g. ascorbic acid); and
the activator composition comprises 0.01-3 wt % initiator(s) (e.g. ammonium persulphate), and optionally a contrast agent and/or visualisation agent (e.g. BaSO₄).

In a particular embodiment:
the activatable composition comprises 5-25 wt % active precursor component (e.g. active precursor polymer), and 0.001-2 wt % accelerator(s) (e.g. ascorbic acid); and
the activator composition comprises 0.01-3 wt % initiator(s) (e.g. ammonium persulphate), a contrast agent and/or visualisation agent (e.g. BaSO₄), and a pH-modifier, suitably sufficient pH-modifier to furnish a pH above pH 6 when the activatable and activator compositions are mixed.

In a particular embodiment:
the activatable composition comprises 15-20 wt % active precursor component (e.g. active precursor polymer); and
the activator composition comprises 1-2 wt % chemical activator agent(s) (e.g. an initiator and/or accelerator).

In a particular embodiment:
the activatable composition comprises 15-20 wt % active precursor component (e.g. active precursor polymer), and 0.05-1 wt % accelerator(s) (e.g. ascorbic acid); and
the activator composition comprises 1-2 wt % initiator(s) (e.g. ammonium persulphate).

In a particular embodiment:
the activatable composition comprises 15-20 wt % active precursor component (e.g. active precursor polymer); and
the activator composition comprises a pH-modifier, suitably sufficient pH-modifier to furnish a pH above pH 6 when the activatable and activator compositions are mixed.

In a particular embodiment:
the activatable composition comprises 15-20 wt % active precursor component (e.g. active precursor polymer); and
the activator composition comprises 1-2 wt % chemical activator agent(s) (e.g. an initiator and/or accelerator), and a pH-modifier, suitably sufficient pH-modifier to furnish a pH above pH 6 when the activatable and activator compositions are mixed.

In a particular embodiment:
the activatable composition comprises 15-20 wt % active precursor component (e.g. active precursor polymer), and 0.05-1 wt % accelerator(s) (e.g. ascorbic acid); and
the activator composition comprises 1-2 wt % initiator(s) (e.g. ammonium persulphate), and a pH-modifier, suitably sufficient pH-modifier to furnish a pH above pH 6 when the activatable and activator compositions are mixed.

In a particular embodiment:
the activatable composition comprises 15-20 wt % active precursor component (e.g. active precursor polymer); and
the activator composition comprises a contrast agent and/or visualisation agent (e.g. BaSO₄).

In a particular embodiment:
the activatable composition comprises 15-20 wt % active precursor component (e.g. active precursor polymer); and
the activator composition comprises 1-2 wt % chemical activator agent(s) (e.g. an initiator and/or accelerator), and a contrast agent and/or visualisation agent (e.g. BaSO₄).

In a particular embodiment:
the activatable composition comprises 15-20 wt % active precursor component (e.g. active precursor polymer); and
the activator composition comprises a contrast agent and/or visualisation agent (e.g. BaSO₄), and a pH-modifier, suitably sufficient pH-modifier to furnish a pH above pH 6 when the activatable and activator compositions are mixed.

In a particular embodiment:
the activatable composition comprises 15-20 wt % active precursor component (e.g. active precursor polymer); and
the activator composition comprises a contrast agent and/or visualisation agent (e.g. BaSO₄), 1-2 wt % chemical activator agent(s) (e.g. an initiator and/or accelerator), and a pH-modifier, suitably sufficient pH-modifier to furnish a pH above pH 6 when the activatable and activator compositions are mixed.

In a particular embodiment:
the activatable composition comprises 15-20 wt % active precursor component (e.g. active precursor polymer), and 0.05-1 wt % accelerator(s) (e.g. ascorbic acid); and
the activator composition comprises 1-2 wt % initiator(s) (e.g. ammonium persulphate), and a contrast agent and/or visualisation agent (e.g. BaSO₄).

In a particular embodiment:
the activatable composition comprises 15-20 wt % active precursor component (e.g. active precursor polymer), and 0.05-1 wt % accelerator(s) (e.g. ascorbic acid); and
the activator composition comprises 1-2 wt % initiator(s) (e.g. ammonium persulphate), a contrast agent and/or visualisation agent (e.g. BaSO₄), and a pH-modifier, suitably sufficient pH-modifier to furnish a pH above pH 6 when the activatable and activator compositions are mixed.

As per the aforementioned specific embodiments relating to the treatment composition, suitably all compositions comprise an aqueous medium, suitably water. Suitably at least 50 wt % of the remaining balance (i.e. the amount required so that all ingredients together constitute 100 wt %) of ingredients of the composition consists of water, suitably at least 60 wt % of the remaining balance, more suitably at least 70 wt % of the remaining balance, suitably at least 80 wt % of the remaining balance, suitably at least 90 wt % of the remaining balance, suitably at least 95 wt % of the remaining balance, more suitably all of the remaining balance consists of water.

Suitably a pH modifier is present in an amount sufficient to furnish the treatment composition with a pH between pH 7 and pH 8, more suitably between 7.2 and 7.6, most suitably about pH 7.4.

Other factors pertinent to the aforementioned specific embodiments relating to the treatment composition, such as the nature of the active precursor component, the activator agents, and contrast/visualisation agents, are also relevant for the kit of parts.

### Additional Compositions and Kits of the Invention

According to a further aspect of the present invention there is provided an activatable composition, suitably as defined hereinbefore or hereinafter. The activatable composition suitably comprises an active precursor component, which is suitably activatable to physically (e.g. gel/swell) and/or chemically (e.g. cure/crosslink) transform, especially in situ when administered to a target location.

According to a further aspect of the present invention there is provided an activator composition, suitably as defined hereinbefore or hereinafter. The activator composition suitably comprises one or more activator agents, suitably which when mixed with the activatable composition, cause the active precursor component to physically (e.g. gel/swell) and/or chemically (e.g. cure/crosslink) transform, especially in situ when administered to a target location.

According to a further aspect of the present invention there is provided a kit comprising an activatable composition, suitably as defined hereinbefore or hereinafter, and an activator composition, suitably as defined hereinbefore or hereinafter, and optionally a set of instructions describing how to use the kit. The kits is suitably used by mixing the activatable and activator composition to form a treatment composition immediately prior to its administration to a target location. Suitably the volumetric ratio of activatable composition to activator composition is between 1-30:1, suitably between 2-20:1, suitably 4-15:1, suitably 8-12:1, most suitably about 10:1.

According to a further aspect of the present invention there is provided a treatment composition, suitably as defined hereinbefore or hereinafter. The treatment composition may be formed by mixing together the aforementioned activatable composition and activator composition, suitably to effect a physical and/or chemical transformation of an active precursor component present within the activatable composition.

According to a further aspect of the present invention there is provided a post-treatment composition, suitably as defined hereinbefore or hereinafter. The post-treatment composition is suitably be formed by mixing together the aforementioned activatable composition and activator composition, to initially provide a treatment composition, which treatment composition suitably undergoes a physical and/or chemical transformation, suitably based on a physical and/or chemical transformation of an active precursor component present within the initial activatable composition. As such, the post-treatment composition is suitably formed by allowing the treatment composition to cure or otherwise react.

According to a further aspect of the present invention there is provided a delivery device comprising: a container for an activatable composition; a container for an activator composition; a mixing chamber fluidly connectable to each of the containers; a dispensing outlet fluidly connectable to the mixing chamber; and a delivery mechanism operable to cause both activatable composition and activator composition to exit their respective containers into the mixing chamber wherein the activatable composition and activator composition mix together to form a treatment composition before the treatment composition is dispensed from the dispensing outlet. The containers suitably contain their respective activatable composition and activator composition. The volumetric ratio of activatable composition to activator composition is suitably between 1-30:1, suitably between 2-20:1, suitably 4-15:1, suitably 8-12:1, most suitably about 10:1. The delivery device is suitably a double-barreled syringe, and the delivery mechanism suitably comprises a syringe plunger. The dispensing outlet suitably comprises or is otherwise fitted with a needle, suitably as defined elsewhere herein.

### Specific Embodiments

The activatable composition suitably comprises: an active precursor component. The active precursor component is suitably: 10-20 wt % active precursor component. The active precursor component is suitably: crosslinkable microgel particles. The active precursor component is suitably: crosslinkable microgel particles with 2-8 mol. % of all monomers thereof functionalised with a crosslinkable moiety, such as a moiety comprise a terminal alkene. The active precursor component is suitably: crosslinkable microgel particles having an average particle size between 30 and 90 nm. The active precursor component is suitably: 10-20 wt % crosslinkable microgel particles. The active precursor component is suitably: crosslinkable microgel particles comprising poly(MMA/MAA/EGDMA) cores surface-functionalised with GMA, wherein the cores comprise comprises about 60-70% MMA, about 30-40% MAA and 0.1 to 1% EGDMA based on the total monomer mass. The active precursor component is suitably: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass. The active precursor component is suitably: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA.

In addition to the active precursor component, the activatable composition suitably comprises: an accelerator. Suitably, the accelerator is: 0.01-0.2 wt % accelerator. Suitably, the accelerator is: ascorbic acid (or a salt thereof). Suitably, the accelerator is: 0.01-0.2 wt % ascorbic acid (or a salt thereof). Suitably, the accelerator is: 0.05-0.15 wt % ascorbic acid (or a salt thereof).

Suitably the activatable composition: is characterised by a pH between 5 and 6. Suitably the activatable composition: is characterised by a pH between 5.3 and 5.7.

In an embodiment, the activatable composition comprises: an active precursor component; and an accelerator. In an embodiment, the activatable composition comprises: an active precursor component; and 0.01-0.2 wt % accelerator. In an embodiment, the activatable composition comprises: an active precursor component; and ascorbic acid (or a salt thereof). In an embodiment, the activatable composition comprises: an active precursor component; and 0.01-0.2 wt % ascorbic acid (or a salt thereof). In an embodiment, the activatable composition comprises: an active precursor component; and 0.05-0.15 wt % ascorbic acid (or a salt thereof). In an embodiment, the activatable composition comprises: an active precursor component; an accelerator; and is characterised by a pH between 5 and 6. In an embodiment, the activatable composition comprises: an active precursor component; 0.01-0.2 wt % accelerator; and is characterised by a pH between 5 and 6. In an embodiment, the activatable composition comprises: an active precursor component; ascorbic acid (or a salt thereof); and is characterised by a pH between 5 and 6. In an embodiment, the activatable composition comprises: an active precursor component; 0.01-0.2 wt % ascorbic acid (or a salt thereof); and is characterised by a pH between 5 and 6. In an embodiment, the activatable composition comprises: an active precursor component; 0.05-0.15 wt % ascorbic acid (or a salt thereof); and is characterised by a pH between 5 and 6. In an embodiment, the activatable composition comprises: an active precursor component; an accelerator; and is characterised by a pH between 5.3 and 5.7. In an embodiment, the activatable composition comprises: an active precursor component; 0.01-0.2 wt % accelerator; and is characterised by a pH between 5.3 and 5.7. In an embodiment, the activatable composition comprises: an active precursor component; ascorbic acid (or a salt thereof); and is characterised by a pH between 5.3 and 5.7. In an embodiment, the activatable composition comprises: an active precursor component; 0.01-0.2 wt % ascorbic acid (or a salt thereof); and is characterised by a pH between 5.3 and 5.7. In an embodiment, the activatable composition comprises: an active precursor component; 0.05-0.15 wt % ascorbic acid (or a salt thereof); and is characterised by a pH between 5.3 and 5.7.

In an embodiment, the activatable composition comprises: an active precursor component; and an accelerator. In an embodiment, the activatable composition comprises: 10-20 wt % active precursor component; and an accelerator. In an embodiment, the activatable composition comprises: crosslinkable microgel particles; and an accelerator. In an embodiment, the activatable composition comprises: crosslinkable microgel particles with 2-8 mol. % of all monomers thereof functionalised with a crosslinkable moiety, such as a moiety comprise a terminal alkene; and an accelerator. In an embodiment, the activatable composition comprises: crosslinkable microgel particles having an average particle size between 30 and 90 nm; and an accelerator. In an embodiment, the activatable composition comprises: 10-20 wt % crosslinkable microgel particles; and an accelerator. In an embodiment, the activatable composition comprises: crosslinkable microgel particles comprising poly(MMA/MAA/EGDMA) cores surface-functionalised with GMA, wherein the cores comprise comprises about 60-70% MMA, about 30-40% MAA and 0.1 to 1% EGDMA based on the total monomer mass; and an accelerator. In an embodiment, the activatable composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass; and an accelerator. In an embodiment, the activatable composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; and an accelerator. In an embodiment, the activatable composition comprises: an active precursor component; an accelerator; and is characterised by a pH between 5 and 6. In an embodiment, the activatable composition comprises: 10-20 wt % active precursor component; an accelerator; and is characterised by a pH between 5 and 6. In an embodiment, the activatable composition comprises: crosslinkable microgel particles; an accelerator; and is characterised by a pH between 5 and 6. In an embodiment, the activatable composition comprises: crosslinkable microgel particles with 2-8 mol. % of all monomers thereof functionalised with a crosslinkable moiety, such as a moiety comprise a terminal alkene; an accelerator; and is characterised by a pH between 5 and 6. In an embodiment, the activatable composition comprises: crosslinkable microgel particles having an average particle size between 30 and 90 nm; an accelerator; and is characterised by a pH between 5 and 6. In an embodiment, the activatable composition comprises: 10-20 wt % crosslinkable microgel particles; an accelerator; and is characterised by a pH between 5 and 6. In an embodiment, the activatable composition comprises: crosslinkable microgel particles comprising poly(MMA/MAA/EGDMA) cores surface-functionalised with GMA, wherein the cores comprise comprises about 60-70% MMA, about 30-40% MAA and 0.1 to 1% EGDMA based on the total monomer mass; an accelerator; and is characterised by a pH between 5 and 6. In an embodiment, the activatable composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass; an accelerator; and is characterised by a pH between 5 and 6. In an embodiment, the activatable composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; and is characterised by a pH between 5 and 6. In an embodiment, the activatable composition comprises: an active precursor component; an accelerator; and is characterised by a pH between 5.3 and 5.7. In an embodiment, the activatable composition comprises: 10-20 wt % active precursor component; an accelerator; and is characterised by a pH between 5.3 and 5.7. In an embodiment, the activatable composition comprises: crosslinkable microgel particles; an accelerator; and is characterised by a pH between 5.3 and 5.7. In an embodiment, the activatable composition comprises: crosslinkable microgel particles with 2-8 mol. % of all monomers thereof functionalised with a crosslinkable moiety, such as a moiety comprise a terminal alkene; an accelerator; and is characterised by a pH between 5.3 and 5.7. In an embodiment, the activatable composition comprises: crosslinkable microgel particles having an average particle size between 30 and 90 nm; an accelerator; and is characterised by a pH between 5.3 and 5.7. In an embodiment, the activatable composition comprises: 10-20 wt % crosslinkable microgel particles; an accelerator; and is characterised by a pH between 5.3 and 5.7. In an embodiment, the activatable composition comprises: crosslinkable microgel particles comprising poly(MMA/MAA/EGDMA) cores surface-functionalised with GMA, wherein the cores comprise comprises about 60-70% MMA, about 30-40% MAA and 0.1 to 1% EGDMA based on the total monomer mass; an accelerator; and is characterised by a pH between 5.3 and 5.7. In an embodiment, the activatable composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass; an accelerator; and is characterised by a pH between 5.3 and 5.7. In an embodiment, the activatable composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; and is characterised by a pH between 5.3 and 5.7.

In an embodiment, the activatable composition comprises: crosslinkable microgel particles; and ascorbic acid (or a salt thereof). In an embodiment, the activatable composition comprises: 10-20 wt % crosslinkable microgel particles; and ascorbic acid (or a salt thereof). In an embodiment, the activatable composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; and ascorbic acid (or a salt thereof). In an embodiment, the activatable composition comprises: crosslinkable microgel particles; and 0.05-0.15 wt % ascorbic acid (or a salt thereof). In an embodiment, the activatable composition comprises: 10-20 wt % crosslinkable microgel particles; and 0.05-0.15 wt % ascorbic acid (or a salt thereof). In an embodiment, the activatable composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; and 0.05-0.15 wt % ascorbic acid (or a salt thereof).

The activator composition suitably comprises an initiator. The initiator suitably is: 1-5 wt % intiator. The initiator suitably is: ammonium persulfate. The initiator suitably is: 1-5 wt % ammonium persulfate. The initiator suitably is: 2.5-3.5 wt % ammonium persulfate. The initiator suitably is: 0.05-0.5 wt % initiator. The initiator suitably is: 0.05-0.5 wt % ammonium persulfate. The initiator suitably is: 0.2-0.4 wt % ammonium persulfate.

The activator composition suitably comprises a pH modifier. The pH modifier suitably is: a basifying or alkaline pH modifier. The pH modifier suitably is: sodium hydroxide. The pH modifier suitably is: 2-5 M sodium hydroxide. The pH modifier suitably is: 5-25 wt % NaOH. The pH modifier suitably is: 0.1-3 wt % NaOH. The pH modifier suitably is: 0.5-2 wt % NaOH.

The activator composition suitably comprises an initiator and a pH modifier. The activator composition is suitably characterised by a pH between pH 11 and pH 14, most suitably about pH 13.

In an embodiment, the activator composition comprises: an initiator; and a basifying or alkaline pH modifier. In an embodiment, the activator composition comprises: an initiator; and sodium hydroxide. In an embodiment, the activator composition comprises: an initiator; and 2-5 M sodium hydroxide. In an embodiment, the activator composition comprises: an initiator; and 5-25 wt % NaOH. In an embodiment, the activator composition comprises: an initiator; and 0.1-3 wt % NaOH. In an embodiment, the activator composition comprises: an initiator; and 0.5-2 wt % NaOH. In an embodiment, the activator composition comprises: an initiator; a pH modifier; and is characterised by a pH between 11 and 14. In an embodiment, the activator composition comprises: an initiator; a basifying or alkaline pH modifier; and is characterised by a pH between 11 and 14. In an embodiment, the activator composition comprises: an initiator; sodium hydroxide; and is characterised by a pH between 11 and 14. In an embodiment, the activator composition comprises: an initiator; 2-5 M sodium hydroxide; and is characterised by a pH between 11 and 14. In an embodiment, the activator composition comprises: an initiator; 5-25 wt % NaOH; and is characterised by a pH between 11 and 14. In an embodiment, the activator composition comprises: an initiator; 0.1-3 wt % NaOH; and is characterised by a pH between 11 and 14. In an embodiment, the activator composition comprises: an initiator; 0.5-2 wt % NaOH; and is characterised by a pH between 11 and 14.

In an embodiment, the activator composition comprises: 1-5 wt % intiator; and a pH modifier. In an embodiment, the activator composition comprises: ammonium persulfate; and a pH modifier. In an embodiment, the activator composition comprises: 1-5 wt % ammonium persulfate; and a pH modifier. In an embodiment, the activator composition comprises: 2.5-3.5 wt % ammonium persulfate; and a pH modifier. In an embodiment, the activator composition comprises: 0.05-0.5 wt % initiator; and a pH modifier. In an embodiment, the activator composition comprises: 0.05-0.5 wt % ammonium persulfate; and a pH modifier. In an embodiment, the activator composition comprises: 0.2-0.4 wt % ammonium persulfate; and a pH modifier. In an embodiment, the activator composition comprises: an initiator; a pH modifier; and is characterised by a pH between 11 and 14. In an embodiment, the activator composition comprises: 1-5 wt % intiator; a pH modifier; and is characterised by a pH between 11 and 14. In an embodiment, the activator composition comprises: ammonium persulfate; a pH modifier; and is characterised by a pH between 11 and 14. In an embodiment, the activator composition comprises: 1-5 wt % ammonium persulfate; a pH modifier; and is characterised by a pH between 11 and 14. In an embodiment, the activator composition comprises: 2.5-3.5 wt % ammonium persulfate; a pH modifier; and is characterised by a pH between 11 and 14. In an embodiment, the activator composition comprises: 0.05-0.5 wt % initiator; a pH modifier; and is characterised by a pH between 11 and 14. In an embodiment, the activator composition comprises: 0.05-0.5 wt % ammonium persulfate; a pH modifier; and is characterised by a pH between 11 and 14. In an embodiment, the activator composition comprises: 0.2-0.4 wt % ammonium persulfate; a pH modifier; and is characterised by a pH between 11 and 14.

In an embodiment, the activator composition comprises: ammonium persulfate; and sodium hydroxide. In an embodiment, the activator composition comprises: 2.5-3.5 wt % ammonium persulfate; and sodium hydroxide. In an embodiment, the activator composition comprises: ammonium persulfate; and 5-25 wt % NaOH. In an embodiment, the activator composition comprises: 2.5-3.5 wt % ammonium persulfate; and 5-25 wt % NaOH. In an embodiment, the activator composition comprises: ammonium persulfate; sodium hydroxide; and is characterised by a pH between 11 and 14. In an embodiment, the activator composition comprises: 2.5-3.5 wt % ammonium persulfate; sodium hydroxide; and is characterised by a pH between 11 and 14. In an embodiment, the activator composition comprises: ammonium persulfate; 5-25 wt % NaOH; and is characterised by a pH between 11 and 14. In an embodiment, the activator composition comprises: 2.5-3.5 wt % ammonium persulfate; 5-25 wt % NaOH; and is characterised by a pH between 11 and 14.

The activatable composition is suitably a colloidal suspension or a colloidal dispersion, suitably an aqueous colloidal suspension or aqueous colloidal dispersion. The activator composition is suitably an solution, suitably an aqueous solution.

Suitably, the treatment composition is formed by mixing together the activatable composition and activator composition in a volumetric ratio of activatable composition to activator composition of between 1-30:1, suitably between 2-20:1, suitably 4-15:1, suitably 8-12:1, most suitably about 10:1. The treatment composition suitably has a pH between 6.5 and 8, more suitably between 7 and 7.9, more suitably between 7.2 and 7.6, most suitably about pH 7.4.

In an embodiment, the treatment composition comprises: an active precursor component; an initiator; a pH modifier; and is characterised by a pH between 6.5 and 8. In an embodiment, the treatment composition comprises: crosslinkable microgel particles; an initiator; a pH modifier; and is characterised by a pH between 6.5 and 8. In an embodiment, the treatment composition comprises: 10-20 wt % crosslinkable microgel particles; an initiator; a pH modifier; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an initiator; a pH modifier; and is characterised by a pH between 6.5 and 8. In an embodiment, the treatment composition comprises: an active precursor component; ammonium persulfate; a pH modifier; and is characterised by a pH between 6.5 and 8. In an embodiment, the treatment composition comprises: crosslinkable microgel particles; ammonium persulfate; a pH modifier; and is characterised by a pH between 6.5 and 8. In an embodiment, the treatment composition comprises: 10-20 wt % crosslinkable microgel particles; ammonium persulfate; a pH modifier; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ammonium persulfate; a pH modifier; and is characterised by a pH between 6.5 and 8. In an embodiment, the treatment composition comprises: an active precursor component; 0.2-0.4 wt % ammonium persulfate; a pH modifier; and is characterised by a pH between 6.5 and 8. In an embodiment, the treatment composition comprises: crosslinkable microgel particles; 0.2-0.4 wt % ammonium persulfate; a pH modifier; and is characterised by a pH between 6.5 and 8. In an embodiment, the treatment composition comprises: 10-20 wt % crosslinkable microgel particles; 0.2-0.4 wt % ammonium persulfate; a pH modifier; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.2-0.4 wt % ammonium persulfate; a pH modifier; and is characterised by a pH between 6.5 and 8. In an embodiment, the treatment composition comprises: an active precursor component; an initiator; sodium hydroxide; and is characterised by a pH between 6.5 and 8. In an embodiment, the treatment composition comprises: crosslinkable microgel particles; an initiator; sodium hydroxide; and is characterised by a pH between 6.5 and 8. In an embodiment, the treatment composition comprises: 10-20 wt % crosslinkable microgel particles; an initiator; sodium hydroxide; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an initiator; sodium hydroxide; and is characterised by a pH between 6.5 and 8. In an embodiment, the treatment composition comprises: an active precursor component; ammonium persulfate; sodium hydroxide; and is characterised by a pH between 6.5 and 8. In an embodiment, the treatment composition comprises: crosslinkable microgel particles; ammonium persulfate; sodium hydroxide; and is characterised by a pH between 6.5 and 8. In an embodiment, the treatment composition comprises: 10-20 wt % crosslinkable microgel particles; ammonium persulfate; sodium hydroxide; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ammonium persulfate; sodium hydroxide; and is characterised by a pH between 6.5 and 8. In an embodiment, the treatment composition comprises: an active precursor component; 0.2-0.4 wt % ammonium persulfate; sodium hydroxide; and is characterised by a pH between 6.5 and 8. In an embodiment, the treatment composition comprises: crosslinkable microgel particles; 0.2-0.4 wt % ammonium persulfate; sodium hydroxide; and is characterised by a pH between 6.5 and 8. In an embodiment, the treatment composition comprises: 10-20 wt % crosslinkable microgel particles; 0.2-0.4 wt % ammonium persulfate; sodium hydroxide; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.2-0.4 wt % ammonium persulfate; sodium hydroxide; and is characterised by a pH between 6.5 and 8. In an embodiment, the treatment composition comprises: an active precursor component; an initiator; 0.5-2 wt % NaOH; and is characterised by a pH between 6.5 and 8. In an embodiment, the treatment composition comprises: crosslinkable microgel particles; an initiator; 0.5-2 wt % NaOH; and is characterised by a pH between 6.5 and 8. In an embodiment, the treatment composition comprises: 10-20 wt % crosslinkable microgel particles; an initiator; 0.5-2 wt % NaOH; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an initiator; 0.5-2 wt % NaOH; and is characterised by a pH between 6.5 and 8. In an embodiment, the treatment composition comprises: an active precursor component; ammonium persulfate; 0.5-2 wt % NaOH; and is characterised by a pH between 6.5 and 8. In an embodiment, the treatment composition comprises: crosslinkable microgel particles; ammonium persulfate; 0.5-2 wt % NaOH; and is characterised by a pH between 6.5 and 8. In an embodiment, the treatment composition comprises: 10-20 wt % crosslinkable microgel particles; ammonium persulfate; 0.5-2 wt % NaOH; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ammonium persulfate; 0.5-2 wt % NaOH; and is characterised by a pH between 6.5 and 8. In an embodiment, the treatment composition comprises: an active precursor component; 0.2-0.4 wt % ammonium persulfate; 0.5-2 wt % NaOH; and is characterised by a pH between 6.5 and 8. In an embodiment, the treatment composition comprises: crosslinkable microgel particles; 0.2-0.4 wt % ammonium persulfate; 0.5-2 wt % NaOH; and is characterised by a pH between 6.5 and 8. In an embodiment, the treatment composition comprises: 10-20 wt % crosslinkable microgel particles; 0.2-0.4 wt % ammonium persulfate; 0.5-2 wt % NaOH; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.2-0.4 wt % ammonium persulfate; 0.5-2 wt % NaOH; and is characterised by a pH between 6.5 and 8. In an embodiment, the treatment composition comprises: an active precursor component; an initiator; a pH modifier; and is characterised by a pH between 7.2 and 7.6. In an embodiment, the treatment composition comprises: crosslinkable microgel particles; an initiator; a pH modifier; and is characterised by a pH between 7.2 and 7.6. In an embodiment, the treatment composition comprises: 10-20 wt % crosslinkable microgel particles; an initiator; a pH modifier; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an initiator; a pH modifier; and is characterised by a pH between 7.2 and 7.6. In an embodiment, the treatment composition comprises: an active precursor component; ammonium persulfate; a pH modifier; and is characterised by a pH between 7.2 and 7.6. In an embodiment, the treatment composition comprises: crosslinkable microgel particles; ammonium persulfate; a pH modifier; and is characterised by a pH between 7.2 and 7.6. In an embodiment, the treatment composition comprises: 10-20 wt % crosslinkable microgel particles; ammonium persulfate; a pH modifier; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ammonium persulfate; a pH modifier; and is characterised by a pH between 7.2 and 7.6. In an embodiment, the treatment composition comprises: an active precursor component; 0.2-0.4 wt % ammonium persulfate; a pH modifier; and is characterised by a pH between 7.2 and 7.6. In an embodiment, the treatment composition comprises: crosslinkable microgel particles; 0.2-0.4 wt % ammonium persulfate; a pH modifier; and is characterised by a pH between 7.2 and 7.6. In an embodiment, the treatment composition comprises: 10-20 wt % crosslinkable microgel particles; 0.2-0.4 wt % ammonium persulfate; a pH modifier; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.2-0.4 wt % ammonium persulfate; a pH modifier; and is characterised by a pH between 7.2 and 7.6. In an embodiment, the treatment composition comprises: an active precursor component; an initiator; sodium hydroxide; and is characterised by a pH between 7.2 and 7.6. In an embodiment, the treatment composition comprises: crosslinkable microgel particles; an initiator; sodium hydroxide; and is characterised by a pH between 7.2 and 7.6. In an embodiment, the treatment composition comprises: 10-20 wt % crosslinkable microgel particles; an initiator; sodium hydroxide; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an initiator; sodium hydroxide; and is characterised by a pH between 7.2 and 7.6. In an embodiment, the treatment composition comprises: an active precursor component; ammonium persulfate; sodium hydroxide; and is characterised by a pH between 7.2 and 7.6. In an embodiment, the treatment composition comprises: crosslinkable microgel particles; ammonium persulfate; sodium hydroxide; and is characterised by a pH between 7.2 and 7.6. In an embodiment, the treatment composition comprises: 10-20 wt % crosslinkable microgel particles; ammonium persulfate; sodium hydroxide; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ammonium persulfate; sodium hydroxide; and is characterised by a pH between 7.2 and 7.6. In an embodiment, the treatment composition comprises: an active precursor component; 0.2-0.4 wt % ammonium persulfate; sodium hydroxide; and is characterised by a pH between 7.2 and 7.6. In an embodiment, the treatment composition comprises: crosslinkable microgel particles; 0.2-0.4 wt % ammonium persulfate; sodium hydroxide; and is characterised by a pH between 7.2 and 7.6. In an embodiment, the treatment composition comprises: 10-20 wt % crosslinkable microgel particles; 0.2-0.4 wt % ammonium persulfate; sodium hydroxide; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.2-0.4 wt % ammonium persulfate; sodium hydroxide; and is characterised by a pH between 7.2 and 7.6. In an embodiment, the treatment composition comprises: an active precursor component; an initiator; 0.5-2 wt % NaOH; and is characterised by a pH between 7.2 and 7.6. In an embodiment, the treatment composition comprises: crosslinkable microgel particles; an initiator; 0.5-2 wt % NaOH; and is characterised by a pH between 7.2 and 7.6. In an embodiment, the treatment composition comprises: 10-20 wt % crosslinkable microgel particles; an initiator; 0.5-2 wt % NaOH; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an initiator; 0.5-2 wt % NaOH; and is characterised by a pH between 7.2 and 7.6. In an embodiment, the treatment composition comprises: an active precursor component; ammonium persulfate; 0.5-2 wt % NaOH; and is characterised by a pH between 7.2 and 7.6. In an embodiment, the treatment composition comprises: crosslinkable microgel particles; ammonium persulfate; 0.5-2 wt % NaOH; and is characterised by a pH between 7.2 and 7.6. In an embodiment, the treatment composition comprises: 10-20 wt % crosslinkable microgel particles; ammonium persulfate; 0.5-2 wt % NaOH; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ammonium persulfate; 0.5-2 wt % NaOH; and is characterised by a pH between 7.2 and 7.6. In an embodiment, the treatment composition comprises: an active precursor component; 0.2-0.4 wt % ammonium persulfate; 0.5-2 wt % NaOH; and is characterised by a pH between 7.2 and 7.6. In an embodiment, the treatment composition comprises: crosslinkable microgel particles; 0.2-0.4 wt % ammonium persulfate; 0.5-2 wt % NaOH; and is characterised by a pH between 7.2 and 7.6. In an embodiment, the treatment composition comprises: 10-20 wt % crosslinkable microgel particles; 0.2-0.4 wt % ammonium persulfate; 0.5-2 wt % NaOH; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.2-0.4 wt % ammonium persulfate; 0.5-2 wt % NaOH; and is characterised by a pH between 7.2 and 7.6. In an embodiment, the treatment composition comprises: an active precursor component; an accelerator; an initiator; a pH modifier; and is characterised by a pH between 6.5 and 8. In an embodiment, the treatment composition comprises: crosslinkable microgel particles; an accelerator; an initiator; a pH modifier; and is characterised by a pH between 6.5 and 8. In an embodiment, the treatment composition comprises: 10-20 wt % crosslinkable microgel particles; an accelerator; an initiator; a pH modifier; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; an initiator; a pH modifier; and is characterised by a pH between 6.5 and 8. In an embodiment, the treatment composition comprises: an active precursor component; ascorbic acid (or a salt thereof); an initiator; a pH modifier; and is characterised by a pH between 6.5 and 8. In an embodiment, the treatment composition comprises: crosslinkable microgel particles; ascorbic acid (or a salt thereof); an initiator; a pH modifier; and is characterised by a pH between 6.5 and 8. In an embodiment, the treatment composition comprises: 10-20 wt % crosslinkable microgel particles; ascorbic acid (or a salt thereof); an initiator; a pH modifier; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); an initiator; a pH modifier; and is characterised by a pH between 6.5 and 8. In an embodiment, the treatment composition comprises: an active precursor component; 0.05-0.15 wt % ascorbic acid (or a salt thereof); an initiator; a pH modifier; and is characterised by a pH between 6.5 and 8. In an embodiment, the treatment composition comprises: crosslinkable microgel particles; 0.05-0.15 wt % ascorbic acid (or a salt thereof); an initiator; a pH modifier; and is characterised by a pH between 6.5 and 8. In an embodiment, the treatment composition comprises: 10-20 wt % crosslinkable microgel particles; 0.05-0.15 wt % ascorbic acid (or a salt thereof); an initiator; a pH modifier; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); an initiator; a pH modifier; and is characterised by a pH between 6.5 and 8. In an embodiment, the treatment composition comprises: an active precursor component; an accelerator; ammonium persulfate; a pH modifier; and is characterised by a pH between 6.5 and 8. In an embodiment, the treatment composition comprises: crosslinkable microgel particles; an accelerator; ammonium persulfate; a pH modifier; and is characterised by a pH between 6.5 and 8. In an embodiment, the treatment composition comprises: 10-20 wt % crosslinkable microgel particles; an accelerator; ammonium persulfate; a pH modifier; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; ammonium persulfate; a pH modifier; and is characterised by a pH between 6.5 and 8. In an embodiment, the treatment composition comprises: an active precursor component; ascorbic acid (or a salt thereof); ammonium persulfate; a pH modifier; and is characterised by a pH between 6.5 and 8. In an embodiment, the treatment composition comprises: crosslinkable microgel particles; ascorbic acid (or a salt thereof); ammonium persulfate; a pH modifier; and is characterised by a pH between 6.5 and 8. In an embodiment, the treatment composition comprises: 10-20 wt % crosslinkable microgel particles; ascorbic acid (or a salt thereof); ammonium persulfate; a pH modifier; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); ammonium persulfate; a pH modifier; and is characterised by a pH between 6.5 and 8. In an embodiment, the treatment composition comprises: an active precursor component; 0.05-0.15 wt % ascorbic acid (or a salt thereof); ammonium persulfate; a pH modifier; and is characterised by a pH between 6.5 and 8. In an embodiment, the treatment composition comprises: crosslinkable microgel particles; 0.05-0.15 wt % ascorbic acid (or a salt thereof); ammonium persulfate; a pH modifier; and is characterised by a pH between 6.5 and 8. In an embodiment, the treatment composition comprises: 10-20 wt % crosslinkable microgel particles; 0.05-0.15 wt % ascorbic acid (or a salt thereof); ammonium persulfate; a pH modifier; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); ammonium persulfate; a pH modifier; and is characterised by a pH between 6.5 and 8. In an embodiment, the treatment composition comprises: an active precursor component; an accelerator; 0.2-0.4 wt % ammonium persulfate; a pH modifier; and is characterised by a pH between 6.5 and 8. In an embodiment, the treatment composition comprises: crosslinkable microgel particles; an accelerator; 0.2-0.4 wt % ammonium persulfate; a pH modifier; and is characterised by a pH between 6.5 and 8. In an embodiment, the treatment composition comprises: 10-20 wt % crosslinkable microgel particles; an accelerator; 0.2-0.4 wt % ammonium persulfate; a pH modifier; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; 0.2-0.4 wt % ammonium persulfate; a pH modifier; and is characterised by a pH between 6.5 and 8. In an embodiment, the treatment composition comprises: an active precursor component; ascorbic acid (or a salt thereof); 0.2-0.4 wt % ammonium persulfate; a pH modifier; and is characterised by a pH between 6.5 and 8. In an embodiment, the treatment composition comprises: crosslinkable microgel particles; ascorbic acid (or a salt thereof); 0.2-0.4 wt % ammonium persulfate; a pH modifier; and is characterised by a pH between 6.5 and 8. In an embodiment, the treatment composition comprises: 10-20 wt % crosslinkable microgel particles; ascorbic acid (or a salt thereof); 0.2-0.4 wt % ammonium persulfate; a pH modifier; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); 0.2-0.4 wt % ammonium persulfate; a pH modifier; and is characterised by a pH between 6.5 and 8. In an embodiment, the treatment composition comprises: an active precursor component; 0.05-0.15 wt % ascorbic acid (or a salt thereof); 0.2-0.4 wt % ammonium persulfate; a pH modifier; and is characterised by a pH between 6.5 and 8. In an embodiment, the treatment composition comprises: crosslinkable microgel particles; 0.05-0.15 wt % ascorbic acid (or a salt thereof); 0.2-0.4 wt % ammonium persulfate; a pH modifier; and is characterised by a pH between 6.5 and 8. In an embodiment, the treatment composition comprises: 10-20 wt % crosslinkable microgel particles; 0.05-0.15 wt % ascorbic acid (or a salt thereof); 0.2-0.4 wt % ammonium persulfate; a pH modifier; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); 0.2-0.4 wt % ammonium persulfate; a pH modifier; and is characterised by a pH between 6.5 and 8. In an embodiment, the treatment composition comprises: an active precursor component; an accelerator; an initiator; sodium hydroxide; and is characterised by a pH between 6.5 and 8. In an embodiment, the treatment composition comprises: crosslinkable microgel particles; an accelerator; an initiator; sodium hydroxide; and is characterised by a pH between 6.5 and 8. In an embodiment, the treatment composition comprises: 10-20 wt % crosslinkable microgel particles; an accelerator; an initiator; sodium hydroxide; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; an initiator; sodium hydroxide; and is characterised by a pH between 6.5 and 8. In an embodiment, the treatment composition comprises: an active precursor component; ascorbic acid (or a salt thereof); an initiator; sodium hydroxide; and is characterised by a pH between 6.5 and 8. In an embodiment, the treatment composition comprises: crosslinkable microgel particles; ascorbic acid (or a salt thereof); an initiator; sodium hydroxide; and is characterised by a pH between 6.5 and 8. In an embodiment, the treatment composition comprises: 10-20 wt % crosslinkable microgel particles; ascorbic acid (or a salt thereof); an initiator; sodium hydroxide; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); an initiator; sodium hydroxide; and is characterised by a pH between 6.5 and 8. In an embodiment, the treatment composition comprises: an active precursor component; 0.05-0.15 wt % ascorbic acid (or a salt thereof); an initiator; sodium hydroxide; and is characterised by a pH between 6.5 and 8. In an embodiment, the treatment composition comprises: crosslinkable microgel particles; 0.05-0.15 wt % ascorbic acid (or a salt thereof); an initiator; sodium hydroxide; and is characterised by a pH between 6.5 and 8. In an embodiment, the treatment composition comprises: 10-20 wt % crosslinkable microgel particles; 0.05-0.15 wt % ascorbic acid (or a salt thereof); an initiator; sodium hydroxide; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); an initiator; sodium hydroxide; and is characterised by a pH between 6.5 and 8. In an embodiment, the treatment composition comprises: an active precursor component; an accelerator; ammonium persulfate; sodium hydroxide; and is characterised by a pH between 6.5 and 8. In an embodiment, the treatment composition comprises: crosslinkable microgel particles; an accelerator; ammonium persulfate; sodium hydroxide; and is characterised by a pH between 6.5 and 8. In an embodiment, the treatment composition comprises: 10-20 wt % crosslinkable microgel particles; an accelerator; ammonium persulfate; sodium hydroxide; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; ammonium persulfate; sodium hydroxide; and is characterised by a pH between 6.5 and 8. In an embodiment, the treatment composition comprises: an active precursor component; ascorbic acid (or a salt thereof); ammonium persulfate; sodium hydroxide; and is characterised by a pH between 6.5 and 8. In an embodiment, the treatment composition comprises: crosslinkable microgel particles; ascorbic acid (or a salt thereof); ammonium persulfate; sodium hydroxide; and is characterised by a pH between 6.5 and 8. In an embodiment, the treatment composition comprises: 10-20 wt % crosslinkable microgel particles; ascorbic acid (or a salt thereof); ammonium persulfate; sodium hydroxide; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); ammonium persulfate; sodium hydroxide; and is characterised by a pH between 6.5 and 8. In an embodiment, the treatment composition comprises: an active precursor component; 0.05-0.15 wt % ascorbic acid (or a salt thereof); ammonium persulfate; sodium hydroxide; and is characterised by a pH between 6.5 and 8. In an embodiment, the treatment composition comprises: crosslinkable microgel particles; 0.05-0.15 wt % ascorbic acid (or a salt thereof); ammonium persulfate; sodium hydroxide; and is characterised by a pH between 6.5 and 8. In an embodiment, the treatment composition comprises: 10-20 wt % crosslinkable microgel particles; 0.05-0.15 wt % ascorbic acid (or a salt thereof); ammonium persulfate; sodium hydroxide; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); ammonium persulfate; sodium hydroxide; and is characterised by a pH between 6.5 and 8. In an embodiment, the treatment composition comprises: an active precursor component; an accelerator; 0.2-0.4 wt % ammonium persulfate; sodium hydroxide; and is characterised by a pH between 6.5 and 8. In an embodiment, the treatment composition comprises: crosslinkable microgel particles; an accelerator; 0.2-0.4 wt % ammonium persulfate; sodium hydroxide; and is characterised by a pH between 6.5 and 8. In an embodiment, the treatment composition comprises: 10-20 wt % crosslinkable microgel particles; an accelerator; 0.2-0.4 wt % ammonium persulfate; sodium hydroxide; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; 0.2-0.4 wt % ammonium persulfate; sodium hydroxide; and is characterised by a pH between 6.5 and 8. In an embodiment, the treatment composition comprises: an active precursor component; ascorbic acid (or a salt thereof); 0.2-0.4 wt % ammonium persulfate; sodium hydroxide; and is characterised by a pH between 6.5 and 8. In an embodiment, the treatment composition comprises: crosslinkable microgel particles; ascorbic acid (or a salt thereof); 0.2-0.4 wt % ammonium persulfate; sodium hydroxide; and is characterised by a pH between 6.5 and 8. In an embodiment, the treatment composition comprises: 10-20 wt % crosslinkable microgel particles; ascorbic acid (or a salt thereof); 0.2-0.4 wt % ammonium persulfate; sodium hydroxide; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); 0.2-0.4 wt % ammonium persulfate; sodium hydroxide; and is characterised by a pH between 6.5 and 8. In an embodiment, the treatment composition comprises: an active precursor component; 0.05-0.15 wt % ascorbic acid (or a salt thereof); 0.2-0.4 wt % ammonium persulfate; sodium hydroxide; and is characterised by a pH between 6.5 and 8. In an embodiment, the treatment composition comprises: crosslinkable microgel particles; 0.05-0.15 wt % ascorbic acid (or a salt thereof); 0.2-0.4 wt % ammonium persulfate; sodium hydroxide; and is characterised by a pH between 6.5 and 8. In an embodiment, the treatment composition comprises: 10-20 wt % crosslinkable microgel particles; 0.05-0.15 wt % ascorbic acid (or a salt thereof); 0.2-0.4 wt % ammonium persulfate; sodium hydroxide; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); 0.2-0.4 wt % ammonium persulfate; sodium hydroxide; and is characterised by a pH between 6.5 and 8. In an embodiment, the treatment composition comprises: an active precursor component; an accelerator; an initiator; 0.5-2 wt % NaOH; and is characterised by a pH between 6.5 and 8. In an embodiment, the treatment composition comprises: crosslinkable microgel particles; an accelerator; an initiator; 0.5-2 wt % NaOH; and is characterised by a pH between 6.5 and 8. In an embodiment, the treatment composition comprises: 10-20 wt % crosslinkable microgel particles; an accelerator; an initiator; 0.5-2 wt % NaOH; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; an initiator; 0.5-2 wt % NaOH; and is characterised by a pH between 6.5 and 8. In an embodiment, the treatment composition comprises: an active precursor component; ascorbic acid (or a salt thereof); an initiator; 0.5-2 wt % NaOH; and is characterised by a pH between 6.5 and 8. In an embodiment, the treatment composition comprises: crosslinkable microgel particles; ascorbic acid (or a salt thereof); an initiator; 0.5-2 wt % NaOH; and is characterised by a pH between 6.5 and 8. In an embodiment, the treatment composition comprises: 10-20 wt % crosslinkable microgel particles; ascorbic acid (or a salt thereof); an initiator; 0.5-2 wt % NaOH; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); an initiator; 0.5-2 wt % NaOH; and is characterised by a pH between 6.5 and 8. In an embodiment, the treatment composition comprises: an active precursor component; 0.05-0.15 wt % ascorbic acid (or a salt thereof); an initiator; 0.5-2 wt % NaOH; and is characterised by a pH between 6.5 and 8. In an embodiment, the treatment composition comprises: crosslinkable microgel particles; 0.05-0.15 wt % ascorbic acid (or a salt thereof); an initiator; 0.5-2 wt % NaOH; and is characterised by a pH between 6.5 and 8. In an embodiment, the treatment composition comprises: 10-20 wt % crosslinkable microgel particles; 0.05-0.15 wt % ascorbic acid (or a salt thereof); an initiator; 0.5-2 wt % NaOH; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); an initiator; 0.5-2 wt % NaOH; and is characterised by a pH between 6.5 and 8. In an embodiment, the treatment composition comprises: an active precursor component; an accelerator; ammonium persulfate; 0.5-2 wt % NaOH; and is characterised by a pH between 6.5 and 8. In an embodiment, the treatment composition comprises: crosslinkable microgel particles; an accelerator; ammonium persulfate; 0.5-2 wt % NaOH; and is characterised by a pH between 6.5 and 8. In an embodiment, the treatment composition comprises: 10-20 wt % crosslinkable microgel particles; an accelerator; ammonium persulfate; 0.5-2 wt % NaOH; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; ammonium persulfate; 0.5-2 wt % NaOH; and is characterised by a pH between 6.5 and 8. In an embodiment, the treatment composition comprises: an active precursor component; ascorbic acid (or a salt thereof); ammonium persulfate; 0.5-2 wt % NaOH; and is characterised by a pH between 6.5 and 8. In an embodiment, the treatment composition comprises: crosslinkable microgel particles; ascorbic acid (or a salt thereof); ammonium persulfate; 0.5-2 wt % NaOH; and is characterised by a pH between 6.5 and 8. In an embodiment, the treatment composition comprises: 10-20 wt % crosslinkable microgel particles; ascorbic acid (or a salt thereof); ammonium persulfate; 0.5-2 wt % NaOH; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); ammonium persulfate; 0.5-2 wt % NaOH; and is characterised by a pH between 6.5 and 8. In an embodiment, the treatment composition comprises: an active precursor component; 0.05-0.15 wt % ascorbic acid (or a salt thereof); ammonium persulfate; 0.5-2 wt % NaOH; and is characterised by a pH between 6.5 and 8. In an embodiment, the treatment composition comprises: crosslinkable microgel particles; 0.05-0.15 wt % ascorbic acid (or a salt thereof); ammonium persulfate; 0.5-2 wt % NaOH; and is characterised by a pH between 6.5 and 8. In an embodiment, the treatment composition comprises: 10-20 wt % crosslinkable microgel particles; 0.05-0.15 wt % ascorbic acid (or a salt thereof); ammonium persulfate; 0.5-2 wt % NaOH; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); ammonium persulfate; 0.5-2 wt % NaOH; and is characterised by a pH between 6.5 and 8. In an embodiment, the treatment composition comprises: an active precursor component; an accelerator; 0.2-0.4 wt % ammonium persulfate; 0.5-2 wt % NaOH; and is characterised by a pH between 6.5 and 8. In an embodiment, the treatment composition comprises: crosslinkable microgel particles; an accelerator; 0.2-0.4 wt % ammonium persulfate; 0.5-2 wt % NaOH; and is characterised by a pH between 6.5 and 8. In an embodiment, the treatment composition comprises: 10-20 wt % crosslinkable microgel particles; an accelerator; 0.2-0.4 wt % ammonium persulfate; 0.5-2 wt % NaOH; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; 0.2-0.4 wt % ammonium persulfate; 0.5-2 wt % NaOH; and is characterised by a pH between 6.5 and 8. In an embodiment, the treatment composition comprises: an active precursor component; ascorbic acid (or a salt thereof); 0.2-0.4 wt % ammonium persulfate; 0.5-2 wt % NaOH; and is characterised by a pH between 6.5 and 8. In an embodiment, the treatment composition comprises: crosslinkable microgel particles; ascorbic acid (or a salt thereof); 0.2-0.4 wt % ammonium persulfate; 0.5-2 wt % NaOH; and is characterised by a pH between 6.5 and 8. In an embodiment, the treatment composition comprises: 10-20 wt % crosslinkable microgel particles; ascorbic acid (or a salt thereof); 0.2-0.4 wt % ammonium persulfate; 0.5-2 wt % NaOH; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); 0.2-0.4 wt % ammonium persulfate; 0.5-2 wt % NaOH; and is characterised by a pH between 6.5 and 8. In an embodiment, the treatment composition comprises: an active precursor component; 0.05-0.15 wt % ascorbic acid (or a salt thereof); 0.2-0.4 wt % ammonium persulfate; 0.5-2 wt % NaOH; and is characterised by a pH between 6.5 and 8. In an embodiment, the treatment composition comprises: crosslinkable microgel particles; 0.05-0.15 wt % ascorbic acid (or a salt thereof); 0.2-0.4 wt % ammonium persulfate; 0.5-2 wt % NaOH; and is characterised by a pH between 6.5 and 8. In an embodiment, the treatment composition comprises: 10-20 wt % crosslinkable microgel particles; 0.05-0.15 wt % ascorbic acid (or a salt thereof); 0.2-0.4 wt % ammonium persulfate; 0.5-2 wt % NaOH; and is characterised by a pH between 6.5 and 8. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); 0.2-0.4 wt % ammonium persulfate; 0.5-2 wt % NaOH; and is characterised by a pH between 6.5 and 8. In an embodiment, the treatment composition comprises: an active precursor component; an accelerator; an initiator; a pH modifier; and is characterised by a pH between 7.2 and 7.6. In an embodiment, the treatment composition comprises: crosslinkable microgel particles; an accelerator; an initiator; a pH modifier; and is characterised by a pH between 7.2 and 7.6. In an embodiment, the treatment composition comprises: 10-20 wt % crosslinkable microgel particles; an accelerator; an initiator; a pH modifier; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; an initiator; a pH modifier; and is characterised by a pH between 7.2 and 7.6. In an embodiment, the treatment composition comprises: an active precursor component; ascorbic acid (or a salt thereof); an initiator; a pH modifier; and is characterised by a pH between 7.2 and 7.6. In an embodiment, the treatment composition comprises: crosslinkable microgel particles; ascorbic acid (or a salt thereof); an initiator; a pH modifier; and is characterised by a pH between 7.2 and 7.6. In an embodiment, the treatment composition comprises: 10-20 wt % crosslinkable microgel particles; ascorbic acid (or a salt thereof); an initiator; a pH modifier; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); an initiator; a pH modifier; and is characterised by a pH between 7.2 and 7.6. In an embodiment, the treatment composition comprises: an active precursor component; 0.05-0.15 wt % ascorbic acid (or a salt thereof); an initiator; a pH modifier; and is characterised by a pH between 7.2 and 7.6. In an embodiment, the treatment composition comprises: crosslinkable microgel particles; 0.05-0.15 wt % ascorbic acid (or a salt thereof); an initiator; a pH modifier; and is characterised by a pH between 7.2 and 7.6. In an embodiment, the treatment composition comprises: 10-20 wt % crosslinkable microgel particles; 0.05-0.15 wt % ascorbic acid (or a salt thereof); an initiator; a pH modifier; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); an initiator; a pH modifier; and is characterised by a pH between 7.2 and 7.6. In an embodiment, the treatment composition comprises: an active precursor component; an accelerator; ammonium persulfate; a pH modifier; and is characterised by a pH between 7.2 and 7.6. In an embodiment, the treatment composition comprises: crosslinkable microgel particles; an accelerator; ammonium persulfate; a pH modifier; and is characterised by a pH between 7.2 and 7.6. In an embodiment, the treatment composition comprises: 10-20 wt % crosslinkable microgel particles; an accelerator; ammonium persulfate; a pH modifier; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; ammonium persulfate; a pH modifier; and is characterised by a pH between 7.2 and 7.6. In an embodiment, the treatment composition comprises: an active precursor component; ascorbic acid (or a salt thereof); ammonium persulfate; a pH modifier; and is characterised by a pH between 7.2 and 7.6. In an embodiment, the treatment composition comprises: crosslinkable microgel particles; ascorbic acid (or a salt thereof); ammonium persulfate; a pH modifier; and is characterised by a pH between 7.2 and 7.6. In an embodiment, the treatment composition comprises: 10-20 wt % crosslinkable microgel particles; ascorbic acid (or a salt thereof); ammonium persulfate; a pH modifier; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); ammonium persulfate; a pH modifier; and is characterised by a pH between 7.2 and 7.6. In an embodiment, the treatment composition comprises: an active precursor component; 0.05-0.15 wt % ascorbic acid (or a salt thereof); ammonium persulfate; a pH modifier; and is characterised by a pH between 7.2 and 7.6. In an embodiment, the treatment composition comprises: crosslinkable microgel particles; 0.05-0.15 wt % ascorbic acid (or a salt thereof); ammonium persulfate; a pH modifier; and is characterised by a pH between 7.2 and 7.6. In an embodiment, the treatment composition comprises: 10-20 wt % crosslinkable microgel particles; 0.05-0.15 wt % ascorbic acid (or a salt thereof); ammonium persulfate; a pH modifier; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); ammonium persulfate; a pH modifier; and is characterised by a pH between 7.2 and 7.6. In an embodiment, the treatment composition comprises: an active precursor component; an accelerator; 0.2-0.4 wt % ammonium persulfate; a pH modifier; and is characterised by a pH between 7.2 and 7.6. In an embodiment, the treatment composition comprises: crosslinkable microgel particles; an accelerator; 0.2-0.4 wt % ammonium persulfate; a pH modifier; and is characterised by a pH between 7.2 and 7.6. In an embodiment, the treatment composition comprises: 10-20 wt % crosslinkable microgel particles; an accelerator; 0.2-0.4 wt % ammonium persulfate; a pH modifier; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; 0.2-0.4 wt % ammonium persulfate; a pH modifier; and is characterised by a pH between 7.2 and 7.6. In an embodiment, the treatment composition comprises: an active precursor component; ascorbic acid (or a salt thereof); 0.2-0.4 wt % ammonium persulfate; a pH modifier; and is characterised by a pH between 7.2 and 7.6. In an embodiment, the treatment composition comprises: crosslinkable microgel particles; ascorbic acid (or a salt thereof); 0.2-0.4 wt % ammonium persulfate; a pH modifier; and is characterised by a pH between 7.2 and 7.6. In an embodiment, the treatment composition comprises: 10-20 wt % crosslinkable microgel particles; ascorbic acid (or a salt thereof); 0.2-0.4 wt % ammonium persulfate; a pH modifier; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); 0.2-0.4 wt % ammonium persulfate; a pH modifier; and is characterised by a pH between 7.2 and 7.6. In an embodiment, the treatment composition comprises: an active precursor component; 0.05-0.15 wt % ascorbic acid (or a salt thereof); 0.2-0.4 wt % ammonium persulfate; a pH modifier; and is characterised by a pH between 7.2 and 7.6. In an embodiment, the treatment composition comprises: crosslinkable microgel particles; 0.05-0.15 wt % ascorbic acid (or a salt thereof); 0.2-0.4 wt % ammonium persulfate; a pH modifier; and is characterised by a pH between 7.2 and 7.6. In an embodiment, the treatment composition comprises: 10-20 wt % crosslinkable microgel particles; 0.05-0.15 wt % ascorbic acid (or a salt thereof); 0.2-0.4 wt % ammonium persulfate; a pH modifier; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); 0.2-0.4 wt % ammonium persulfate; a pH modifier; and is characterised by a pH between 7.2 and 7.6. In an embodiment, the treatment composition comprises: an active precursor component; an accelerator; an initiator; sodium hydroxide; and is characterised by a pH between 7.2 and 7.6. In an embodiment, the treatment composition comprises: crosslinkable microgel particles; an accelerator; an initiator; sodium hydroxide; and is characterised by a pH between 7.2 and 7.6. In an embodiment, the treatment composition comprises: 10-20 wt % crosslinkable microgel particles; an accelerator; an initiator; sodium hydroxide; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; an initiator; sodium hydroxide; and is characterised by a pH between 7.2 and 7.6. In an embodiment, the treatment composition comprises: an active precursor component; ascorbic acid (or a salt thereof); an initiator; sodium hydroxide; and is characterised by a pH between 7.2 and 7.6. In an embodiment, the treatment composition comprises: crosslinkable microgel particles; ascorbic acid (or a salt thereof); an initiator; sodium hydroxide; and is characterised by a pH between 7.2 and 7.6. In an embodiment, the treatment composition comprises: 10-20 wt % crosslinkable microgel particles; ascorbic acid (or a salt thereof); an initiator; sodium hydroxide; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); an initiator; sodium hydroxide; and is characterised by a pH between 7.2 and 7.6. In an embodiment, the treatment composition comprises: an active precursor component; 0.05-0.15 wt % ascorbic acid (or a salt thereof); an initiator; sodium hydroxide; and is characterised by a pH between 7.2 and 7.6. In an embodiment, the treatment composition comprises: crosslinkable microgel particles; 0.05-0.15 wt % ascorbic acid (or a salt thereof); an initiator; sodium hydroxide; and is characterised by a pH between 7.2 and 7.6. In an embodiment, the treatment composition comprises: 10-20 wt % crosslinkable microgel particles; 0.05-0.15 wt % ascorbic acid (or a salt thereof); an initiator; sodium hydroxide; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); an initiator; sodium hydroxide; and is characterised by a pH between 7.2 and 7.6. In an embodiment, the treatment composition comprises: an active precursor component; an accelerator; ammonium persulfate; sodium hydroxide; and is characterised by a pH between 7.2 and 7.6. In an embodiment, the treatment composition comprises: crosslinkable microgel particles; an accelerator; ammonium persulfate; sodium hydroxide; and is characterised by a pH between 7.2 and 7.6. In an embodiment, the treatment composition comprises: 10-20 wt % crosslinkable microgel particles; an accelerator; ammonium persulfate; sodium hydroxide; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; ammonium persulfate; sodium hydroxide; and is characterised by a pH between 7.2 and 7.6. In an embodiment, the treatment composition comprises: an active precursor component; ascorbic acid (or a salt thereof); ammonium persulfate; sodium hydroxide; and is characterised by a pH between 7.2 and 7.6. In an embodiment, the treatment composition comprises: crosslinkable microgel particles; ascorbic acid (or a salt thereof); ammonium persulfate; sodium hydroxide; and is characterised by a pH between 7.2 and 7.6. In an embodiment, the treatment composition comprises: 10-20 wt % crosslinkable microgel particles; ascorbic acid (or a salt thereof); ammonium persulfate; sodium hydroxide; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); ammonium persulfate; sodium hydroxide; and is characterised by a pH between 7.2 and 7.6. In an embodiment, the treatment composition comprises: an active precursor component; 0.05-0.15 wt % ascorbic acid (or a salt thereof); ammonium persulfate; sodium hydroxide; and is characterised by a pH between 7.2 and 7.6. In an embodiment, the treatment composition comprises: crosslinkable microgel particles; 0.05-0.15 wt % ascorbic acid (or a salt thereof); ammonium persulfate; sodium hydroxide; and is characterised by a pH between 7.2 and 7.6. In an embodiment, the treatment composition comprises: 10-20 wt % crosslinkable microgel particles; 0.05-0.15 wt % ascorbic acid (or a salt thereof); ammonium persulfate; sodium hydroxide; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); ammonium persulfate; sodium hydroxide; and is characterised by a pH between 7.2 and 7.6. In an embodiment, the treatment composition comprises: an active precursor component; an accelerator; 0.2-0.4 wt % ammonium persulfate; sodium hydroxide; and is characterised by a pH between 7.2 and 7.6. In an embodiment, the treatment composition comprises: crosslinkable microgel particles; an accelerator; 0.2-0.4 wt % ammonium persulfate; sodium hydroxide; and is characterised by a pH between 7.2 and 7.6. In an embodiment, the treatment composition comprises: 10-20 wt % crosslinkable microgel particles; an accelerator; 0.2-0.4 wt % ammonium persulfate; sodium hydroxide; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; 0.2-0.4 wt % ammonium persulfate; sodium hydroxide; and is characterised by a pH between 7.2 and 7.6. In an embodiment, the treatment composition comprises: an active precursor component; ascorbic acid (or a salt thereof); 0.2-0.4 wt % ammonium persulfate; sodium hydroxide; and is characterised by a pH between 7.2 and 7.6. In an embodiment, the treatment composition comprises: crosslinkable microgel particles; ascorbic acid (or a salt thereof); 0.2-0.4 wt % ammonium persulfate; sodium hydroxide; and is characterised by a pH between 7.2 and 7.6. In an embodiment, the treatment composition comprises: 10-20 wt % crosslinkable microgel particles; ascorbic acid (or a salt thereof); 0.2-0.4 wt % ammonium persulfate; sodium hydroxide; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); 0.2-0.4 wt % ammonium persulfate; sodium hydroxide; and is characterised by a pH between 7.2 and 7.6. In an embodiment, the treatment composition comprises: an active precursor component; 0.05-0.15 wt % ascorbic acid (or a salt thereof); 0.2-0.4 wt % ammonium persulfate; sodium hydroxide; and is characterised by a pH between 7.2 and 7.6. In an embodiment, the treatment composition comprises: crosslinkable microgel particles; 0.05-0.15 wt % ascorbic acid (or a salt thereof); 0.2-0.4 wt % ammonium persulfate; sodium hydroxide; and is characterised by a pH between 7.2 and 7.6. In an embodiment, the treatment composition comprises: 10-20 wt % crosslinkable microgel particles; 0.05-0.15 wt % ascorbic acid (or a salt thereof); 0.2-0.4 wt % ammonium persulfate; sodium hydroxide; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); 0.2-0.4 wt % ammonium persulfate; sodium hydroxide; and is characterised by a pH between 7.2 and 7.6. In an embodiment, the treatment composition comprises: an active precursor component; an accelerator; an initiator; 0.5-2 wt % NaOH; and is characterised by a pH between 7.2 and 7.6. In an embodiment, the treatment composition comprises: crosslinkable microgel particles; an accelerator; an initiator; 0.5-2 wt % NaOH; and is characterised by a pH between 7.2 and 7.6. In an embodiment, the treatment composition comprises: 10-20 wt % crosslinkable microgel particles; an accelerator; an initiator; 0.5-2 wt % NaOH; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; an initiator; 0.5-2 wt % NaOH; and is characterised by a pH between 7.2 and 7.6. In an embodiment, the treatment composition comprises: an active precursor component; ascorbic acid (or a salt thereof); an initiator; 0.5-2 wt % NaOH; and is characterised by a pH between 7.2 and 7.6. In an embodiment, the treatment composition comprises: crosslinkable microgel particles; ascorbic acid (or a salt thereof); an initiator; 0.5-2 wt % NaOH; and is characterised by a pH between 7.2 and 7.6. In an embodiment, the treatment composition comprises: 10-20 wt % crosslinkable microgel particles; ascorbic acid (or a salt thereof); an initiator; 0.5-2 wt % NaOH; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); an initiator; 0.5-2 wt % NaOH; and is characterised by a pH between 7.2 and 7.6. In an embodiment, the treatment composition comprises: an active precursor component; 0.05-0.15 wt % ascorbic acid (or a salt thereof); an initiator; 0.5-2 wt % NaOH; and is characterised by a pH between 7.2 and 7.6. In an embodiment, the treatment composition comprises: crosslinkable microgel particles; 0.05-0.15 wt % ascorbic acid (or a salt thereof); an initiator; 0.5-2 wt % NaOH; and is characterised by a pH between 7.2 and 7.6. In an embodiment, the treatment composition comprises: 10-20 wt % crosslinkable microgel particles; 0.05-0.15 wt % ascorbic acid (or a salt thereof); an initiator; 0.5-2 wt % NaOH; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); an initiator; 0.5-2 wt % NaOH; and is characterised by a pH between 7.2 and 7.6. In an embodiment, the treatment composition comprises: an active precursor component; an accelerator; ammonium persulfate; 0.5-2 wt % NaOH; and is characterised by a pH between 7.2 and 7.6. In an embodiment, the treatment composition comprises: crosslinkable microgel particles; an accelerator; ammonium persulfate; 0.5-2 wt % NaOH; and is characterised by a pH between 7.2 and 7.6. In an embodiment, the treatment composition comprises: 10-20 wt % crosslinkable microgel particles; an accelerator; ammonium persulfate; 0.5-2 wt % NaOH; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; ammonium persulfate; 0.5-2 wt % NaOH; and is characterised by a pH between 7.2 and 7.6. In an embodiment, the treatment composition comprises: an active precursor component; ascorbic acid (or a salt thereof); ammonium persulfate; 0.5-2 wt % NaOH; and is characterised by a pH between 7.2 and 7.6. In an embodiment, the treatment composition comprises: crosslinkable microgel particles; ascorbic acid (or a salt thereof); ammonium persulfate; 0.5-2 wt % NaOH; and is characterised by a pH between 7.2 and 7.6. In an embodiment, the treatment composition comprises: 10-20 wt % crosslinkable microgel particles; ascorbic acid (or a salt thereof); ammonium persulfate; 0.5-2 wt % NaOH; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); ammonium persulfate; 0.5-2 wt % NaOH; and is characterised by a pH between 7.2 and 7.6. In an embodiment, the treatment composition comprises: an active precursor component; 0.05-0.15 wt % ascorbic acid (or a salt thereof); ammonium persulfate; 0.5-2 wt % NaOH; and is characterised by a pH between 7.2 and 7.6. In an embodiment, the treatment composition comprises: crosslinkable microgel particles; 0.05-0.15 wt % ascorbic acid (or a salt thereof); ammonium persulfate; 0.5-2 wt % NaOH; and is characterised by a pH between 7.2 and 7.6. In an embodiment, the treatment composition comprises: 10-20 wt % crosslinkable microgel particles; 0.05-0.15 wt % ascorbic acid (or a salt thereof); ammonium persulfate; 0.5-2 wt % NaOH; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); ammonium persulfate; 0.5-2 wt % NaOH; and is characterised by a pH between 7.2 and 7.6. In an embodiment, the treatment composition comprises: an active precursor component; an accelerator; 0.2-0.4 wt % ammonium persulfate; 0.5-2 wt % NaOH; and is characterised by a pH between 7.2 and 7.6. In an embodiment, the treatment composition comprises: crosslinkable microgel particles; an accelerator; 0.2-0.4 wt % ammonium persulfate; 0.5-2 wt % NaOH; and is characterised by a pH between 7.2 and 7.6. In an embodiment, the treatment composition comprises: 10-20 wt % crosslinkable microgel particles; an accelerator; 0.2-0.4 wt % ammonium persulfate; 0.5-2 wt % NaOH; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; an accelerator; 0.2-0.4 wt % ammonium persulfate; 0.5-2 wt % NaOH; and is characterised by a pH between 7.2 and 7.6. In an embodiment, the treatment composition comprises: an active precursor component; ascorbic acid (or a salt thereof); 0.2-0.4 wt % ammonium persulfate; 0.5-2 wt % NaOH; and is characterised by a pH between 7.2 and 7.6. In an embodiment, the treatment composition comprises: crosslinkable microgel particles; ascorbic acid (or a salt thereof); 0.2-0.4 wt % ammonium persulfate; 0.5-2 wt % NaOH; and is characterised by a pH between 7.2 and 7.6. In an embodiment, the treatment composition comprises: 10-20 wt % crosslinkable microgel particles; ascorbic acid (or a salt thereof); 0.2-0.4 wt % ammonium persulfate; 0.5-2 wt % NaOH; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; ascorbic acid (or a salt thereof); 0.2-0.4 wt % ammonium persulfate; 0.5-2 wt % NaOH; and is characterised by a pH between 7.2 and 7.6. In an embodiment, the treatment composition comprises: an active precursor component; 0.05-0.15 wt % ascorbic acid (or a salt thereof); 0.2-0.4 wt % ammonium persulfate; 0.5-2 wt % NaOH; and is characterised by a pH between 7.2 and 7.6. In an embodiment, the treatment composition comprises: crosslinkable microgel particles; 0.05-0.15 wt % ascorbic acid (or a salt thereof); 0.2-0.4 wt % ammonium persulfate; 0.5-2 wt % NaOH; and is characterised by a pH between 7.2 and 7.6. In an embodiment, the treatment composition comprises: 10-20 wt % crosslinkable microgel particles; 0.05-0.15 wt % ascorbic acid (or a salt thereof); 0.2-0.4 wt % ammonium persulfate; 0.5-2 wt % NaOH; and is characterised by a pH between 7.2 and 7.6. In an embodiment, the treatment composition comprises: 10-20 wt % crosslinkable microgel particles comprising poly(MMA/MAA/EGDMA) cores surface-functionalised with GMA, wherein the cores comprise about 60-70% MMA, about 30-40% MAA and 0.1 to 1% EGDMA based on the total monomer mass, and wherein 2-8 mol. % of all monomers of the cores are functionalised with GMA; 0.05-0.15 wt % ascorbic acid (or a salt thereof); 0.2-0.4 wt % ammonium persulfate; 0.5-2 wt % NaOH; and is characterised by a pH between 7.2 and 7.6.

The post-treatment composition is suitably the same as the treatment composition, except that the active precursor component is an active component. Where the active precursor component of the treatment composition is crosslinkable microgel particles, corresponding active component of the post-treatment composition is suitably crosslinked crosslinkable microgel particles, suitably wherein the microgel particles are (suitably directly) interlinked via crosslinkable moieties, suitably crosslinked via free radical reactions between said crosslinkable moieties. The post-treatment composition suitably forms in situ at the target location.

All of the aforesaid compositions, kits, and delivery devices are compatible with methods of the invention as defined herein.

Advantages of the novel compositions and kits of the invention include more facile filtration (including sterilization filtration) of pertinent compositions in preparing a composition for administration. Such compositions have also been developed to optimise mixing and reaction kinetics during and following injection.

### Device

The present invention provides a treatment composition deliver device. The delivery device is suitably an injection device for delivering a treatment composition of the invention to a target site of a candidate subject. The injection device suitably comprises two reservoirs, an activatable reservoir for containing an activatable composition as defined herein, and an activator reservoir for containing an activator composition as defined herein, wherein the two reservoirs maintain separation between the activatable and activator compositions. The injection device suitably comprises a mixing chamber. Suitably the injection device is operable to deliver the contents of both the activatable reservoir (i.e. activatable composition) and the activator reservoir (i.e. activator composition) into the mixing chamber. Suitably the injection device is operable to deliver the activatable composition and activator composition into the mixing chamber to thereby mix to form a treatment composition. Suitably the mixing chamber comprises an outlet. Suitably the mixing chamber is operable to dispense the treatment composition (formed in the mixing chamber) from the outlet. The outlet is suitably connectable/attachable to a needle or cannula, suitably a narrow-bore needle as defined elsewhere herein. Suitably the same operation to deliver the activatable composition and activator composition into the mixing chamber causes mixing and dispensing of the treatment composition from the outlet of the mixing chamber.

The injection device suitably comprises a double-barreled syringe (or dual syringe). Suitably one of the barrels or one of the syringes is an activatable barrel/syringe, suitably configured for containing an activatable composition as defined herein. Suitably the other of the barrels or other of the syringes is an activator barrel/syringe, suitably configured for containing an activator composition as defined herein. Suitably the activatable barrel contains the activatable composition and the activator barrel contains the activator composition (i.e. a loaded deliver device). Suitably the volume (or weight) ratio of the activatable composition to activator composition is between 40:1 and 1:40, suitably between 20:1 and 1:1, suitably between 10:1 and 2:1, suitably between 5:1 and 3:1, suitably about 4:1. More suitably, the volume (or weight) ratio of the activatable composition to activator composition is between 40:1 and 1:1, suitably between 20:1 and 2:1, suitably between 15:1 and 5:1, suitably between 8:1 and 12:1, suitably about 10:1. Suitably the injection device is operable to simultaneously deliver both the activatable composition and the activator composition into the mixing chamber and out of the outlet of the mixing chamber, suitably via a syringe plunger, suitably a single or dual syringe plunger.

Suitably the outlet of the mixing chamber of the injection device is connected to a needle. Suitably the needle has an outlet whose largest inner dimension (or inner diameter) is less than or equal to 2 mm, suitably less than or equal to 1.6 mm, suitably less than or equal to 1.4 mm, suitably less than or equal to 1.2 mm; and suitably whose largest inner dimension (or inner diameter) is greater than or equal to 0.2 mm, suitably greater than or equal to 0.4 mm, suitably greater than or equal to 0.5 mm. Suitably, the needle corresponds to a Birmingham Gauge between G12 and G 25, suitably between G14 and G23, most suitably between G16 and G21.

### Methods of Administration

The present invention provides a method of treating a candidate subject (suitably as defined herein, and suitably identified as defined herein) exhibiting one or more partially-degraded target site(s), the method comprising introducing or injecting a treatment composition (and suitably therefore by definition the post-treatment composition) into one or more partially-degraded target site(s) of the candidate subject.

Suitably the treatment composition is transformed into a post-treatment composition following (and possibly also during) introduction to the target site(s).

The target site(s) may be any suitably target site as defined herein, though in a particular embodiment the target site(s) is/are IVD(s). Likewise, the candidate subject may be any suitable candidate subject as defined herein, though most suitably the candidate subject is one identified with early-stage DDD.

For minimal invasiveness, the treatment composition is suitably introduced into the one or more target site(s) by injection, suitably via the smallest possible injection outlet (e.g. smallest possible needle bore). Suitably this minimizes the size of any holes formed during injection into the target site, and maximizes the likelihood of the hole healing over, or at least minimizes the likelihood of the hole growing in size or allowing any leakage therefrom. Suitably the maximum dimension (or diameter) of any hole(s) formed during the injection process is less than or equal to 3 mm, suitably less than or equal to 2 mm, suitably less than or equal to 1.8 mm, suitably less than or equal to 1.5 mm.

Suitably the treatment composition is injected directly into the target site, and the post-treatment composition is suitably contained within the target site by virtue of the architecture of the target site itself—e.g. the injected treatment composition is not contained within a jacket, expandable balloon, or other such containment device.

Where the target site(s) is an IVD, most suitably the treatment composition is not introduced by open discectomy. Instead, where the target site(s) is an IVD, the treatment composition is most suitably delivered via intradiscal injection, suitably approaching the IVD on the posterolateral aspect of the corresponding annulus, suitably following a trajectory substantially the same as or substantially similar to a discography. Suitably such injection of the treatment composition is image-guided or image-supervised, and suitably the inclusion of a contrast agent or visualization agent within the treatment composition may facilitate such image-guidance.

Suitably such methods of treatment do not require prior removal of IVD material, and suitably no IVD material is removed prior to implementation of such methods. In fact, advantageously the invention may capitalize on the presence of nucleus cells still in a capacity of being revitalized, and the presence within the IVD of natural slits and tears (e.g. within the nucleus pulposus/ECM) into which the treatment composition/post-treatment composition may flow/diffuse upon injection, thereby avoiding the formation of a bolus (i.e. monolithic mass) of post-treatment composition which may not be located at the most appropriate place within the disc space, be vulnerable to migration or expulsion (e.g. may slip out of place if a candidate subject moves awkwardly). Such advantages are further enhanced by the relative fluidity of the initial treatment composition, thereby allowing it to flow into small crevices and cracks before curing/hardening into the post-treatment composition. Meanwhile, the final gelled post-treatment composition is suitably immobilized and not susceptible to migration or redispersion (e.g. in response to compressive loads), though suitably the post-treatment composition exhibits sufficient elasticity/compressibility to withstand compression forces (e.g. without undergoing physical or chemical transformation), suitably without hysteresis or creep. Moreover, as described elsewhere herein, such post-treatment compositions suitably do not degrade since, suitably the active component of the post-treatment composition is not susceptible to enzymatic degradation. Moreover the suitably gel will not lose its water content after being placed under load.

As such, where the target site is an IVD (or nucleus pulposus thereof), suitably the treatment composition is introduced into slits, crevices, cracks or tears of the nucleus pulposus. Suitably, such filling of crevices revitalizes (or at least retards degradation of) surrounding cells. Suitably such revitalization (or degradation retardation) is thought to be the result of the sensitivity of cells to their local environment, including their physical environment (especially local hydrostatic pressure and local hydration levels, which also facilitates diffusion of nutrients). As such, the post-treatment compositions of the invention may be considered to mimic nucleus pulposus material (or the ECM thereof) and thereby persuade local cells against pursuing managed degradation. However, suitably the post-treatment composition is not a prosthetic disk nucleus (PDN) as such. Suitably the post-treatment composition does not fill the disk space, and suitably constitutes less than 50% by volume of the total disk space, suitably less than 40% by volume, suitably less than 30% by volume, suitably less than 20% by volume, suitably less than 15% by volume. Suitably, introduction of the treatment/post-treatment composition restores no more than 40% of disc height, suitably no more than 30% of disc height, suitably no more than 20% disc height, suitably no more than 15% disc height, suitably no more than 10% disc height. Suitably the amount of treatment/post-treatment composition introduced into the target site(s) is a non-load-bearing quantity (i.e. not a quantity which replaces lost materials to restore load-bearing potential). Suitably the amount of treatment/post-treatment composition deliver is sufficient to support or improve the function of existing components at the target site to perform their own load-bearing function. However, in certain embodiments the post-treatment composition may provide a load-bearing or partial-load-bearing function.

Suitably no more than 5 mL treatment composition is introduced into a specific target site (i.e. ≤5 mL per target site), suitably no more than 4 mL, suitably no more than 3 mL, suitably no more than 2.5 mL, suitably no more than 2 mL. Suitably at least 0.1 mL treatment composition is introduced into a specific target site (i.e. ≥0.1 mL per target site), suitably at least 0.2 mL, suitably at least 0.5 mL, suitably at least 0.8 mL, suitably at least 1.0 mL. Suitably 0.5-2 mL of treatment composition is introduced into a specific target site, especially where the target site is an IVD, more suitably 1-2 mL.

The aforementioned features relating to methods of treatment are applicable to target sites other than IVDs, especially where said target sites can benefit from the treatment composition and post-treatment composition of the invention. This is especially the case where local cells and cellular processes can benefit (e.g. through detecting a vitalizing level of hydration that affords better diffusion). In particular, other target sites comprising an ECM, especially cartilaginous target sites, can benefit. For cartilaginous target sites, suitably the treatment composition of the invention is introduced (suitably by injection) into the ECM if the target site, especially where said ECM is shown to bear cracks, tears, crevices, or fissures. In such circumstances, suitably any cracks, tears, crevices, or fissures of the target ECM are filled with the treatment composition before it completely cures to form a post-treatment composition.

Suitably, a treatment composition of the invention is introduced (suitably via injection, suitably through a narrow-bore needle) to the target site in a fluid or partially-fluid form. Suitably, the post-treatment composition of the invention is formed in situ within the target site (e.g. following transformation of a corresponding treatment composition). Transformations of the treatment composition into the post-treatment composition are described herein. Such transformations may be underway within the treatment composition during deliver thereof. This is particularly the case where the method of treatment involves forming the treatment composition by pre-mixing two or more compositions which, when mixed together, form the treatment composition. In a particular embodiment, an activatable composition and an activator composition, each as defined herein, may be premixed (suitably within a delivery device, e.g. double-barreled syringe) to form the treatment composition, and the transformation process may begin within the treatment composition. For example, physical transformations, such as swelling of an active precursor component, may begin following mixing. Chemical transformations, such as polymerization or crosslinking of an active precursor polymer, may begin following mixing, possibly in addition to physical transformation (in fact such physical transformation may be a pre-requisite for the chemical transformation). Suitably, however, the treatment composition is delivered to the target site whilst it retains sufficient fluidity to be injected (e.g. through a narrow bore needle). Suitably the treatment composition is suitably fluid to enable diffusion thereof into cracks, crevices, and slits within the target site before curing/hardening is completed to thereby furnish the post-treatment composition within the target site (especially within the cracks thereof). In this manner, it is preferable that pre-mixing (e.g. of an activatable composition and activator composition) occurs during the injection process, suitably immediately before a relevant outlet (e.g. before entering a needle or cannular). In a particular embodiment, the treatment composition is produced within and delivered from a double-barreled syringe comprising a mixing chamber.

Suitably the treatment composition cures to afford the post-treatment composition within no more than 1 hour, suitably within no more than 30 mins, suitably within no more than 10 mins, suitably within no more than 5 mins, suitably within no more than 2 mins. Suitably the treatment composition transforms into the post-treatment composition via transformation chemistry described herein. Most suitably the transformation involves pH-triggered swelling of a chemically-crosslinkable active precursor polymer (e.g. crosslinkable microgel particles) prior to free-radical-initiator-triggered chemical crosslinking thereof (which relies on pre-swelling to bring relevant crosslinkable groups into close proximity) resulting in an active component (suitably characterized as a network of intermolecularly crosslinked microgel particles).

The person skilled in the art will readily appreciate the treatment compositions of the invention may further comprise one or more additional therapeutic components and that, in such a manner, the treatment composition and/or post-treatment composition may be considered a carrier (e.g. carrier gel) for said therapeutic components. However, candidate subjects are suitably selected based on likely benefits brought thereto even by treatment compositions without any such additional one or more therapeutic agents.

It will be appreciated that the compositions of the present invention may be used as a monotherapy or, alternatively as an adjunct, or in combination with other known therapies.

Compositions of the invention may be delivered as a single administration (e.g. a single injection). Alternatively, the compositions may be delivered in multiple administrations, suitably at predetermined intervals.

### Treatment Outcomes and Aims

The treatment compositions and post-treatment compositions of the present invention suitably provide benefits to candidate subjects, suitably particularly at the target site(s) thereof, suitably as a consequence of the active component. The active component, which may be any suitable gel as described herein, suitably either enhances the target site (whether structurally, functionally, or both) or retards or prevents (further) degradation of the target site (again whether structurally, functionally, or both).

Suitably treatments of the invention, which suitably involve administration of a therapeutically-effective amount of a treatment and/or post-treatment composition to a target site of a candidate subject, aim to achieve one or more of the following treatment outcomes:
i) Revitalising one or more partially-degraded target site(s);
ii) Revitalising cells or cellular function associated or within one or more partially-degraded target site(s);
iii) Revitalising the extracellular matrices (ECMs) at one or more partially-degraded target sites;
iv) Revitalising cells surrounding or in close proximity to crevices, cracks, or slits within a target site and filled with the treatment and/or the post treatment composition;
v) Improving cellular nutrient diffusion at one or more partially-degraded target site(s);
vi) Retarding or inhibiting degradation at one or more partially-degraded target site(s);
vii) Retarding or inhibiting degradation of cells or cellular function associated or within one or more partially-degraded target site(s);
viii) Retarding or inhibiting degradation of the extracellular matrices (ECMs) at one or more partially-degraded target sites;
ix) Retarding or inhibiting degradation of cells surrounding or in close proximity to crevices, cracks, or slits within a target site;
x) Retarding or inhibiting degradation of cellular nutrient diffusion at one or more partially-degraded target site(s);
xi) Retarding or inhibiting biochemical degradation at one or more partially-degraded target site(s);
xii) Retarding or inhibiting structural or mechanical degradation at one or more partially-degraded target site(s);
xiii) Retarding or inhibiting progression of DDD at one or more partially-degraded target site(s), or retarding or inhibiting progression of DDD to the next stage on the Pfirrmann scale;
xiv) Treating or alleviating pain at one or more partially-degraded target site(s);
xv) Treating or alleviating discogenic pain at one or more partially-degraded IVD(s);
xvi) Reducing the risk or likelihood of future pain at one or more partially-degraded target site(s);
xvii) Reducing the risk or likelihood of a future requirement for surgery at one or more partially-degraded target site(s);
xviii) Increasing or facilitating maintenance of hydration at one or more partially-degraded target site(s);
xix) Treating or reducing inflammation or inflammatory responses at one or more partially-degraded target site(s);
xx) Inhibiting production of one or more inflammatory cytokines at one or more partially-degraded target site(s).
xxi) Restoring a positive balance of cytokine mediated anabolic/catabolic reaction in partially degraded target sites

### EXAMPLES

The present invention is described in more details by reference to the following non-limiting yet-illustrative examples.

### Materials and Equipment

Methyl methacrylate (MMA, 99%), methacrylic acid (MAA, 99%), ethyleneglycol dimethacrylate (EGDMA, 98%), ammonium persulfate (APS, 98%), Sodium dodecyl sulfate (SDS, 98.5%), potassium phosphate dibasic (K2HPO4, 99%) and glycidyl methacrylate (GMA, 97%) were purchased from Sigma Aldrich and used as received. L-ascorbic acid (AS, 99%) was purchased from scientific lab supplies and used as received. Chloroform and Sodium hydroxide (NaOH, 98%) were purchased from Fisher Scientific and used as received.

Particular embodiments of treatment compositions and post-treatment compositions applicable in the context of the present invention, and pertinent components thereof (e.g. microgels), are described in patent publication number WO2011/101684 (University of Manchester), for instance Methods 1 and 1A (paragraphs [00184-185]), Methods 3 and 3A (paragraph [00188-189]), Example 3 (paragraphs [00269-279]), and Example 4 (paragraphs [00280-317]), and Example 5 (paragraphs [00318-338]). However, descriptions of alternative methods of preparation are set forth in the Examples that follow.

### Example A—Preparation of Basic Internally-Crosslinked Microgel (Singly-Crosslinked Microgel, SXM)

A poly (MMA-MAA-EGDM) microgel is synthesised according to a modification of a literature procedure,¹³ and is also similar to that described in Method 1A, paragraph [00185], of the aforementioned patent document WO2011/101684, although in this example the proportions of MMA/MAA/EGDM are respectively 66.8 wt %/32.8 wt %/0.4 wt %—this equates to an approximate mol. % ratio of 320:185:1, or 63.2:36.6:0.2. The SXM—poly(MMA-MAA-EGDM) microgel is manufactured using a seed-feed emulsion polymerisation method. This is conducted in an aqueous environment under nitrogen, using sodium dodecyl sulfate (SDS) as the supporting surfactant, and ammonium persulfate APS as the thermo initiator at 85° C. The reaction duration is ~8 hours with the particle size and polydispersity measured throughout the reaction to monitor the quality of the material. The reaction is halted in ice, and filtered through a 53 micron filter for quality purposes. The SXM is then subjected to dialysis with distilled water to remove the surfactant material. This results in ~2 litres of 10 w/w % SXM material.

### Example B—Vinyl-functionalisation of Basic Microgel

A vinyl-functionalised (GMA-functionalised) poly (MMA-MAA-EGDM) microgel is also synthesised according to a modification of a literature procedure to yield (poly (MMA-MAA-EGDM)-GMA,¹³ and is again similar to that described in Methods 3 and 3A, paragraphs and of the aforementioned patent document WO2011/101684.

Essentially, 10% w/w SXM material from Example A is reacted with GMA over 18 hours to yield the functionalized material. At the end of the reaction the SXM-GMA is cooled and again filtered with a 53 micron filter to ensure quality. This is then washed with chloroform three times to remove any unreacted organic material. The material is then dialyzed again. Rotary evaporation is then employed to adjust the water content to the correct level needed to form the subsequent DXM (of Example C).

As the material is heat labile and radiation sensitive, aseptic filtration is the most appropriate method to ensure the sterilization of the gel, the functionalized SXM solution and the buffer.

Titration of the material evaluates the MAA content allowing determination of the structure and later determination of degree of functionalization with GMA, as per paragraphs [00192] and [00193] of WO2011/101684. The mol. % GMA in these examples is between 2 mol. % and 8 mol. %, generally about 5 mol. %.

### Example C—Preparation of Externally-Crosslinked Microgel (Doubly-Crosslinked Microgel, DXM)

An externally-crosslinked microgel can be produced via free-radical crosslinking of the aforementioned vinyl-functionalised (GMA-functionalised) poly(MMA-MAA-EGDM) microgel particles using a modification of a literature procedure to yield (poly(MMA-MAA-EGDM)-GMA,¹³ which is similar to that described in Example 3 (starting at paragraphs [00269]), Example 4 (starting at paragraph [00280]), and Example 5 (starting at paragraph [00318], of the aforementioned patent document WO2011/101684. However, in the context of the present invention, the relevant precursors are formulated for injection as follows.

An activatable composition (containing the key active precursor component, i.e. the vinyl-functionalised microgels obtained from Example B) and an activator formulation (containing ingredients to stimulate transformation of the active precursor component into an active component, i.e. to initiate free-radical inter-microgel crosslinking) were separately prepared in Vial A (Part A) and Vial B (Part B) respectively. In some experiments additional Part C was made up, as described below, and pre-mixed with Part A prior to use in a double-barreled syringe. The formulations are as follows:

| | | | | Part B | | Post-Mixed |
|---|---|---|---|---|---|---|
| | | | PART A values | values | | values |
| | | | (2.7 mL | (0.27 mL | | (2.97 mL |
| | Ingredient | Characteristics | altogether) | altogether) | Part C values | altogether) |
| PART A | Ascorbic | Mw 176.12 | 0.144 mL of | | | 2.536 mg |
| | acid (AA) | | 0.1M AA | | | 0.0144 mmol |
| | | | 0.1M AA = | | | 0.085 wt % |
| | | | 17.6 mg/mL | | | of Mixture |
| | | | 2.536 mg | | | |
| | | | 0.0144 mmol | | | |
| | | | 0.094 wt % of | | | |
| | | | Part A | | | |
| | GM-SXM | Avg particle size 60-90 nm | 2.556 mL of | | | 417 mg |
| | | wt % ratio | 16.3 wt % | | | 14 wt % of |
| | | (MMA/MAA/EGDM): | colloidal | | | mixture |
| | | 66.8:32.8:0.4, which equates | suspension | | | |
| | | to an approximate mol. % | 417 mg | | | |
| | | ratio 320:185:1 | 15.4 wt % of | | | |
| | | 2-8 mol % functionalisation | Part A | | | |
| | | with GMA | | | | |
| PART B | Ammonium | Mw 228.18 | | 3 wt % of | | 8.1 mg |
| | persulfate | | | 0.27 mL | | 0.035 mmol |
| | | | | 8.1 mg | | 0.27 wt % of |
| | | | | 0.035 | | mixture |
| | | | | mmol | | |
| | | | | 3 wt % of | | |
| | | | | Part B | | |
| | Sodium | Mw 40 | | 3.5 M in | | 37.8 mg |
| | hydroxide | | | 0.27 mL | | 0.945 mmol |
| | | | | 140 | | 1.27 wt % of |
| | | | | mg/mL | | mixture |
| | | | | 37.8 mg | | |
| | | | | 0.945 | | |
| | | | | mmol | | |
| | | | | 14 wt % of | | |
| | | | | Part B | | |
| PART C | Barium | Mw 233.38 | 0.18 g BaSO₄ | | 0.18 g | 0.18 g |
| | sulfate (with | | mixed with | | BaSO₄ | 771 mmol |
| | emulsification | | Part A (ignore | | (though 6 | 5.7 wt % of |
| | agents) | | 6 wt % | | wt % is | mixture |
| | | | emulsifiers). | | emulsifying | (assumes |
| | | | 6.25 wt % of | | ingredient)- | total of 3.15 |
| | | | Part A | | assume | mL/3.15 g |
| | | | (assumes total | | 100% | from 2.7 mL+ |
| | | | mass is 2.88 g | | BaSO₄ for | 0.27 mL + |
| | | | from 2.7 mL + | | calculations | 0.18 g) |
| | | | 0.18 g) | | 771 mmol | |
| | | | 771 mmol | | | |
| General | pH | | pH 5.6 | pH ~13 | Solid | pH 7.4 |
| Parameters | Solution/ | | Colloidal | Solution | | DXM |
| | Suspension | | suspension | | | Hydrogel |

### Vial A (Activatable Formulation): 2.7 mL (Aseptic)

0.144 mL 0.1M ascorbic acid
2.556 mL poly(MMA-MAA-EGD)-GM(aq) [16.3 wt % polymer] also known as SXM-GMA (the functionalised microgels obtained from Example B)

As such, Vial A was a 2.7 mL colloidal suspension containing 0.094 wt % ascorbic acid and 15.4 wt % GMA-functionalised microgel, with a pH of pH 5.6, such that the microgel particles adopted a collapsed configuration with an average particle size between 60 and 90 nm. The internal composition of the microgel particles is MMA/MAA/EGDMA with each monomer in a respective weight ratio (wt %) of 66.8:32.8:0.4 (approximately mol. % ratio of 320:185:1). The MMA/MAA/EGDMA microgel particles were 2-8 mol. % (typically about 5 mol. %). Sometimes the ingredients of Part C above are mixed with the contents of Vial A before being loaded into a syringe barrel. In some examples, this Part C is 0.18 g emulsifiable BaSO₄ which is mainly (94 wt %) BaSO₄ but with the following additional ingredients to facilitate emulsification when mixed with Part A:
2.1% w/w D-sorbitol
0.7% w/w sodium citrate
1.5% w/w simethicone
1.5% w/w PEG400
   0.18 g of the emulsifiable BaSO₄ is then premixed with the contents of vial A, resulting in a final Part A mixture containing approximately 6.25 wt % BaSO₄. The BaSO₄ system may, however, be omitted, and is only present to facilitate X-ray visualisation.

Vial B (Buffered Activator Formulation): 0.27 mL (Aseptic)
3 wt % ammonium persulfate (Aseptic)
3.5 M sodium hydroxide

As such, Vial B was a 0.27 mL solution containing the aforesaid ingredients, with a pH of ~pH 13. Herein, such a mixture may be considered a buffer, especially as when mixed Vial A the pH is reduced to around pH 7.4.

The contents of Vials A and B are loaded into a polycarbonate 10:1 volumetric ratio dual barrel syringe system via a filling adapter/vial-transfer device that is removably attachable to the syringe, such that one barrel contains the contents Vial A, and the other (smaller) barrel contains the contents of Vial B.

The filling adapter is then replaced with a mixing chamber to which a 70 mm 18G angiography needle is attached. The syringe is then ready for administering a treatment composition to a patient, which treatment composition is formed within the mixing chamber where the Vial A and Vial B compositions mix together prior to dispensation from the needle.

In an embodiment, the treatment composition may be delivered from the syringe via intradiscal injection procedures in skeletally mature patients with degenerative disc disease (DDD) at one level disc from L2 to S1. The total volume present in the syringe is approximately 3 mL (derived from Vials A and B), though generally only between 0.5 and 1 mL is actually delivered to an intervertebral disc. When the contents of the two barrels mix together, the resulting material has a pH of approximately pH 7.4.

The administration procedure involves injection of the treatment composition (Example C) from the double barrel syringe into the intervertebral disc. An image guided (C-arm) needle approaches the disc on the posterolateral aspect of the annulus, following a trajectory very similar to the well-established discography. The treatment composition would be injected by Healthcare professionals, trained on discography and image guided procedures (such as interventional radiologists, neurologists or rheumatologists). The injected treatment composition goes through a catalytic reaction and ultimately forms a stable polymer (post-treatment composition) in the nucleus of the disc which fills any tears in the disc and forms a robust physical structure which maintains the intervertebral disc space which should stop further deterioration of the structure. Using Example B in Part A of the treatment composition yields a post-treatment composition of doubly-crosslinked microgels (DXM), whereby microgel particles are themselves interlinked via covalent bonds formed through direct free-radical reactions between the alkene groups present within the surface-grafted GMA groups.

### Example 1—First Animal Model Study

To replicate the intended use in human patients, an animal model was used for injection of the DXM. The DXM material, which forms in situ following injection of a treatment composition of the invention (as per Example C above) has successfully undergone biocompatibility and biomechanical tests according to ISO 10993, ASTM F2346-05 (11) and ASTM F2789-10. To proceed from these tests an animal study was conducted.

Various animal and ‘degenerative’ models have been suggested for studies on lumbar discs.^{14,15,16,17} However these models involve inducing ‘artificial’ degenerative disc tissues thus may not be fully representative. As the intended use of the post-treatment compositions of the invention (e.g. doubly-crosslinked microgels, DXM) is to repair aged degenerate disc (DD) we used a model of naturally occurring age-related degeneration in the sheep. For this purpose, 10-12 years old sheep were selected.

A pilot study thereby ensured, using 3 animals aged 10-12 years old, to validate the suitability of a sheep model as a naturally degenerated intervertebral disc. To this end, the following procedures were followed.

### Procedure

3 sheep aged 10-12 years old were selected for the pilot animal model studies. All animals were raised by a certified farmer experienced in research on animals.

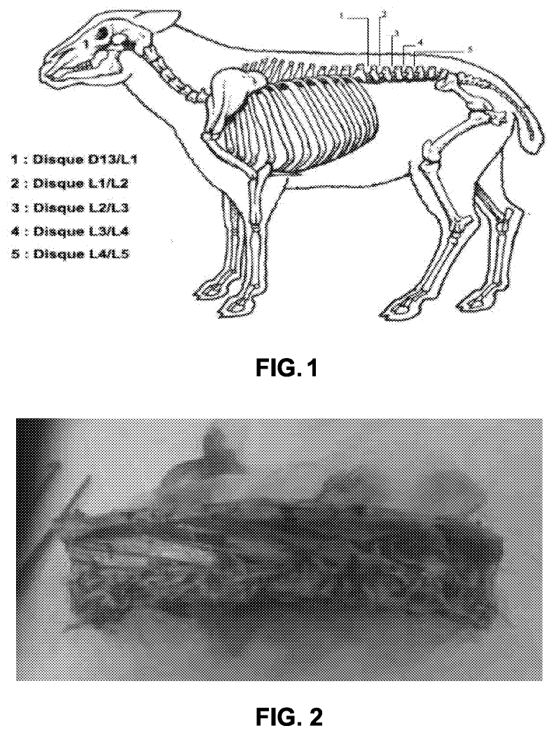

5 specific discs, all in the lumbar spinal region, were treated for each of the 3 sheep. FIG. 1 shows a sheep, and labels the specific vertebrae that were investigated/treated.
D13/L1 was the control disc and was not treated.
L1-L2 received a needle puncture but no treatment compositions.
L2-L3 received a physiological saline injection (phosphate-buffered saline, PBS) instead of a treatment composition.
L3-L4 received a treatment composition (Example C).
L4-L5 received a treatment composition (Example C).

A trained Interventional radiologist from the University Hospital of Bordeaux conducted the injection, of the treatment compositions as described in Example C (via double-barreled syringe), into the selected discs using the discography approach under X Ray navigation. The animals were anaesthetized and the area around the spine prepared for injection. Under radioscopy the discs were located and identified. An angiography needle was used for the 4 puncture/injection discs (CORDIS 502:652 70 mm 18G). Needle was introduced percutaneously through the psoas muscle as per standard discography procedure, visualised radiographically. 4:1 ratio (5 ml) double syringes were prepared using dual system of filter sterilised microgel and buffer solution containing initiators that induced polymerisation upon mixing. A 16:2, 4:1 mixing chamber and 18 gauge needle was used to deliver the DXM into the NP.

One month after the interventions the animals were sacrificed, and their lumbar spines harvested, placed in the tissue fixative of 10% neutral buffered formalin and sent to the molecular pathology laboratories of the IVD group in Manchester, a recognised world leader in the understanding of the biology of IVD disease and discogenic back pain, and their management.

FIG. 2 shows a photograph of an excised sheep lumbar spine.

The bone attached to the disc (Bony End Plate) was pared away to leave enough to support the disc, but insufficient to affect disc function, bone immediately next to the disc (the vertebral bony end plate).

These pieces of tissue were turned into appropriate sagittal “blocks” of IVD tissue (and associated bone). These are the supported and selected tissue from which histological sections are made.

Due to concerns about the integrity of the gel under the acid conditions usually used to decalcify bony tissue for histological examination for the pilot study an old, but still appropriate technique was used to cut the sections “undecalcified”. This required that Selotape is applied to the surface of the block and then sections are taken from below the Selotape which are then lifted from the surface of the block intact, attached to the Selotape.

Tissue sections were then stained with Haematoxylin and Eosin stains; the conventional stain used for morphological examination of all tissues.

Tissue sections were then subjectively assessed, quantitative measurements were obtained for important parameters. These included:
Disc height (distance between endplates)
Number of cells per sq millimetre
% of viable cells adjacent to the IVD
Presence of slit like and cyst like spaces
Loss of heamatox
Genera grade of degeneration

### Results

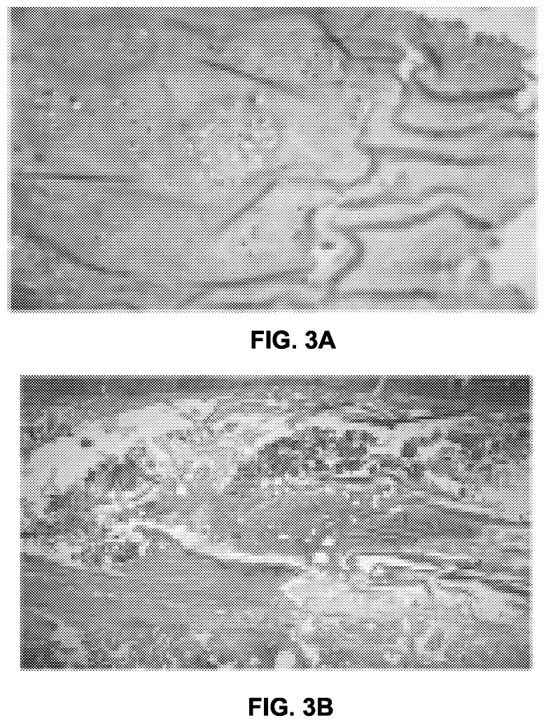

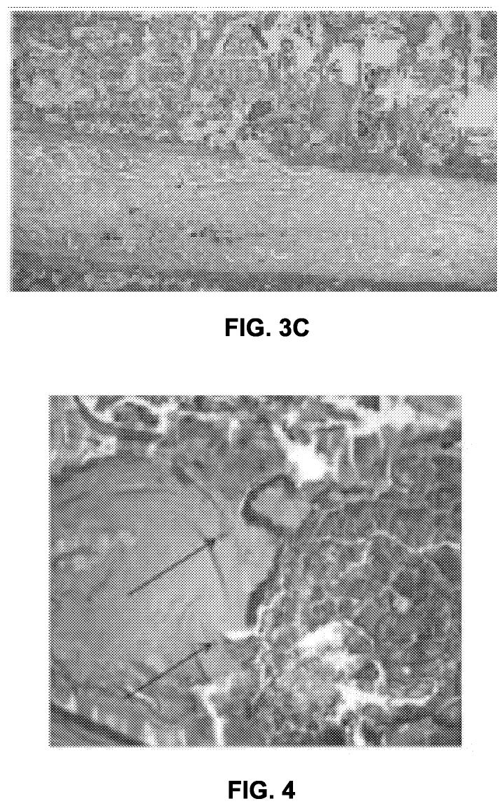

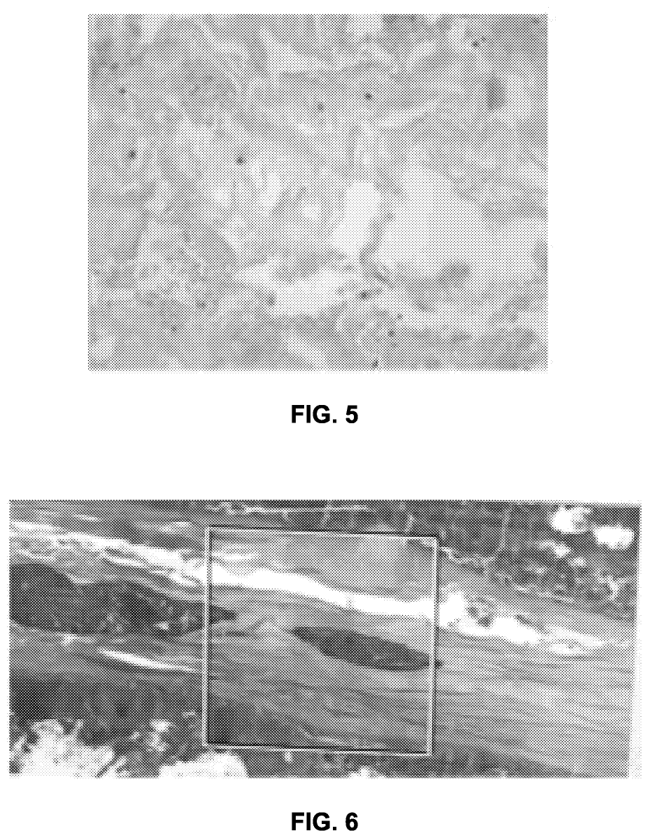

First tissues were assessed for evidence of spontaneous degeneration before assessing the discs that had received different interventions. Briefly the findings were:
The elderly animals used in this study have IVD with morphological evidence of degeneration (FIG. 3) similar to that seen in humans.
It is not clear whether the IVD in the lumbar spine vary in size naturally as it does in human spine but the IVD from nearer the cranial end of the lumbar spine were narrower than those from the caudal end.
It was not always possible to see the gel within the IVD because it tended to be physically removed during sectioning, but where it remained it was clearly seen (FIG. 4). Elsewhere spaces were left where it had been removed from the fixed (and therefore morphologically intact) tissue (FIG. 5).
When the gel was injected, it filled natural tears available in the degenerated IVD material (FIG. 6).
Even though there are differences in total cell numbers between all discs, there was no evidence of the gel reducing the number of cells or cell viability.

FIGS. 3A-C shows microscopic images of an IVD displaying evidence of degeneration by way of: A) cell clusters; B) slits; and C) end-plate lesions.

FIG. 4 show microscopic images of histological disc tissue (left) incorporating viable cells (tiny dark nuclei) and fragmented post-treatment composition (i.e. after curing in vivo) on the right.

FIG. 5 shows microscopic images of viable cells (dark round nuclei) next to spaces left by the post-treatment composition.

FIG. 6 shows a microscopic image of IVD disc tissue with DXM gel filling two separate tears within the disc.

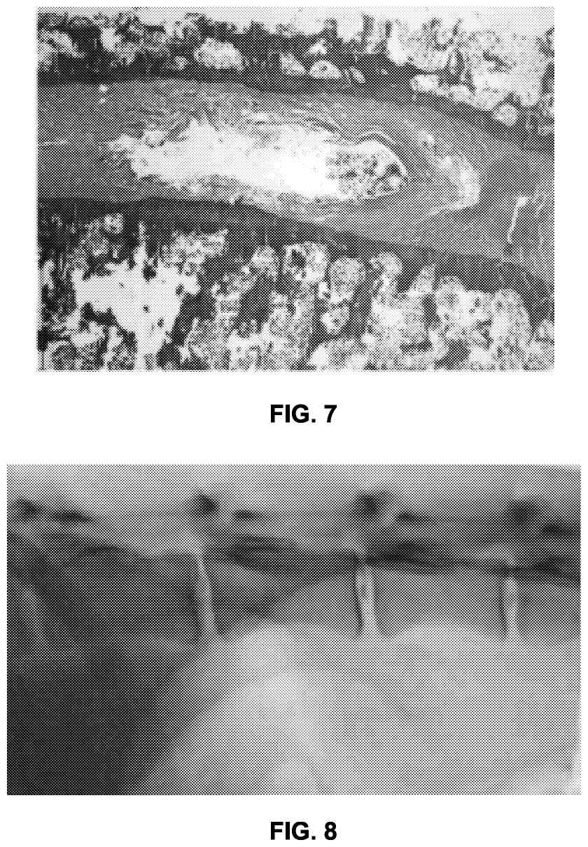

The discs that had received “sham” procedures were the lowest of all the IVD, where macro histology shows an empty disc and torn tissues. (FIG. 7)

FIG. 7 shows an microscopic image of IVD disc tissue injected with PBS, leaving mangled tissue in the centre of the disc.

Whether due to the gel or not, those discs in which gel had been injected all showed uniformly, a small increase in disc height over the “degenerate” control (i.e. untreated disc), which was the most caudal of all the discs examined. (see Table 1)

TABLE 1
Disc Height Measurements

| | | | | Number of | % of viable |
|---|---|---|---|---|---|
| DISC | | | Height | cells/sq mm | cells |
| 0011 | L1/2 | Puncture | 1.7 | 45 | 94 |
| | L1/2 | Puncture | 1.8 | 41 | 100 |
| | L2/3 | Serum | 1.9 | 56 | 63 |
| | L2/3 | Serum | 1.8 | 42 | 87 |
| | L3/4 | Gel | 2.2 | 47 | 100 |
| | L3/4 | Gel | 2.3 | 48 | 85 |
| | L4/5 | Gel | 2.4 | 39 | 84 |
| | L4/5 | Gel | 2.4 | 48 | 95 |
| | L5/6 | Control | 2.3 | 38 | 90 |
| | L5/6 | Control | 2.3 | 34 | 100 |
| | | | | Number | % of viable |
| DISC | | | Height | of cells | cells |
| 00004 | L1/2 | Puncture | 1.2 | 71 | 68 |
| yellow | L1/2 | Puncture | 1.1 | 60 | 84 |
| | L2/3 | Serum | 1.7 | 49 | 95 |
| | L2/3 | Serum | 1.7 | 50 | 94 |
| | L3/4 | Gel | 1.9 | 53 | 96 |
| | L3/4 | Gel | 1.9 | 59 | 95 |
| | L4/5 | Gel | 2 | 51 | 95 |
| | L4/5 | Gel | 2.1 | 46 | 95 |
| | L5/6 | Control | 2 | 52 | 89 |
| | L5/6 | Control | 2 | 54 | 96 |

NB. This table reports on discs that have been punctured (Puncture), discs that have been injected with serum (Serum), discs that have been injected with inventive compositions (Gel) and Controls.

The degree of “natural” degeneration, in which there is cell death and cell proliferation to form IVD cell clusters, made assessing changes in total and viable cell counts very difficult. There is no evidence that the gel reduces cell counts/unit area (FIG. 5), with cell counts within these discs in the same range as the non-gel discs (however, the non-gel discs have a very wide range of cell counts because of degeneration).

This preliminary study shows that aged sheep constitute an acceptable animal model for the study of degenerative intervertebral disc. They develop similar biomarkers to those that characterise degeneration of human discs.

The study reveals it is possible to inject a biocompatible, radio opaque biomaterial into the lumbar disc using the well-known and safe approach used to perform discography.

Degeneration of varying degree was seen in every disc of every animal. The characteristic feature of the degeneration was the presence of cyst-like and slit-like spaces which when injected with gel (which can be visualised in tissue sections) could be shown to be linked. The inventive gel permeated into cysts and along slits in every case.

There was an increase in the height of the discs treated by injection of DxM when compared to summated controls, sham operated and PBS injected discs even having taken into account the increasing thickness of the discs cranio-caudially, but this did not reach statistical significance (Mean: DxM 2.63+/−0.49, Untreated 2.42+/−0.47; p 0.08)

Numerous investigations have reported the low incidence of complication using the standard route used in a discography procedure.²⁰

The study confirms the injected material will interact as expected with the residual Nucleus Pulposus material to maintain the disc height, compared to control and shame disc.

The injected gel did not cause any adverse reaction within the disc as no inflammatory cells, or foreign body reaction has been noted histologically. The number of morphologically viable cells are in favour of an excellent tolerance of the material a month after injection.

The Nucleus Augmentation procedure is intrinsically safer than Nucleus replacement procedure as the gel is injected through a needle hole and hardens within the material of the IVD, preventing extrusion and migration as shown in the study.

The study also confirmed that insightful histology data could be obtained from excised discs.

### Example 2—Second Animal Model Study

Following the pilot study of Example 1, a pre-clinical study was performed with eight animals in order to evaluate the beneficial effects of the treatment compositions (of Example C) upon degenerated intervertebral disc. The treatment composition (as described in Example C) was administered to the vertebrae of 8 sheep via a standard discography procedure. The 8 sheep were differentiated by the following unique numerical identifiers:
10027
40026
10035
30044
60000
90011
50012
60085

For each of the 8 sheep, the same 5 vertebrae were treated in the same manner as depicted in Example 1. As such, five thoraco-lumbar and lumbar IVD from each animal were treated as follows (see FIG. 1 for an indication of relevant vertebrae):

$$D 13 / L 1 IVD = untreated control .$$

$$L 1 / 2 IVD = Sham procedure ( needle introduced into IVD but no injected material )$$

$$L 2 / 3 IVD = PBS ( phosphate buffered saline ) injection$$

$$L 4 / 5 and L 3 / 4 IVD = Double crosslinked gel ( DxM - Example C )$$

In these studies, the treatment compositions contained 6 wt % barium sulphate to allow for facile visualization by X-ray imaging (see Example C).

A trained Interventional radiologist from the University Hospital of Bordeaux conducted the injection, of the treatment compositions as described in Example C (via double-barreled syringe), into the selected discs using the discography approach under X Ray navigation. The animals were anaesthetized and the area around the spine prepared for injection. Under radioscopy the discs were located and identified. An angiography needle was used for the 4 puncture/injection discs (CORDIS 502:652 70 mm 18G). Needle was introduced percutaneously through the psoas muscle as per standard discography procedure, visualised radiographically. 4:1 ratio (5 ml) double syringes were prepared using dual system of filter sterilised microgel and buffer solution containing initiators that induced polymerisation upon mixing. A 16:2, 4:1 mixing chamber and 18 gauge needle was used to deliver the DXM into the NP.

FIG. 8 shows an X-ray image, captured via C-arm, showing the relevant vertebrae, with dark regions on the right hand two discs demonstrating the treatment gel within the disc.

The sheep were returned to the farm for 3 months. No adverse effects or ill health was observed during the 3 months, no sign of infection. After 3 months they were euthanised and five spine segments explanted into formalin. Explanted segments were sent to University of Manchester for further analysis-histology and immunohistochemistry.

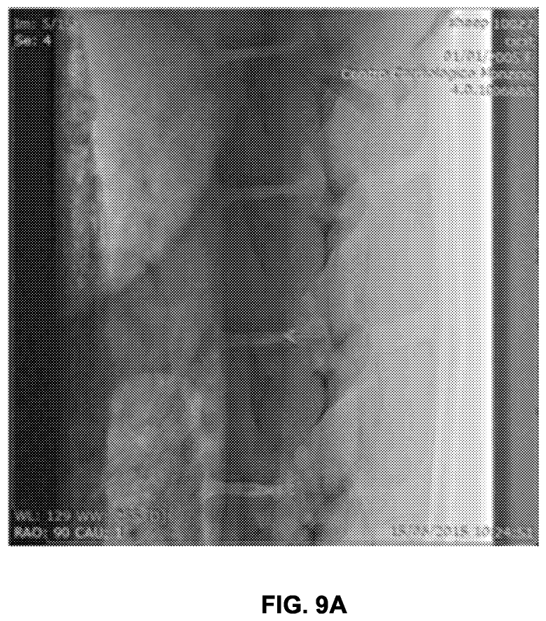

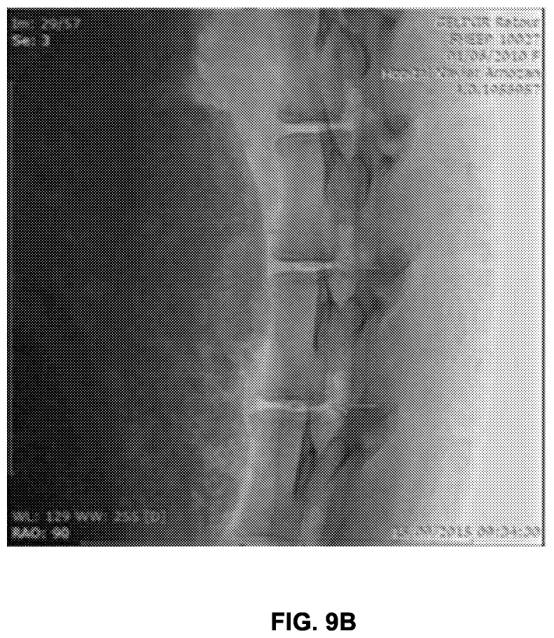

The administered inventive gel can be visualised at the time of injection and sacrifice as demonstrated in FIGS. 9A-B.

FIGS. 9A-B shows X-ray images: A) at the time of injection; and B) at the time of sacrifice.

The entire procedure may be described thus:
Animal is placed under radioscopy and the different discs are identified (last coast will serve as a benchmark),
An angiographic needle (Ref: Optimed 1201-1200, L: 70 mm, 18G) was introduced percutaneously, through the psoas muscle, according to the procedure described for a standard discography and the needle was pushed under visual control (radiography) to the center of the disc. D13-L1 control disc, L1-L2 needle puncture no injection, L2-L3 0.9% physiological saline injection approximately 1 mL, L3-L4 and L4-L5 disc received DXM injection of approximately 1 ml.
Radiographic controls were performed at regular intervals, supplied separately on CD
The punctures area was thoroughly cleaned and disinfected with betadine.
After 3 months follow-up, animals return to PTIB for sacrifice
15^{th} September 2015 for animals 90011, 10027, 40026 and 60085
2^{nd} October 2015 for animals 10035, 30044, 50012, and 60000
Each animal was checked over and weighed
The animals were premedicated with intramuscular ketamine at 10 mg/kg (Virbac solution 100 mg/mL) and Calmivet 5 mg (Acepromazine supplied by Vetroquinol 5 mg/ml)
They were euthanized by lethal injection of 30 cc Dolethal.
Discs D13-L1, L1-L2, L2-L3, L3-L4 and L4-L5 were excised into 10% neutral buffered formalin and then shipped to the University of Manchester UK for further testing.

### Tissue Processing

Following removal from the animal much of the vertebral bodies were removed from the IVD and the discs immersed in formalin.

Once they had reached the laboratory the IVD were bisected in the coronal plane. The two elements so produced were treated as follows:

### The Posterior Element

The posterior elements were decalcified in nitric acid, processed into paraffin wax using a conventional protocol and three sections made from each tissue block. These were stained with:
Haematoxylin and Eosin
Safranin-O (For proteoglycans)
Masson Trichrome.

These were used for assessing the nature, degree and distribution of degenerative changes, and the disc “height” (the distance between the two vertebra adjacent to a single disc.

### The Anterior Element

The anterior elements were decalcified in the chelating agent EDTA (this preserves immuno-detectable epitopes. They were then processed into paraffin wax and histological sections made.

The sections were used to investigate expression and distribution of three biomarkers of cell function using immunohistochemistry, a technique by the use of which target proteins can be identified and localised to tissues and cells in microscope sections:
Type II collagen. Although the function of type II collagen in the IVD is not fully understood, this particular biomarker is nevertheless one specific to the family of chondroid tissues of which the normal IVD centre (nucleus pulposus) is one. The molecule is synthesised by the cells of the nucleus pulposus and exported into the matrix. In degeneration, the phenotype of the cells changes with a failure to synthesise type II collagen and eventually redifferentiation into a cell that synthesises fibrous tissue collagens, notably types I and III.
Aggrecan. Aggrecan is a proteoglycan, synthesis of which is characteristic of cells of the chondroid lineage, which include the cells of the nucleus pulposus of the intervertebral disc. It is a proteoglycan that is highly hydrophilic pulling water molecules into its molecular super-structure with such avidity, the pressure developed can push apart adjacent vertebral bodies with sufficient force that in man they can do so even under the loads applying in a facultative biped.
Interleukin-1β. Interleukin-1 is a homeostatic regulator of cell function in the normal intervertebral disc. There are several isoforms. In IVD degeneration there is a relative overproduction of interleukin-1, particularly the isoform interleukin-1. This molecule is therefore a biomarker of “active degeneration”. The molecule, even when overproduced, is still present in small quantities.

The expression and distribution of gene product were described and semi-quantified using a conventional histological grading system of 0 to +++ (see Table 2 for details).

### Results and Discussion

Initial histology appeared positive, with no evidence of cell death or any adverse biocompatibility response. The Histology of the discs treated with the treatment compositions of Example C were comparable to control. These results reinforce those reported in the pilot animal study. The results also illustrate that for our pivotal clinical study aged sheep are a suitable model for intervertebral disc degeneration, and injection with the treatment compositions of the invention show no adverse effects after both 1 month and 3 month implantation.

### Degeneration

### Results

In every case the nature of the degeneration was such that in a human it would be described as early. It was characterised by the presence of cyst-like and slit-like spaces in the nucleus pulposus, formation of chondrones (cellular aggregates [the cells in the IVD are usually solitary] typical of degeneration, and subtle reduction in haematoxyphilia (a crude measure of loss of the highly negatively charged sulphate groups on Aggrecan).

The extent of degeneration varied from disc to disc and to a lesser degree between discs of different levels in the same animal.

The nature and extent of the degeneration is given in Table 1.

TABLE 2
Assessment of degeneration, disc height and gel penetration

| | | Grade of | Loss of | Cyst-like | Slit-like | Presence of | |
|---|---|---|---|---|---|---|---|
| | | degener′n | haematox | spaces | spaces | gel | |
| Animal | Disc | Graded 0 = none to +++ much or severe | | | | | Disc height |
| 10027 | D13/L1 | + | 0 | 0 | + | 0 | 1.7 |
| | L1/2 | +++ | ++ | +++ | ++ | 0 | 2.1 |
| | L2/3 | +++ | ++ | +++ | ++ | 0 | 2.1 |
| | L3/4 | +++ | + | ++ | ++ | Y | 2.2 |
| | L4/5 | ++ | + | ++ | ++ | Y | 2.3 |
| 40026 | D13/L1 | + | + | 0 | 0 | 0 | 2.0 |
| | L1/2 | + | + | 0 | + | 0 | 2.3 |
| | L2/3 | + | 0 | 0 | + | 0 | 2.4 |
| | L3/4 | + | 0 | + | + | Y | 2.4 |
| | L4/5 | ++ | + | ++ | + | Y | 2.6 |
| 10035 | D13/L1 | + | + | 0 | + | 0 | 2.2 |
| | L1/2 | 0 | 0 | 0 | 0 | 0 | 2.1 |
| | L2/3 | + | 0 | 0 | + | 0 | 2.1 |
| | L3/4 | + | + | 0 | + | 0 | 2.2 |
| | L4/5 | + | + | 0 | + | 0 | 2.3 |
| 30044 | D13/L1 | + | 0 | + | 0 | 0 | 2.4 |
| | L1/2 | +++ | + | ++ | 0 | 0 | 2.8 |
| | L2/3 | +++ | + | ++ | 0 | 0 | 3.2 |
| | L3/4 | +++ | + | ++ | 0 | Y | 3.0 |
| | L4/5 | +++ | + | ++ | 0 | Y | 3.1 |
| 60000 | D13/L1 | 0 | 0 | 0 | 0 | 0 | 2.4 |
| | L1/2 | + | + | 0 | + | 0 | 2.2 |
| | L2/3 | ++ | ++ | 0 | + | 0 | — |
| | L3/4 | ++ | + | + | ++ | Y | 2.2 |
| | L4/5 | + | + | 0 | + | Y | 2.7 |
| 90011 | D13/L1 | ++ | + | ++ | ++ | 0 | 2.1 |
| | L1/2 | + | + | ++ | 0 | 0 | 2.2 |
| | L2/3 | +++ | ++ | +++ | ++ | 0 | 2.4 |
| | L3/4 | +++ | ++ | +++ | + | Y | — |
| | L4/5 | +++ | ++ | +++ | ++ | Y | 2.3 |
| 50012 | D13/L1 | ++ | 0 | ++ | + | 0 | 3.0 |
| | L1/2 | ++ | 0 | ++ | + | 0 | 3.2 |
| | L2/3 | ++ | 0 | ++ | + | 0 | 3.8 |
| | L3/4 | ++ | 0 | ++ | + | Y | 3.7 |
| | L4/5 | ++ | 0 | ++ | + | Y | 3.4 |
| 60085 | D13/L1 | 0 | 0 | 0 | 0 | 0 | 2.0 |
| | L1/2 | + | 0 | + | 0 | 0 | 2.1 |
| | L2/3 | ++ | + | + | ++ | 0 | 2.4 |
| | L3/4 | ++ | + | + | ++ | Y | 2.1 |
| | L4/5 | +++ | +++ | ++ | ++ | Y | 2.2 |

NB: No obvious loss of demarcation was observed in the sheep between the annulus and nucleus pulposus

### Discussion

Degeneration of varying degree was seen in every disc of every animal. The characteristic feature of the degeneration was the presence of cyst-like and slit-like spaces which when injected with gel (which can be visualised in tissue sections) could be shown to be linked. Clusters were seen in 30044 L1/2, L3/4, L4/5.

Gel permeated into cysts and along slits in every case except 10035, where the degree of degeneration was low, and there was no cyst formation.

There was an increase in the height of the discs treated by injection of DxM when compared to summated controls, sham operated and PBS injected discs even having taken into account the increasing thickness of the discs cranio-caudially, but this did not reach statistical significance (Mean: DxM 2.63+/−0.49, Untreated 2.42+/−0.47; p 0.08)

### Biomarker Expression

In very general terms:
Type II collagen was expressed around cells in the nucleus pulposus and was also seen in adjacent IVD matrix. There was no detectable cytoplasmic expression. There was loss of collagen type II expression around cells adjacent to areas of cystic degeneration. This was not affected in any way by the nature of the intervention.
Aggrecan was expressed by cells of the nucleus pulposus and was also seen in the matrix particularly adjacent to the cells. This was not affected in any way by the nature of the intervention.
Interleukin-1β was not detected in the IVD

Specific data are given in Table 3.

TABLE 3
Biomarker expression

| | | Coll-II | | Aggrecan | | IL-1β | |
|---|---|---|---|---|---|---|---|
| | | | Areas of | | Areas of | | Areas of |
| | | Generally | degen | Generally | degen | Generally | degen |
| Animal | Disc | Graded 0 = none to +++ much | | | | | |
| 10027 | D13/L1 | C++ M++ | C0, M+ | C+++ M++ | C+++ M+ | 0 | 0 |
| | L1/2 | C++ M++ | C0, M+ | C+++ M++ | C+++ M+ | ++ | 0 |
| | L2/3 | C++ M++ | C0, M+ | C++ M++ | C+++ M+ | ++ | 0 |
| | L3/4 | C++ M++ | C0, M+ | C+++ M+ | C+++ M+ | ++ | 0 |
| | L4/5 | C++ M++ | C0, M+ | C+++ M++ | C+++ M+ | + | 0 |
| 40026 | D13/L1 | C++ M++ | C0, M+ | C+++ M++ | C+++ M+ | 0 | 0 |
| | L1/2 | C++ M++ | C0, M+ | C+++ M+ | C+++ M+ | 0 | 0 |
| | L2/3 | C++ M++ | C0, M+ | C+++ M++ | C+++ M+ | 0 | 0 |
| | L3/4 | C++ M++ | C0, M+ | C+++ M++ | C+++ M+ | 0 | 0 |
| | L4/5 | C++ M++ | C0, M+ | C+++ M++ | C+++ M+ | 0 | 0 |
| 10035 | D13/L1 | C++ M++ | C0, M+ | C+++ M++ | C+++ M+ | 0 | 0 |
| | L1/2 | C++ M++ | C0, M+ | C+++ M++ | C+++ M+ | 0 | 0 |
| | L2/3 | C++ M++ | C0, M+ | C++ M++ | C+++ M+ | 0 | 0 |
| | L3/4 | C++ M++ | C0, M+ | C+++ M++ | C+++ M+ | 0 | 0 |
| | L4/5 | C++ M++ | C0, M+ | C+++ M++ | C+++ M+ | 0 | 0 |
| 30044 | D13/L1 | C++ M++ | C0 M0 | C+++ M+ | C+++ M+ | 0 | 0 |
| | L1/2 | C0 M+ | C++ M+ | C+ M++ | C+++ M+ | C+ M0 | 0 |
| | L2/3 | C++ M+ | C0 M+ | C0 M+ | C++ M+ | 0 | 0 |
| | L3/4 | C0 M+ | C+ M+ | C++ M++ | C++ M++ | 0 | 0 |
| | L4/5 | C0 M+ | C+ M+ | C0 M+ | C++ M+ | 0 | 0 |
| 60000 | D13/L1 | C++ M++ | C0, M+ | C+++ M++ | C+++ M+ | 0 | 0 |
| | L1/2 | C++ M++ | C0, M+ | C+++ M+ | C+++ M+ | 0 | 0 |
| | L2/3 | C++ M++ | C0, M+ | C++ M++ | C+++ M+ | 0 | 0 |
| | L3/4 | C++ M++ | C0, M+ | C++ M++ | C+++ M+ | 0 | 0 |
| | L4/5 | C++ M++ | C0, M+ | C+++ M++ | C+++ M+ | 0 | 0 |
| 90011 | D13/L1 | C++ M+ | C0, M+ | C+++ M+ | C+++ M+ | 0 | 0 |
| | L1/2 | C++ M+ | C0, M+ | C+++ M++ | C+++ M+ | 0 | 0 |
| | L2/3 | C++ M+ | C0, M+ | C+++ M++ | C+++ M+ | C+ M0 | 0 |
| | L3/4 | C++ M+ | C0, M+ | C+++ M+ | C+++ M+ | 0 | 0 |
| | L4/5 | C++ M+ | C0, M+ | C+++ M++ | C+++ M+ | 0 | 0 |
| 50012 | D13/L1 | C++ M+ | C0, M+ | C+++ M++ | C+++ M+ | 0 | 0 |
| | L1/2 | C++ M+ | C0, M+ | C+++ M++ | C+++ M+ | 0 | 0 |
| | L2/3 | C++ M+ | C0, M+ | C+++ M++ | C+++ M+ | 0 | 0 |
| | L3/4 | C++ M+ | C0, M+ | C+++ M++ | C+++ M+ | 0 | 0 |
| | L4/5 | C++ M+ | C0, M+ | C+++ M++ | C+++ M+ | 0 | 0 |
| 60085 | D13/L1 | C++ M+ | C0 M+ | C+++ M+ | C+++ M+ | 0 | 0 |
| | L1/2 | C+++ M+ | C+ M+ | C++ M+ | C++ M+ | 0 | 0 |
| | L2/3 | C++ M+ | C0 M+ | C+++ M+ | C+++ M+ | 0 | 0 |
| | L3/4 | C+++ M+ | C0 M+ | C+++ M+ | C+++ M+ | 0 | 0 |
| | L4/5 | C+ M+ | C0 M+ | C+ M+ | C+++ M+ | 0 | 0 |

Aggrecan has a similar extracellular distribution to collagen II but is not lost in areas of degeneration. It is not affected by injection of DxM into the IVD.

Detectable IL-1β expression is very variable. It is only seen in IVD showing the most severe degeneration (+++). The number of discs expressing IL-1β is relatively low (6 of 40), but there is a distinct trend (that does not lend itself to statistical analysis because of sample size), showing that in severely degenerate discs (N=11) only 33% (2 of 6) of those injected with DxM express IL-1β, compared to 80% (4 of 5) that have not been injected with DxM. Were this to be sustained in a larger study it would mean that the molecular driver of degeneration is inhibited by DxM injection. Coupled with the trend towards greater height in discs injected with DxM this might be taken as early evidence that injection of DxM into severely degenerate IVD increases disc height and decreases the driver of degeneration, possibly by altering the mechanics of the IVD.

### SUMMARY

Injection of DxM into degenerate IVD of sheep that spontaneously develop degeneration:
Leads to spaces in the IVD becoming permeated by gel
Does not affect cell matrix synthesis even by cells immediately adjacent to the gel
Trends towards increasing disc height.
Trends towards decreasing expression of the molecule that is believed to drive the processes of degeneration.

### CONCLUSIONS

The anatomy of ovine disc has been shown to be a highly suitable model for the human spine, and is in fact the most frequently used species in intervertebral disc research. The discs have important and relevant similarities in geometrical and material properties as well as disc composition and annulus fibre orientation and the absence of notochordal cells. The gross anatomy between sheep and human discs is particularly similar in the lumbar region, with comparable values for collagen levels and type and water content in both the annulus and nucleus. It has also been reported in the literature (Smit 2002)²² that upon analysis of quadruped walking and standing, significant bending and torsion forces must be counterbalanced by tensile forces from muscles or ligaments resulting in axial compression being the main force factor on the spine, as seen in erect, upright humans. In addition it is also reported bone analyses have shown trabeculae run from end plate to end plate further confirming the main forces involved to be similar to those seen in erect humans, axial compression. In some cases these compressive forces have been found to be higher in sheep than those found in humans, hence making the sheep model a good and valid surrogate for human studies.

In conclusion, we believe that the ovine model is both an accepted and a very suitable model to demonstrate the efficacy of the methods of the invention in humans and animals. For similar reasons it is reasonable to extrapolate these results to the suitability of the inventive gels for treating Pfirrman Grade II/III partially degenerated IVDs in humans.

### CERTAIN ABBREVIATIONS

AF—Annulus Fibrosus
BDDA—1,4-butanediol diacrylate
CLBP—Chronic lower back pain
DDD—Degenerative Disc Disease
DXM—Double-crosslinked microgels (microgels externally linked together)
EA—ethylacrylate
ECM—Extra-cellular matrix
EGDM—ethyleneglycol dimethacrylate (same as EGDMA)
EGDMA—ethyleneglycol dimethacrylate
GMA—glycidyl methacrylate
IL-1—Interleukin 1
IL-1β—interleukin 1β
IVD—Intervertebral disc
MAA—methacrylic acid
MMA—methylmethacrylate
NP—Nucleus Pulposus
VEP—Vertebral Endplates

### REFERENCES

1. Erwin et al, J Can Chiropr Assoc 2014 September; 58 (3): 246-257.
2. Martin, B. I., et al. (2008) Expenditures and health status among adults with back and neck problems. JAMA 299 p. 656-664.
3. Antoniou J, Steffen T, Nelson F, Winterbottom N, Hollander A P, Poole R A, Aebi M, Alini M. (1996) The human lumbar intervertebral disc: evidence for changes in the biosynthesis and denaturation of the extracellular matrix with growth, maturation, ageing, and degeneration. J Clin Invest 98 (4) p. 996-1003.
4. Lotz J C, Ulrich J A. “Innervation, inflammation, and hypermobility may characterize pathologic disc degeneration: review of animal model data”, J Bone Joint Surg Am. 2006; 88 (Supl 2): 76-82.
5. Lotz J C et al, “New Treatments and Imaging Strategies in Degenerative Disease of the Intervertebral Disks, Radiology, Vol 264, Number 1 (July 2012), pages 6-19.
6. Anderson D G, Tannoury C. Molecular pathogenic factors in symptomatic disc degeneration. Spine J 2005; 5 (6 Suppl): 260S-266S.
7. Pfirrmann C W et al, Magnetic resonance classification of lumbar intervertebral disc degeneration. Spine (Phila Pa 1976) 2001; 26 (17): 1873-1878.
8. Shinmei M, Kikuchi T, Yamagishi M, Shimomura Y. The Role of interleukin-1 on proteoglycan metabolism of rabbit annulus fibrosus cells cultured in vitro. Spine (Phila Pa 1976) 1988; 13 (11): 1284-1290
9. Le Maitre C L, Freemont A J, Hoyland J A. The role of interleukin-1 in the pathogenesis of human intervertebral disc degeneration. Arthritis Res Ther 2005; 7 (4): R732-R745.
10. Le Maitre C L, Freemont A J, Hoyland J A. A preliminary in vitro study into the use of IL 1Ra gene therapy for the inhibition of intervertebral disc degeneration. Int J Exp Pathol 2006; 87 (1): 17-28.
11. Hedman, T. P., et al (1991) Design of an intervertebral disc prosthesis. Spine (16) p. S256-60.
12. Ray, C. D., (1991) Lumbar Interbody Threaded Prosthesis. In The Artificial Disc, Mayer, M. Brock Mayer; Weigel, HM; and Weigel, K, Editior, Springer-Verlag: Berlin, Heidelberg.
13. Liu, R.; Milani, A. H.; Freemont, T. J.; Saunders, B. R. Doubly crosslinked pH-responsive microgels prepared by particle inter-penetration: swelling and mechanical properties Soft Matter (2011), 7, pp. 4696-4704.
14. Theo Smit, The use of quadruped as an in vivo model for the study of the spine-biomechanical considerations, Eur Spine J (2002) 11: pp. 137-144
15. Mauro Alini et Al, Are animal models useful for studying human disc disorders/degeneration?Eur Spine J (2008) 17 pp. 2-19,
16. Turner A. S., Spine Surgery in a Large Animal Model: Experiences and Limitations. European Cells and Materials (2005) Vol. 10 S3, 34 ISSN pp. 1473-2262
17. J. E. Reid, J. R. Meakin, S. P. Robins, J. M. Skakle, D. W. Hukins Sheep lumbar intervertebral discs as models for human discs, Clinical Biomechanics (2002) 17 (4) pp. 312-314
18. Liu, R.; Milani, A. H.; Freemont, T. J.; Saunders, B. R. Doubly crosslinked pH-responsive microgels prepared by particle inter-penetration: swelling and mechanical properties Soft Matter (2011), 7, pp. 4696-4704.
19. http://www.medmix.ch/tl_files/pdf/L-System%20Brochure.pdf accessed Mar. 8, 2014
20. Stout, A., Discography Review Article Physical Medicine and Rehabilitation Clinics of North America, (2010) 21 (4) pp. 859-867
21. Fardon et al, “Lumbar disc nomenclature: version 2.0: Recommendations of the combined task forces of the North American Spine Society, the American Society of Spine Radiology and the American Society of Neuroradiology”, Spine J. 2014 Nov. 1; 14 (11): 2525-45.
22. Smit (2002). The use of a quadruped as an in vivo model for the study of the spine-biomechanical considerations. Eur Spine J 11 pp. 137-144
23. Reid et al (2002) Sheep lumbar intervertebral discs as models for human discs. Clin Biomech 17 pp. 312-314.

1. A method of treating a subject with early-stage degenerative disc disease (DDD) characterized by an intervertebral disc (IVD) exhibiting Grade II or III on the Pfirrmann scale, comprising:
i) providing an injection device comprising:
an activatable reservoir containing an activatable composition comprising an active precursor component;
an activator reservoir containing an activator composition comprising an activator agent, which promotes physical and/or chemical transformation of the active precursor component into an active component; and
a mixing chamber with an outlet;
wherein the activatable reservoir and the activator reservoir maintain separation between the activatable and activator compositions;
ii) operating the injection device to simultaneously:
deliver the activatable composition from the activatable reservoir and the activator composition from the activator reservoir into the mixing chamber to form a treatment composition; and
inject the treatment composition, via a needle or cannula, into a nucleus pulposus of the IVD;
wherein the combined volume of the activatable composition and activator composition used to form the treatment composition is between 0.1 and 3 mL;
mixing the activator composition with the activatable composition causes the active precursor component to swell and gel;
wherein the active precursor component physically and/or chemically transforms into the active component to provide, within the nucleus pulposus of the IVD, a non-biodegradable post-treatment composition comprising the active component;
wherein both the active precursor component and the active component exhibit pH-sensitive swelling and gelling properties;
wherein the post-treatment composition is relatively less fluidly mobile than the treatment composition;
wherein the treatment composition is fluidly mobile and the post-treatment composition is a gel.

2. The method as claimed in claim 1, wherein the injection device is or comprises a double-barreled syringe with a mixing chamber, wherein one barrel of the syringe contains the activatable composition and another barrel of the syringe contains the activator composition, and the syringe is operable to mix the activatable composition and activator composition within the mixing chamber.

3. The method as claimed in claim 1, wherein the volumetric ratio of the activatable composition to activator composition is between 4-15:1.

4. The method as claimed in claim 1, wherein the injection device comprises a needle, through which the treatment composition is dispensable, whose largest inner dimension (or inner diameter) is between 0.2 and 2 mm.

5. The method as claimed in claim 1, wherein:
the activatable composition comprises a particulate active precursor component that is pH-sensitive and susceptible to pH-dependent swelling and gelling such that particles of active precursor component increase in size in response to an increase in pH, and decrease in size in response to a decrease in pH;
the activator composition comprises a pH modifier.

6. The method as claimed in claim 5, wherein the activatable composition has a pH between 4 and 6.5, and the activator composition has a pH between 7 and 14.

7. The method as claimed in claim 1, wherein the activator composition further comprises a polymerisation initiator, and mixing the activator composition with the activatable composition further causes polymerisation of the active precursor component to form the active component.

8. The method as claimed in claim 1, wherein the early-stage degenerative disc disease (DDD) is characterized by an intervertebral disc (IVD) exhibiting Grade II on the Pfirrmann scale.

9. The method as claimed in claim 1, wherein upon injection the treatment composition diffuses into crevices, cracks, tears, or fissures present within the nucleus pulposus, and thereafter cures therein to form the post-treatment composition as a non-bolus hydrogel.

10. The method as claimed in claim 1, wherein the treatment composition comprises:
1-30 wt % of the active precursor component; and
sufficient quantities of the physical activator agent to furnish a pH above pH 5.

11. The method as claimed in claim 10, wherein the physical activator agent comprises a base.

12. The method as claimed claim 10, wherein the treatment composition comprises:
1-30 wt % of the active precursor component; and
0.001-6 wt % of the chemical activator agent.

13. The method as claimed in claim 12, wherein the chemical activator agent comprises an initiator, an accelerator, or an initiator and an accelerator.

14. The method as claimed in claim 13, wherein the initiator comprises ammonium persulphate and the accelerator comprises ascorbic acid.

15. The method as claimed in claim 1, wherein the treatment composition comprises:
1-30 wt % of the active precursor component and sufficient quantities of the physical activator agent to furnish a pH above pH 5;
0.001-5 wt % of an initiator; and
0.0001-2 wt % of an accelerator.

16. The method as claimed in claim 1, wherein the treatment composition further comprises a contrast agent and/or visualisation agent.

17. The method as claimed in claim 1, wherein the active precursor component comprises microgel particles bearing vinyl-containing moieties grafted onto their respective surfaces, and the activator agent comprises a free-radical initiator which promotes, optionally in the presence of an additional accelerator, direct inter-microgel particle crosslinking via free-radical coupling of the vinyl-containing moieties grafted to the surfaces of adjacent microgel particles.

18. The method as claimed in claim 1, wherein the method revitalises cells or cellular function associated with the intervertebral disc (IVD).

19. The method as claimed in claim 1, wherein the method retards or inhibits degradation of the intervertebral disc (IVD).

20. The method as claimed in claim 1, wherein the method improves cellular nutrient diffusion at the intervertebral disc (IVD).

21. The method as claimed in claim 1, wherein the method increases hydration of the intervertebral disc (IVD).

22. The method as claimed in claim 1, wherein the method treats or reduces inflammation or inflammatory responses at the intervertebral disc (IVD).

23. The method as claimed in claim 1, wherein the method inhibits production of one or more inflammatory cytokines at the intervertebral disc (IVD).

24. A method of treating a partially-degraded articular joint of a subject, comprising:
i) providing an injection device comprising:
an activatable reservoir containing an activatable composition comprising an active precursor component;
an activator reservoir containing an activator composition comprising an activator agent, which promotes physical and/or chemical transformation of the active precursor component into an active component; and
a mixing chamber with an outlet;
wherein the activatable reservoir and the activator reservoir maintain separation between the activatable and activator compositions;
ii) operating the injection device to simultaneously:
deliver the activatable composition from the activatable reservoir and the activator composition from the activator reservoir into the mixing chamber to form a treatment composition; and
inject the treatment composition, via a needle or cannula, into the extracellular matrix of the articular joint;
wherein the combined volume of the activatable composition and activator composition used to form the treatment composition is between 0.1 and 3 mL;
mixing the activator composition with the activatable composition causes the active precursor component to swell and gel;
wherein the active precursor component physically and/or chemically transforms into the active component to provide, within the articular joint, a non-biodegradable post-treatment composition comprising the active component;
wherein both the active precursor component and the active component exhibit pH-sensitive swelling and gelling properties;
wherein the post-treatment composition is relatively less fluidly mobile than the treatment composition;
wherein the treatment composition is fluidly mobile and the post-treatment composition is a gel.
